(19) United States
(12) Patent Application Publication
(10) Pub. No.: US 2014/0273103 A1
(43) Pub. Date: Sep. 18, 2014

(54) POLYSIALIC ACID, BLOOD GROUP ANTIGENS AND GLYCOPROTEIN EXPRESSION IN PROKARYOTES

(71) Applicant: Glycobia, Inc., Ithaca, NY (US)

(72) Inventors: Judith H. Merritt, Ithaca, NY (US); Adam C. Fisher, Ithaca, NY (US); Brian S. Hamilton, Ithaca, NY (US); Matthew P. DeLisa, Ithaca, NY (US)

(73) Assignee: Glycobia, Inc., Ithaca, NY (US)

(21) Appl. No.: 14/214,702

(22) Filed: Mar. 15, 2014

Related U.S. Application Data
(60) Provisional application No. 61/801,948, filed on Mar. 15, 2013.

(51) Int. Cl. C12P 19/18

(52) U.S. Cl. CPC C12P 19/18
USPC 435/97

(57) ABSTRACT

The invention described herein generally relates to glycoengineering host cells for the production of glycoproteins for therapeutic use. Host cells are modified to express biosynthetic glycosylation pathways. Novel prokaryotic host cells are engineered to produce N-linked glycoproteins wherein the glycoproteins comprise polysialic acid or blood group antigens.

### STATEMENT OF FEDERALLY SPONSORED RESEARCH AND DEVELOPMENT

This invention was made with government support under grant numbers 1R43GM093483-01, 5R43AI091336-01 and 5R43AI091336-02 by the National Institutes of Health. The government has certain rights in this invention.

### SEQUENCE LISTING

The instant application contains a Sequence Listing which has been submitted electronically in ASCII format and is hereby incorporated by reference in its entirety. Said ASCII copy, created on Mar. 6, 2014, is named GLY-200 SL.txt and is 91,817 bytes in size.

### FIELD OF INVENTION

The disclosure herein generally relates to the field of glycobiology and protein engineering. More specifically, the embodiments described herein relate to oligosaccharide compositions and production of therapeutic glycoproteins in recombinant hosts.

### BACKGROUND

Protein and peptide drugs have had a huge clinical impact and constitute a $70 billion market. Unfortunately, the efficacy of protein drugs is often compromised by limitations arising from proteolytic degradation, uptake by cells of the reticuloendothelial system, renal removal, and immunocomplex formation. This can lead to elimination from the blood before effective concentrations are reached, and can result in unacceptably short therapeutic windows. The predominant factors that contribute to these pharmacokinetic limitations are stability and immunogenicity. Efforts have been made to address these problems, including changing the primary structure, conjugating glycans or polymers to the protein, or entrapping the protein in nanoparticles to improve residence time and reduce immunogenicity. The most popular approach to date has been conjugation to monomethoxy poly(ethyleneglycol) (mPEG) commonly referred to as PEGylation. PEGylation can endow protein and peptide drugs with longer circulatory half-lives and reduce immunogenicity. A number of PEGylated drugs are now used clinically (e.g., asparaginase, interferon α, tumor necrosis factor and granulocyte-colony stimulating factor). However, PEG is not biodegradable via normal detoxification mechanisms and the administration of PEGylated proteins has been found to elicit anti-PEG antibodies.

PEGylation is a well-accepted approach to enhance stability and reduce immunogenicity, whereby protein is conjugated to poly(ethyleneglycol) (PEG) [10]. Such PEGylation involves the covalent attachment of either linear or branched chains of PEG via a chemically reactive side-chain, such as a hydroxysuccinimidylester or an aldehyde group, for linking to either the α or ε amino groups on the protein [11]. PEGylation can endow protein and peptide drugs with longer circulatory half-lives and reduced immunogenicity, as PEG is water-soluble and increases the size of the protein and reduces proteolytic cleavage by occluding cleavage sites [10]. The value of PEGylation was demonstrated for several proteins, including: (i) asparaginase [12], an enzyme used in the treatment of leukemia, and (ii) adenosine deaminase [13], which participates in purine metabolism. PEGylation was also used to enhance the activity of immunological factors such as granulocyte colony-stimulating factor (G-CSF), granulocyte-macrophage colony stimulating factor (GM-CSF) [14], tumor necrosis factor (TNF), interferon α-2a (IFN α-2a) and IFN α-2b [10]. While PEGylation is a chemical modification that can enhance pharmacokinetic properties, it is not without drawbacks. First, the heterogeneity of PEGylation yields many different isoforms of varying biological activity. This is primarily a result of the polydisperse nature of the polymer. Second, concerns have been raised about introducing a synthetic polymer into the human body that does not appear to be completely biodegradable [15]. Third, the extended half-life of PEGylated proteins that is often observed can be accompanied by reduced biological activity related to the structural change in the molecules as a result of conjugation [11]. Fourth, the process of PEGylation is expensive and requires several in vitro chemical reactions and multiple purifications [16]. Thus, while PEGylation has been clinically proven as a method to increase circulatory half-lives and reduce immunogenicity, clearly it is not the optimal solution.

An emerging clinical alternative to PEGylation is polysialylation which involves attachment of a polymer of natural N-acetylneuraminic acid (polysialic acid or PSA) to the protein. PSA is highly hydrophilic with similar hydration properties to PEG, is inconspicuous to the innate and adaptive immune systems, and is naturally synthesized and displayed on human cells. PSA has recently been developed for clinical use with polysialylated versions of insulin and erythropoietin each displaying improved tolerance and pharmacokinetics. Unfortunately, as with PEGylation, the PSA conjugation process is technically challenging and expensive making the final product cost prohibitive to the healthcare consumer. PSA conjugation requires the separate production and purification of the target protein and PSA, as well as the in vitro reductive amination of the nonreducing end of PSA to allow chemical linkage to primary amine groups on the protein.

PSA conjugation has proven to be a very effective method to increase the active life of therapeutic proteins and prevent them from being recognized by the immune system. PSA conjugation has several performance advantages over PEGylation and is currently being tested in the clinic.

Molecules that are inconspicuous to the innate and adaptive immune systems are likely to survive for prolonged periods in circulation. Polysialic acid (PSA; polymers of N-acetylneuraminic (sialic) acid) is one such molecule and offers a natural alternative to PEG as a conjugate that can modify serum persistence of proteins. PSA is a human polymer found almost exclusively on neural cell adhesion molecule (NCAM) where it has an antiadhesive function in brain development [17]. When used for protein and therapeutic peptide drug delivery, conjugated PSA provides a protective microenvironment. This increases the active life of the therapeutic protein in circulation and prevents it from being recognized by the immune system. Unlike PEG, PSA is metabolized as a natural sugar molecule by tissue sialidases [18]. The highly hydrophilic nature of PSA results in similar hydration properties to PEG, giving it a high apparent molecular weight in the blood. This increases circulation time since no receptors with PSA specificity have been identified to date [19].

While PSA is naturally found in the human body, it is also synthesized as a capsule by bacteria such as Neisseria meningitidis and certain strains of E. coli [20]. These polysialylated bacteria use molecular mimicry to evade the defense systems of the human body. Bacterial PSA is completely non-immunogenic, even when coupled to proteins, and is chemically identical to PSA in the human body to the extent that PSA has been developed for clinical use. Reductive amination of the nonreducing end of oxidized PSA allows in vitro chemical conjugation via primary amine groups on proteins, and the therapeutic benefits of PSA conjugation have been demonstrated with asparaginase [21] and insulin [22] for the treatment of leukemia and diabetes, respectively. Recent clinical data from trials with polysialylated insulin and erythropoietin showed that these biopharmaceuticals were well tolerated with enhanced pharmacokinetics [23]. Recently, two exciting discoveries have increased enthusiasm for PSA conjugation. First, it was observed that chemically polysialylated antitumor Fab fragments resulted in a 5-fold increase in bioavailability with a corresponding 3-fold increase in tumor uptake compared to unmodified Fab [24]. Second, site-specific (rather than random) coupling of PSA to engineered C-terminal thiols lead to antibody fragments with full immunoreactivity, increased blood half-life, higher tumor uptake, and improved specificity ratios [23]. PSA conjugation may add significant therapeutic value and polysialylated antibody fragments may be a viable alternative to whole IgGs by improving serum half-life and ameliorating concerns associated with Fc-domains.

Unfortunately, even PSA conjugation is not without its drawbacks. While effective in a therapeutic context, the production process of PSA conjugation is intensive and comes with a significant capital and processing cost. Currently, production involves a laborious eight-step process including: (i) fermentation of E. coli K1 and (ii) purification of its capsular coating, (iii) fermentation of E. coli expressing therapeutic protein and (iv) purification of therapeutic protein, (v) chemical cleavage of PSA from membrane anchor, (vi) purification of PSA, (vii) chemical crosslinking PSA to primary amine groups on the therapeutic protein by reductive amination of the nonreducing end of oxidized PSA, and (viii) purification of PSA-conjugated protein. This eight-step process requires two fermentations, two in vitro chemical reactions, and four purifications. The process is further complicated by the fact that standard amine-directed chemical conjugation of PSA results in random attachment patterns of undesirable heterogeneity [23]. To address this problem, site-specific, thiol-directed chemical conjugation can be used. However, this requires the addition of multiple C-terminal thiols, which are problematic to express in E. coli fermentation and require a mammalian expression system [23].

Accordingly, what is needed, therefore, is a method and composition for recombinant production of therapeutic proteins linked to an oligosaccharide composition that is structurally homogeneous and human-like produced in a controlled, rapid and cost-effective manner.

### SUMMARY

The present invention provides methods and compositions for the recombinant production of human or human-like glycans including polysialic acid on proteins. The present invention also provides methods and compositions for the recombinant production of human glycans including the T-antigen, Sialyl T-antigen, and the human blood group O H-antigen. The methods further provide for the production of non-native carbohydrates containing human glycans in prokaryotic host cells and attaching them as N-linked glycans to proteins. Various host cells are engineered to express proteins required to produce the necessary sugar nucleotides and glycosyltransferase activites required to synthesize specified oligosaccharide structures.

In certain aspects, a method is provided for producing an oligosaccharide composition comprising: culturing a recombinant host cell to express one or more of the enzyme activites comprising: GalNAc transferase (EC 2.4.1.-); galactosyltransferase (EC 2.4.1.-); fucosyltransferase (EC 2.4.1.69); and sialyltransferase (EC 2.4.99.4, EC 2.4.99.-, EC 2.4.99.8).

In one embodiment, the invention provides a glycoprotein composition comprising an N-linked sialic acid residue on the glycoprotein. Preferably, the glycoprotein composition comprising the N-linked sialic acid residue comprises one of following glycoforms: (Sia α2,8)ₙ-Sia α2,8-Sia α2,3-Gal1β1,3-GalNAc α1,3-GalNAc α1,3-GlcNAc; (Sia α2,8)ₙ-Sia α2,8-Sia α2,3-Galβ1,3-GalNAc α1,3-GlcNAc; and (Sia α2,8)ₙ-Sia α2,8-Sia α2,3-Galβ1,3-(GalNAc α1,3)ₙ. Alternatively, enzyme activities that convert UDP-GlcNAc to CMP-NeuNAc are introduced and expressed in a select host system. For instance, Neu enzyme activities that convert UDP-GlcNAc to CMP-NeuNAc comprise NeuB (synthase), NeuC (epimerase), and NeuA (synthase). In addition, enzyme activities required to synthesize polysialic acid and/or an acetylated form including NeuE, NeuS (polysialyltransferase), NeuD (O-acetyltransferase), and KpsCS are expressed. In certain embodiments, PSA is produced using minimal genes neuES and kpsCS to produce [α(2→3)Neu5Ac]ₙ; [α(2→6)Neu5Ac]ₙ; [α(2→8)Neu5Ac]ₙ or [α(2→9)Neu5Ac]ₙ or a combination thereof. In yet further embodiments, the glycoprotein composition has a defined degree of polymerization from about 1 to about 500, preferably between 2 and 125 sialic acid residues.

In various other aspects of the invention, a combination of glycosyltransferase enzymes are expressed to produce, for example, H-antigen (Fuc α1,2-Galβ1,3-GalNAc α1,3-GlcNAc); T-antigen (Galβ1,3-GalNAc α1,3-GlcNAc; Galβ1,3-GalNAc α1,3-GalNAc α1,3) and Sialyl T-antigen (Sia α2,3-Galβ1,3-GalNAc α1,3-GlcNAc).

While various host cells can be engineered to produce oligosaccharides and glycoprotein compositions, a preferred expression system involves prokaryotic host cells. Prokaryotic host cells further comprise an oligosaccharyl transferase activity for transfer of glycans comprising sialic acid residues onto a protein of interest.

### BRIEF DESCRIPTION OF THE FIGURES

FIG. 1 depicts representative biosynthetic pathways for the recombinant production of various human antigens and polysialic acid (PSA).

FIG. 2 represents FACS analysis of the engineered humanT antigen on the cell surface of bacteria detected by RCA (left), SBA (center) and glycosylated hGH detected by a SDS-PAGE (right).

FIG. 3 represents a MS of a recombinantly expressed human T antigen.

FIG. 4 represents a MS of a recombinantly expressed human sialyl T antigen on glucagon.

FIG. 5 represents a MS of recombinantly expressed human siayl T antigen on glucagon improved by expression of NeuDBAC on glucagon plasmid.

FIG. 6 represents MS of recombinantly expressed human sialyl T antigen on glucagon after treatment with α2,3 neuraminidase confirming sialylation and linkage.

FIG. 7 represents a dot blot of a recombinant PSA expression on the cell surface of E. coli MC4100 ΔnanA (A); and the expected linkages of an exemplary glycan (B).

FIG. 8 represents a Western blot using the aPSA antibodyin the presence of pJLic3BS-07 and NeuNAc supplementation (top) and total protein detected by the presence of the hexasitidine tag with αHis antiserum (bottom).

FIG. 9 represents a dot blot the effect of neuD expression on cell surface PSA.

FIG. 10 represents a SDS-PAGE and Western blot of anti-PSA (top) and anti-HIS (bottom) of ex vivo polysialylation of MBP4XGT with cstII-siaD fusion plasmid.

FIG. 11 represents a MS of a recombinantly expressed fucosylated human H antigen glycan with buffer control (A) or treated with α1,2 fucosidase and MS of a recombinantly expressed fucosylated H antigen glycan with expression of GDP-fucose biosynthetic genes (B).

FIG. 12 represents a Western blot of TNFαFab expressed with a pJK-07 glycosylation plasmid.

FIG. 13 represents MS of recombinant fucosylated glucagon peptide with the human H antigen (left) and the glucagon peptide with the GDP-fucose biosynthetic genes (right).

FIG. 14 represents recombinantly expressed fucosylated glucagon peptide after α1,2 fucosidase digest.

### ABBREVIATIONS AND TERMS

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a cell” includes one or a plurality of such cells. The term “or” refers to a single element of stated alternative elements or a combination of two or more elements, unless the context clearly indicates otherwise.

All publications, patents and other references mentioned herein are hereby incorporated by reference in their entireties.

EC numbers are established by the Nomenclature Committee of the International Union of Biochemistry and Molecular Biology (NC-IUBMB) (available at http://www.chem.qmul.ac.uk/iubmb/enzyme/). The EC numbers referenced herein are derived from the KEGG Ligand database, maintained by the Kyoto Encyclopedia of Genes and Genomics, sponsored in part by the University of Tokyo. Unless otherwise indicated, the EC numbers are as provided in the database as of March 2013.

The accession numbers referenced herein are derived from the NCBI database (National Center for Biotechnology Information) maintained by the National Institute of Health, U.S.A. Unless otherwise indicated, the accession numbers are as provided in the database as of March 2013.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

The term “claim” in the provisional application is synonymous with embodiments or preferred embodiments.

The term “human-like” with respect to a glycoprotein refers to proteins having attached either N-acetylglucosamine (GlcNAc) residue or N-acetylgalactosamine (GalNAc) residue linked to the amide nitrogen of an asparagine residue (N-linked) in the protein, that is similar or even identical to those produced in humans.

“N-glycans” or “N-linked glycans” refer to N-linked saccharide structures. The N-glycans can be attached to proteins or synthetic glycoprotein intermediates, which can be manipulated further in vitro or in vivo. The predominant sugars found on glycoproteins are are glucose (Glu), galactose (Gal), mannose (Man), fucose (Fuc), N-acetylgalactosamine (GalNAc), N-acetylglucosamine (GlcNAc), and sialic acid (e.g., N-acetyl-neuraminic acid (Neu5Ac, NeuAc, NeuNA, Sia or NANA). Hexose (Hex) refers to mannose or galactose.

The term “blood group antigens”, “BGA” or “human antigen” are used interchangeably and comprise an oligosaccharide moiet(ies).

The term “polysialic acid”, or “PSA” refers to an oligosaccharide structure that comprises at least two NeuNAc residues.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” or “substantially pure” nucleic acid or polynucleotide (e.g., RNA, DNA, or a mixed polymer) or glycoprotein is one which is substantially separated from other cellular components that naturally accompany the native polynucleotide in its natural host cell, e.g., ribosomes, polymerases and genomic sequences with which it is naturally associated. The term embraces a nucleic acid, polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleic acid, polynucleotide or glycoprotein so described has itself been physically removed from its native environment. For instance, an endogenous nucleic acid sequence in the genome of an organism is deemed “isolated” if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous coding sequence is considered “isolated” if it contains an insertion, deletion, or a point mutation introduced artificially, e.g., by human intervention. An “isolated nucleic acid” also includes a nucleic acid integrated into a host cell chromosome at a heterologous site and a nucleic acid construct present as an episome. Moreover, an “isolated nucleic acid” can be substantially free of other cellular material or substantially free of culture medium when produced by recombinant techniques or substantially free of chemical precursors or other chemicals when chemically synthesized.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

### DETAILED DESCRIPTION OF THE EMBODIMENTS

In various aspects, the present invention provides glycoengineered host cells to recombinatly produce oligosaccharides such as BGA-conjugated or PSA-conjugated proteins in a single fermentation without the added step for in vitro chemical modification. Advantageously, glycoengineered host expression technology enables control of the location and stoichiometry of attached polysaccharides and eliminates the need for excess thiols and in vitro chemical reactions. Accordingly, in certain embodiments, the present invention provides a method for producing an oligosaccharide composition comprising: culturing a recombinant host cell to express one or more of the enzymes comprising:
(i) GalNAc transferase activity (EC 2.4.1.-);
(ii) galactosyltransferase enzyme activity (EC 2.4.1.-);
(iii) fucosyltransferase enzyme activity (EC 2.4.1.69); and
(iv) sialyltransferase enzyme activity (EC 2.4.99.4, EC 2.4.99.-, EC 2.4.99.8).

FIG. 1 provides an overview of exemplary biosynthetic mechanisms to produce either BGA-conjugated or PSA-conjugated proteins in prokaryotes. In preferred embodiments, recombinant oligosaccharide synthesis is initiated by the expression of an α1,3-N-acetylgalactosamine transferase activity (EC 2.4.1.-). Additional embodiments include expression of other galactosyltransferase activity such as wbiP and cgtA to initiate recombinant oligosaccharide synthesis. Alternatively, recombinant oligosaccharide synthesis can be initiated directly on the N-linked site of the protein by expressing UDP-N-acetylglucosamine 4-epimerase activity (Rush et al (2010) JBC 285(3) 1671-1680). Accordingly, the present invention provides methods for recombinant oligosaccharide synthesis on either a GlcNAc reside or a GalNAc residue, which can be N-linked onto a protein of interest.

Human T Antigen

In exemplary embodiments, the invention provides methods to recombinantly express the genetic machinery needed for the production of various BGAs. A preferred method to produce the human T antigen comprises the recombinant expression of a GalNAc transferase activity (EC 2.4.1.-) that catalyzes the transfer of a UDP-GalNAc residue onto an acceptor substrate β1,4GlcNAc (EC 2.4.1.-). The host cell further expresses a galactosyltransferase enzyme activity (EC 2.4.1.-), which caps the GalNAc acceptor oligosaccharide resulting in a human T antigen. FIG. 3 provide experimental support of a recombinantly produced glycoform that correlates w the structure: Galβ1,3-GalNAc α1,3-GlcNAc, the human T antigen.

Human SialylT Antigen

In another aspect of the invention, a method is provided to produce the human sialyl T antigen, which comprises the recombinant expression of a GalNAc transferase activity (EC 2.4.1.-), a galactosyltransferase enzyme activity (EC 2.4.1.-) and a 2,3 NeuNAc transferase activity (EC 2.4.99.4). FIG. 4 represents a MS of a recombinantly produced glycoform on glucagon peptide that correlates w the structure: Sia α2,3-Galβ1,3-GalNAc α1,3-GlcNAc;

In more preferred embodiments, an improved level of a glycoform is produced by expressing one or more of the enzyme activites selected from a neuD sialic acid biosynthesis protein, N-acetylneuraminate synthase (EC 2.5.1.56), N-acetylneuraminate cytidylyltransferase (EC 2.7.7.43) and UDP-N-acetylglucosamine 2-epimerase (EC 5.1.3.14) DBAC. FIG. 5 describes a recombinantly produced glycoform on glucagon peptide with improved level of the sialyl T glycoform on the glucagon peptide. Addition of sialic acid was confirmed with the treatment of the glycosylated glucagon peptide with α2,3 neuraminidase FIG. 6.

Polysialic Acid

In other exemplary embodiments, the present invention provides a method for producing an oligosaccharide composition comprising: culturing a recombinant host cell to express one or more of the enzymes comprising: GalNAc transferase activity (EC 2.4.1.-) that transfers a GalNAc residue onto an acceptor substrate; galactosyltransferase enzyme activity (EC 2.4.1.-); fucosyltransferase enzyme activity (EC 2.4.1.69); and sialyltransferase enzyme activity (EC 2.4.99.4, EC 2.4.99.-, EC 2.4.99.8), wherein the host cell produces a polysialic acid.

Evidence of PSA on the cell wall is shown in FIGS. 7 and 9. The expected structural linkages of the PSA glycoforms include:

(Sia α2,8)ₙ-Sia α2,8-Sia α2,3-Galβ1,3-GalNAcα1,3-GalNAcα1,3-GlcNAc;

(Sia α2,8)ₙ-Sia α2,8-Sia α2,3-Galβ1,3-GalNAcα1,3-GlcNAc; and

(Sia α2,8)ₙ-Sia α2,8-Sia α2,3-Galβ1,3-(GalNAc α1,3)ₙ.

In select embodiments, the invention provides methods to recombinantly express the genetic machinery needed for the PSA production. As described in Example 12, the genes representing the capsular biosynthetic loci harboring the kps and neu genes of E. coli K1 and K92 are cloned into plasmid pACYC 184 for transformation of a preferred strain of E. coli.

In other select embodiments, the N-linked oligosaccharide compositions comprise or consists of [α(2→3)Neu5Ac]ₙ; [α(2→6)Neu5Ac]ₙ; [α(2→8)Neu5Ac]ₙ; [α(2→9)Neu5Ac]ₙ or a combination thereof.

Also disclosed are genes for producing the desired PSA oligosaccharide compositions. In certain embodiments, Neu activity such as NeuDBACES and Kps activity such as KpsSCUDEF are expressed. In yet other embodiments, one or more genes encoding KpsMT is attenuated. The invention provides a method for producing an N-linked sialic acid on a glycoprotein comprising: culturing a host cell to produce CMP-Neu5Ac from UDP-GlcNAc; PSA from CMP-Neu5Ac; and expressing an OST activity; wherein the OST activity transfers the sialic acid onto an acceptor asparagine of the resulting glycoprotein.

Preferably the oligosaccharide structure is N-linked to a protein, comprises a terminal sialic acid residue and is more preferably a polysialic acid that is a polysaccharide comprising at least 2 sialic acid residues joined to one another through α2-8 or α2-9 linkages. A suitable polysialic acid has a weight average molecular weight in the range 2 to 100 kDa, preferably in the range 1 to 35 kDa. The most preferred polysialic acid has a molecular weight in the range of 10-20 kDa, typically about 14 kDa.

More preferably, the N-linked PSA glycoprotein comprises about 2-125 sialic acid residues. Polymerized PSA can be transferred onto the glycoprotein, N-linked, some comprising 10-80 sialic acid residues, others 20-60 sialic acid residues, or 40-50 sialic acid residues. The preferred N-linked PSA glycoprotein composition has a defined degree of polymerization.

In additional embodiments, the glycoprotein composition further comprises a second N-linked oligosaccharide structure for example eukaryotic, human or human-like glycans such as Neu5Ac₁₋₄Gal₁₋₄GlcNAc₁₋₅Man₃GlcNAc₂, Man₃₋₅GlcNAc₁₋₂, GlcNAc₁₋₂, bacterial glycans such as GalNAc-α1,4-GalNAc-α1,4-[Glcβ1,3]GalNAc-α1,4-GalNAc-α1,4-GalNAc-α1,3-Bac-β1,N-Asn (GalNAc₅GlcBac, where Bac is bacillosamine or 2,4-diacetamido-2,4,6-trideoxyglucose). A mixture of N-linked PSA and N-linked oligosaccharide composition is also contemplated.

Glycoengineered E. coli have been used to attach diverse lipid-linked O-antigen glycans to corresponding asparagines in acceptor proteins in vivo (Feldman M F et al, (2005) Engineering N-linked protein glycosylation with diverse 0 antigen lipopolysaccharide structures in Escherichia coli. Proc Natl Acad Sci USA. 2005 Feb. 22; 102(8):3016-21). Enabling control of the location and stoichiometry of attached polysaccharides such as PSA may be critically important as amine-directed chemical conjugation of PSA is random and results in an unacceptably heterogeneous product. Favorable conjugation has only recently been achieved by site-specific, chemical coupling of PSA to engineered C-terminal thiols.

The PSA-conjugated protein is expected to improved circulating half-life and provide stability. Because PSA is a natural part of the human body, the recombinant PSA composition, which is chemically and immunologically similar to human PSA and (unlike PEG) is expected to be degraded or metabolized by tissue neuraminidases or sialidases to sialic acid residues. The recombinant PSA compositions are also immunologically invisible as a biodegrable polymer.

Additional advantages of the recombinant biosynthesis are as follows. While PSA conjugation requires several intricate in vitro chemical reactions and multiple purifications, direct recombinant production of PSA via host cell expression obviates the need for in vitro chemical reactions. There is no need to isolate PSA from E. coli K1 capsules prior to in vitro chemical crosslinking Random attachment patterns and undesirable heterogeneity resulting from the standard amine-directed chemical conjugation of PSA is also obviated. While site-specific, thiol-directed chemical conjugation can be used, this requires the appendage of multiple C-terminal thiols and expression from a mammalian host. Capital cost and production are kept low for efficient production and processing using the glycoengineered hosts. Therefore, in one aspect of the invention, the methods and host cells serve as a glycoprotein expression system for producing N-linked glycoproteins with structurally homogeneous human-like glycans and overcomes many of the above limitations and challenges. The host cells address the clear clinical demand for PSA-conjugated protein therapeutics.

Human H Antigen

In further exemplary embodiments, the present invention provides a method for producing an oligosaccharide composition comprising: culturing a recombinant host cell to express one or more of the enzymes comprising: GalNAc transferase activity that catalyzes a GalNAc residue onto an acceptor substrate (EC 2.4.1.-); galactosyltransferase enzyme activity (EC 2.4.1.-); and fucosyltransferase enzyme activity (EC 2.4.1.69). GDP-fucose transfer was confirmed with the treatment of the glycans with α1,2-fucosidase FIG. 11A. The recombinantly produced glycoform that correlates with the structure: α1,2 Fuc-Galβ1,3-GalNAcα1,3-GlcNAc, the human H antigen is shown in FIG. 11B. The human H antigen was also transferred onto a glucagon peptide by culturing the recombinant host to express a GDP-fucose biosynthetic machinery (Example 16). GDP-fucose transfer on glucagon was confirmed with the treatment of the glycans with α1,2-fucosidase FIG. 14. In an exemplary embodiment, TNFαFab heavy chain comprises a human H antigen via recombinant expression.

Prokaryotic Expression System

In preferred aspects, the invention provides a glycoprotein production system that serves as an attractive solution for circumventing the significant hurdles associated with eukaryotic cell culture systems or in vitro chemical conjugation. The use of bacteria as a production vehicle is expected to yield structurally homogeneous glycoproteins while at the same time dramatically lowering the cost and time associated with protein drug development and manufacturing. Other key advantages include: (i) the massive volume of data surrounding the genetic manipulation of bacteria; (ii) the established track record of using bacteria for protein production—30% of protein therapeutics approved by the FDA since 2003 are produced in E. coli bacteria; and (iii) the existing infrastructure within numerous companies for bacterial production of protein drugs.

Previously, the ability to attach a foreign glycan to an acceptor protein in E. coli has been shown (Wacker et al 2002 N-linked glycosylation in Campylobacter jejuni and its functional transfer into E. coli. Science 2002 Nov. 29; 298(5599):1790-3). Also, the ability to attach foreign glycans to a recombinant protein in a site-directed, stoichiometric manner using our proprietary C-terminal GlycTag has been demonstrated (PCT/US2009/030110). Moreover, the ability to attach lipid-linked polysaccharides (e.g., poly-FucNAc) to acceptor proteins in E. coli have been described (Feldman 2005). Recently, Valderrama-Rincon, et. al. (Valderrama-Rincon, et. al. “An engineered eukaryotic protein glycosylation pathway in Escherichia coli,” Nat. Chem. Biol. AOP (2012)) disclosed a biosynthetic pathway for the biosynthesis and assembly of Man₃GlcNAc₂ on Und-PP in the cytoplasmic membrane of E. coli, however, to date, no studies have demonstrated the ability to recombinantly produce BGA or PSA-conjugated proteins directly from an expression platform in a simple fermentation and purification process.

Nucleic Acid Sequences

In select embodiments, the invention provides isolated nucleic acid molecules, variants thereof, expression optimized forms of the disclosed genes, and methods of improvement thereon.

In one embodiment is provided an isolated nucleic acid molecule having a nucleic acid sequence comprising or consisting of glycosyltransferase gene homologs, variants and derivatives of the wild-type coding sequences. The invention provides nucleic acid molecules comprising or consisting of sequences which are structurally and functionally optimized versions of the wild-type genes. In a preferred embodiment, nucleic acid molecules and homologs, variants and derivatives comprising or consisting of sequences optimized for substrate affinity, specificity and/or substrate catalytic conversion rate, improved thermostability, activity at a different pH and/or optimized codon usage for improved expression in a host cell are provided.

In a further embodiment is provided nucleic acid molecules and homologs, variants and derivatives comprising or consisting of sequences which are variants of the glycosyltransferase genes having at least 60% identity. In a further embodiment provided nucleic acid molecules and homologs, variants and derivatives comprising or consisting of sequences which are variants having at least 62%, 65%, 68%, 70%, 75%, 79%, 80%, 81%, 82%, 83%, 84%, 85%, 90%, 92%, 95%, 98%, 99%, 99.9% or even higher identity to the wild-type gene.

In another embodiment, the encoded polypeptides having at least 50%, preferably, at least 55%, 60%, 70%, 80%, 90% or 95%, more preferably, 98%, 99%, 99.9% or even higher identity to the wild-type gene.

Provided also are nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing can be performed at temperatures about 5° C. lower than the Tₘ for the specific DNA hybrid under a particular set of conditions.

The nucleic acid molecule includes DNA molecules (e.g., linear, circular, cDNA, chromosomal DNA, double stranded or single stranded) and RNA molecules (e.g., tRNA, rRNA, mRNA) and analogs of the DNA or RNA molecules of the described herein using nucleotide analogs. The isolated nucleic acid molecule of the invention includes a nucleic acid molecule free of naturally flanking sequences (i.e., sequences located at the 5′ and 3′ ends of the nucleic acid molecule) in the chromosomal DNA of the organism from which the nucleic acid is derived. In various embodiments, an isolated nucleic acid molecule can contain less than about 10 kb, 5 kb, 4 kb, 3 kb, 2 kb, 1 kb, 0.5 kb, 0.1 kb, 50 bp, 25 by or 10 by of naturally flanking nucleotide chromosomal DNA sequences of the microorganism from which the nucleic acid molecule is derived.

The genes, as described herein, include nucleic acid molecules, for example, a polypeptide or RNA-encoding nucleic acid molecule, separated from another gene or other genes by intergenic DNA (for example, an intervening or spacer DNA which naturally flanks the gene and/or separates genes in the chromosomal DNA of the organism).

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

In another embodiment, an isolated glycosyltransferase gene encoding nucleic acid molecule hybridizes to all or a portion of a nucleic acid molecule having the nucleotide sequence set forth in the sequence listings or hybridizes to all or a portion of a nucleic acid molecule having a nucleotide sequence that encodes a polypeptide having the amino acid sequence of any of amino acid sequences as set forth in the sequence listings. Such hybridization conditions are known to those skilled in the art (see, for example, Current Protocols in Molecular Biology, Ausubel et al., eds., John Wiley & Sons, Inc. (1995); Molecular Cloning: A Laboratory Manual, Sambrook et al., Cold Spring Harbor Press, Cold Spring Harbor, N.Y. (1989)). In another embodiment, an isolated nucleic acid molecule comprises a nucleotide sequence that is complementary to a neu or kps gene encoding nucleotide sequence as set forth herein.

The nucleic acid sequence fragments display utility in a variety of systems and methods. For example, the fragments may be used as probes in various hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hybridization to target sequences can be detected and optionally quantified. One of skill in the art will appreciate that the nucleic acid fragments may be used in a wide variety of blotting techniques not specifically described herein.

It should also be appreciated that the nucleic acid sequence fragments disclosed herein also find utility as probes when immobilized on microarrays. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of each of which is incorporated herein by reference in its entirety.

As is well known in the art, enzyme activities are measured in various ways. For example, the pyrophosphorolysis of OMP may be followed spectroscopically. Grubmeyer et al., J. Biol. Chem. 268:20299-20304 (1993). Alternatively, the activity of the enzyme is followed using chromatographic techniques, such as by high performance liquid chromatography. Chung and Sloan, J. Chromatogr. 371:71-81 (1986). As another alternative the activity is indirectly measured by determining the levels of product made from the enzyme activity. More modern techniques include using gas chromatography linked to mass spectrometry (Niessen, W. M. A. (2001). Current practice of gas chromatography—mass spectrometry. New York, N.Y.: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G., R. O. Dunn, and M. O. Bagby. 1997. Biodiesel: The use of vegetable oils and their derivatives as alternative diesel fuels. Am. Chem. Soc. Symp. Series 666: 172-208), physical property-based methods, wet chemical methods, etc. are used to analyze the levels and the identity of the product produced by the organisms. Other methods and techniques may also be suitable for the measurement of enzyme activity, as would be known by one of skill in the art.

Another embodiment comprises mutant or chimeric nucleic acid molecules or genes. Typically, a mutant nucleic acid molecule or mutant gene is comprised of a nucleotide sequence that has at least one alteration including, but not limited to, a simple substitution, insertion or deletion. The polypeptide of said mutant can exhibit an activity that differs from the polypeptide encoded by the wild-type nucleic acid molecule or gene. Typically, a chimeric mutant polypeptide includes an entire domain derived from another polypeptide that is genetically engineered to be collinear with a corresponding domain. Preferably, a mutant nucleic acid molecule or mutant gene encodes a polypeptide having improved activity such as substrate affinity, substrate specificity, improved thermostability, activity at a different pH, improved soluability, improved expression, or optimized codon usage for improved expression in a host cell.

Isolated Polypeptides

In one embodiment, polypeptides encoded by nucleic acid sequences are produced by recombinant DNA techniques and can be isolated from expression host cells by an appropriate purification scheme using standard polypeptide purification techniques. In another embodiment, polypeptides encoded by nucleic acid sequences are synthesized chemically using standard peptide synthesis techniques.

Included within the scope of the invention are glycosyltransferase polypeptides or gene products that are derived polypeptides or gene products encoded by naturally-occurring bacterial genes. Further, included within the inventive scope, are bacteria-derived polypeptides or gene products which differ from wild-type genes, including genes that have altered, inserted or deleted nucleic acids but which encode polypeptides substantially similar in structure and/or function.

For example, it is well understood that one of skill in the art can mutate (e.g., substitute) nucleic acids which, due to the degeneracy of the genetic code, encode for an identical amino acid as that encoded by the naturally-occurring gene. This may be desirable in order to improve the codon usage of a nucleic acid to be expressed in a particular organism. Moreover, it is well understood that one of skill in the art can mutate (e.g., substitute) nucleic acids which encode for conservative amino acid substitutions. It is further well understood that one of skill in the art can substitute, add or delete amino acids to a certain degree to improve upon or at least insubstantially affect the function and/or structure of a gene product (e.g., glycosyltransferase activity) as compared with a naturally-occurring gene product, each instance of which is intended to be included within the scope of the invention. For example, the glycosyltransferase ctivity, enzyme/substrate affinity, enzyme thermostability, and/or enzyme activity at various pHs can be unaffected or rationally altered and readily evaluated using the assays described herein.

In various aspects, isolated polypeptides (including muteins, allelic variants, fragments, derivatives, and analogs) encoded by the nucleic acid molecules are provided. Preferably the isolated polypeptide has preferably 50%, 60%-70%, 70%-80%, 80%-90%, 90%-95%, 95%-98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or even higher identity to the sequences optimized for substrate affinity and/or substrate catalytic conversion rate.

According to other embodiments, isolated polypeptides comprising a fragment of the above-described polypeptide sequences are provided. These fragments preferably include at least 20 contiguous amino acids, more preferably at least 25, 30, 35, 40, 45, 50, 60, 70, 80, 90, 100 or even more contiguous amino acids.

The polypeptides also include fusions between the above-described polypeptide sequences and heterologous polypeptides. The heterologous sequences can, for example, include sequences designed to facilitate purification, e.g. histidine tags, and/or visualization of recombinantly-expressed proteins. Other non-limiting examples of protein fusions include those that permit display of the encoded protein on the surface of a phage or a cell, alter the subcellular localization of the protein, fusions to intrinsically fluorescent proteins, such as green fluorescent protein (GFP), and fusions to the IgG Fc region.

Secretion Signal Sequences

In selected embodiments, the oligosaccharide-conjugated polypeptide is expressed with a secretion signal sequence. The secretion signal can be an amino terminal sequence that facilitates transit across a membrane. In those embodiments where the host organism is prokaryotic, secretion signal is a leader peptide domain of a protein that facilitates insertion into the membrane or transport through a membrane. The signal sequence is removed after crossing the inner membrane, and proteins may be retained in the periplasmic space.

Various secretion signals are used, for instance pelB. The predicted amino acid residue sequences of the secretion signal domain from two PelB gene product variants from Erwinia carotova are described in Lei et al., Nature, 331:543-546 (1988). The leader sequence of the PelB protein has previously been used as a secretion signal for fusion proteins (Better et al., Science, 240:1041-1043 (1988); Sastry et al., Proc. Natl. Acad. Sci., USA, 86:5728-5732 (1989); and Mullinax et al., Proc. Natl. Acad. Sci., USA, 87:8095-8099 (1990)). Amino acid residue sequences for other secretion signal polypeptide domains from E. coli useful in this invention include those described in Oliver, Escherichia coli and Salmonella Typhimurium, Neidhard, F. C. (ed.), American Society for Microbiology, Washington, D.C., 1: 56-69 (1987).

Another typical secretion signal sequence is the gene III (gill) secretion signal. Gene HI encodes Pill, one of the minor capsid proteins from the filamentous phage fd (similar to Ml 3 and rl). Pill is synthesized with an 18 amino acid, amino terminal signal sequence and requires the bacterial Sec system for insertion into the membrane.

Another typical secretion signal sequence is the SRP secretion signal. SRP secretion signals have been used, for example, to improve production of fusion protein for phage display (Steiner et al. Nat. Biotechnology, 24:823-831 (2006)). Most commonly used type II secretion signals, such as the PelB secretion signal, use the SecB pathway. Thus, secretion constructs presented herein for expression of human mAb heavy and light chains use an SRP secretion signal, namely the secretion signal of the E. coli dsbA gene. Other SRP secretion signals that can be used in the methods, polynucleotides and polypeptides provided herein include SfmC (chaperone), ToIB (translocation protein), and TorT (respiration regulator). The sequences of these signals are known in the art.

Secrection by the E. coli SecB mechanism involves attachment of a nascent polypeptide first to trigger factor, TF, and then to SecB. The ScB protein then directs attachment of the completed polypeptide to the Type II secretion complex which secretes the protein into the periplasm. Without being bound by theory, it is thought that some recombinant proteins may fold into forms which secrete poorly by this mechanism. In contrast, the SRP mechanism recognizes a different set of secretion signals and directs co-translation and secretion of nascent polypeptides through the Type II secretion complex into the periplasm. This mechanism can be used to avoid problems that could occur in secretion by the SecB pathway.

It will be apparent to one of ordinary skill in the art that any suitable secretion signal sequence may be used to facilitate secretion of expressed polypeptides.

Secretion of Proteins into Periplasm and Medium

To determine secretion of an active antibody into culture the medium, media samples collected during the expression analysis of the variousP constructs are assayed by ELISA for its antigen binding activity.

The polynucleotides or nucleic acid molecules of the present invention refer to the polymeric form of nucleotides of at least 10 bases in length. These include DNA molecules (e.g., linear, circular, cDNA, chromosomal, genomic, or synthetic, double stranded, single stranded, triple-stranded, quadruplexed, partially double-stranded, branched, hair-pinned, circular, or in a padlocked conformation) and RNA molecules (e.g., tRNA, rRNA, mRNA, genomic, or synthetic) and analogs of the DNA or RNA molecules of the described as well as analogs of DNA or RNA containing non-natural nucleotide analogs, non-native inter-nucleoside bonds, or both. The isolated nucleic acid molecule of the invention includes a nucleic acid molecule free of naturally flanking sequences (i.e., sequences located at the 5′ and 3′ ends of the nucleic acid molecule) in the chromosomal DNA of the organism from which the nucleic acid is derived. In various embodiments, an isolated nucleic acid molecule can contain less than about 10 kb, 5 kb, 4 kb, 3 kb, 2 kb, 1 kb, 0.5 kb, 0.1 kb, 50 bp, 25 by or 10 by of naturally flanking nucleotide chromosomal DNA sequences of the microorganism from which the nucleic acid molecule is derived.

The heterologous nucleic acid molecule is inserted into the expression system or vector in proper sense (5′→3′) orientation relative to the promoter and any other 5′ regulatory molecules, and correct reading frame. The preparation of the nucleic acid constructs can be carried out using standard cloning methods well known in the art, as described by Sambrook et al., Molecular Cloning: A Laboratory Manual, Cold Springs Laboratory Press, Cold Springs Harbor, N.Y. (1989), which is hereby incorporated by reference in its entirety. U.S. Pat. No. 4,237,224 to Cohen and Boyer, which is hereby incorporated by reference in its entirety, also describes the production of expression systems in the form of recombinant plasmids using restriction enzyme cleavage and ligation with DNA ligase.

Suitable expression vectors include those which contain replicon and control sequences that are derived from species compatible with the host cell. For example, if E. coli is used as a host cell, plasmids such as pUC19, pUC18, or pBR322 may be used. Other suitable expression vectors are described in Molecular Cloning: a Laboratory Manual: 3rd edition, Sambrook and Russell, 2001, Cold Spring Harbor Laboratory Press, which is hereby incorporated by reference in its entirety. Many known techniques and protocols for manipulation of nucleic acids, for example in preparation of nucleic acid constructs, mutagenesis, sequencing, introduction of DNA into cells and gene expression, and analysis of proteins, are described in detail in Current Protocols in Molecular Biology, Ausubel et al. eds., (1992), which is hereby incorporated by reference in its entirety.

Different genetic signals and processing events control many levels of gene expression (e.g., DNA transcription and messenger RNA (“mRNA”) translation) and subsequently the amount of fusion protein that is displayed on the ribosome surface. Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is often desirable to use strong promoters to obtain a high level of transcription and, hence, expression and surface display. Therefore, depending upon the host system utilized, any one of a number of suitable promoters may also be incorporated into the expression vector carrying the deoxyribonucleic acid molecule encoding the protein of interest coupled to a stall sequence. For instance, when using E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Translation of mRNA in prokaryotes depends upon the presence of the proper prokaryotic signals, which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Host Cells

In accordance with the present invention, the host cell may be a prokaryote. Such cells serve as a host for expression of recombinant proteins for production of recombinant therapeutic proteins of interest. Exemplary host cells include E. coli and other Enterobacteriaceae, Escherichia sp., Campylobacter sp., Wolinella sp., Desulfovibrio sp. Vibrio sp., Pseudomonas sp. Bacillus sp., Listeria sp., Staphylococcus sp., Streptococcus sp., Peptostreptococcus sp., Megasphaera sp., Pectinatus sp., Selenomonas sp., Zymophilus sp., Actinomyces sp., Arthrobacter sp., Frankia sp., Micromonospora sp., Nocardia sp., Propionibacterium sp., Streptomyces sp., Lactobacillus sp., Lactococcus sp., Leuconostoc sp., Pediococcus sp., Acetobacterium sp., Eubacterium sp., Heliobacterium sp., Heliospirillum sp., Sporomusa sp., Spiroplasma sp., Ureaplasma sp., Erysipelothrix, sp., Corynebacterium sp. Enterococcus sp., Clostridium sp., Mycoplasma sp., Mycobacterium sp., Actinobacteria sp., Salmonella sp., Shigella sp., Moraxella sp., Helicobacter sp, Stenotrophomonas sp., Micrococcus sp., Neisseria sp., Bdellovibrio sp., Hemophilus sp., Klebsiella sp., Proteus mirabilis, Enterobacter cloacae, Citrobacter sp., Proteus sp., Serratia sp., Yersinia sp., Acinetobacter sp., Actinobacillus sp. Bordetella sp., Brucella sp., Capnocytophaga sp., Cardiobacterium sp., Eikenella sp., Francisella sp., Haemophilus sp., Kingella sp., Pasteurella sp., Flavobacterium sp. Xanthomonas sp., Burkholderia sp., Aeromonas sp., Plesiomonas sp., Legionella sp. and alpha-proteobacteria such as Wolbachia sp., cyanobacteria, spirochaetes, green sulfur and green non-sulfur bacteria, Gram-negative cocci, Gram negative bacilli which are fastidious, Enterobacteriaceae-glucose-fermenting Gram-negative bacilli, Gram negative bacilli—non-glucose fermenters, Gram negative bacilli—glucose fermenting, oxidase positive.

In one embodiment of the present invention, the E. coli host strain C41(DE3) is used, because this strain has been previously optimized for general membrane protein overexpression (Miroux et al., “Over-production of Proteins in Escherichia coli: Mutant Hosts That Allow Synthesis of Some Membrane Proteins and Globular Proteins at High Levels,” J Mol Biol 260:289-298 (1996), which is hereby incorporated by reference in its entirety). Further optimization of the host strain includes deletion of the gene encoding the DnaJ protein (e.g., ΔdnaJ cells). The reason for this deletion is that inactivation of dnaJ is known to increase the accumulation of overexpressed membrane proteins and to suppress the severe cytotoxicity commonly associated with membrane protein overexpression (Skretas et al., “Genetic Analysis of G Protein-coupled Receptor Expression in Escherichia coli: Inhibitory Role of DnaJ on the Membrane Integration of the Human Central Cannabinoid Receptor,” Biotechnol Bioeng (2008), which is hereby incorporated by reference in its entirety). Applicants have observed this following expression of Alg1 and Alg2. Furthermore, deletion of competing sugar biosynthesis reactions may be required to ensure optimal levels of N-glycan biosynthesis. For instance, the deletion of genes in the E. coli 0 antigen biosynthesis pathway (Feldman et al., “The Activity of a Putative Polyisoprenol-linked Sugar Translocase (Wzx) Involved in Escherichia coli O Antigen Assembly is Independent of the Chemical Structure of the O Repeat,” J Biol Chem 274:35129-35138 (1999), which is hereby incorporated by reference in its entirety) will ensure that the bactoprenol-GlcNAc-PP substrate is available for other reactions. To eliminate unwanted side reactions, the following are representative genes that may be deleted from the E. coli host strain: wbbL, glcT, glf; gafT, wzx, wzy, waaL, nanA, wcaJ.

Methods for transforming/transfecting host cells with expression vectors are well-known in the art and depend on the host system selected, as described in Sambrook et al., Molecular Cloning: A Laboratory Manual, Cold Springs Laboratory Press, Cold Springs Harbor, N.Y. (1989). For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation, and transfection using bacteriophage.

One aspect of the present invention is directed to a glycoprotein conjugate comprising a protein and at least one peptide comprising a D-X₁-N-X₂-T motif fused to the protein, wherein D is aspartic acid, X₁ and X₂ are any amino acid other than proline, N is asparagine, and T is threonine.

Various host cells can be used to recombinantly produce PSA. In select embodiments, host cells are genetically modified to remove the existing native glycosyltransferases and are engineered to express the glycosyltransferases of the invention for PSA production. To remove the existing glycosylation, host cells are engineered to express endoglycosidase or amidase that cleave between the innermost GlcNAc and asparagine residues of high mannose, hybrid, and complex oligosaccharides from N-linked glycoproteins. Since glycosylation is essential, one may not be able to entirely eliminate the native glycan. In other embodiments, sialic acid bearing glycans may be engineered in the host cell and used as substrates for polysialiation such as ST8Sia II, ST8Sia IV, or NeuS to transfer multiple α2-8 sialic acids to acceptor N-glycans.

In preferred aspects, the invention provides methods for recombinant production of various glycoproteins in vivo. In one embodiment, PSA-conjugated glucagon peptide is produced in glycoengineered E. coli. Using a glycosylation tag (GlycTag) [PCT/US2009/030110], glucagon peptide from glycoengineered E. coli harboring the PSA genetic machinery is expressed and purified. Conjugation of PSA is confirmed by Western blot analysis using commercially available anti-PSA antibodies.

Alternative Expression Systems

Use of eukaryotic expression systems such as mammalian, yeast, fungi, plant or insect cells can be employed to produce PSA-conjugated proteins. In these embodiments, native glycosylation pathways may be disrupted in order to reduce interference with the engineered glycan pathway.

Production of PSA Using Yeast or Fungal Systems

Expression of a sialyltransferase has been demonstrated in P. pastoris (Hamilton, et al, “Humanization of Yeast to Produce Complex Terminally Sialylated Glycoproteins”, Science, vol. 313, pp. 1441-1443 (2006)). By amplifying the E. coli neuA, neuB and neuC genes, a pool of CMP-sialic acid was shown to accumulate in yeast. Yeast or other fungal systems are suitable expression hosts to express the various glycosyltransferases for the production of human antigens or PSA.

Expressing PSA Operon in Plant Cell, e.g., Tobacco, Lemna or Algae

As described in the U.S. Pat. No. 6,040,498, lemna (duckweed) can be transformed using both agrobacterium and ballistic methods. Using protocols described, lemna is transformed and the resulting oligosaccharide composition is transferred onto a target protein. Transgenic plants can be assayed for those that produce proteins with desired human antigens or PSA residues according to known screening techniques.

Production of PSA Using Insect Cell Systems

The present invention can also be applied to the metabolically transformed cell lines derived from Sf9 cells. Sf9 has been used as a production host for recombinant proteins such as interferons, IL-2, plasminogen activators among others, based on its relative ease at which proteins are cloned, expressed and purified in comparision to mammalian cells. Sf9 more readily accepts foreign genes coding for recombinant proteins than many vertebrate animal cells because it is very receptive to viral infection and replication [Bishop, D. H. L. and Possee, R. D., Adv. Gene Technol., 1, 55, (1990)]. Expression levels of recombinant proteins are extremely high in Sf9 and can approach 500 mg/liter [Webb, N. R. and Summers, M. D., Technique, 2, 173 (1990)]. The cell line performs a number of key post-translational modifications; however, they are not identical to those in vertebrates and, therefore, may alter protein function [Fraser, M. J., In Vitro Cell. Dev. Biol., 25, 225 (1989)]. Despite this, the majority of recombinant proteins that undergo post-translational modification in insect cells are immunologically and functionally similar to their native counterparts [Fraser, M. J., In Vitro Cell. Dev. Biol., 25, 225 (1989)]. In contrast to animal cell culture, Sf9 facilitates protein purification by expressing relatively low levels of proteases and having a high ratio of recombinant to native protein expression [Goswami, B. B. and Glazer, R. O. BioTechniques, 10, 626 (1991)].

Baculoviruses serve as expression systems for the production of recombinant proteins in insect cells. These viruses are pathogenic towards specific species of insects, causing cell lysis [Webb, N. R. and Summers, M. D., Technique, 2, 173 (1990)].

Recombinant protein expression in insect cells is achieved by viral infection or stable transformation. For the former, the desired gene is cloned into baculovirus at the site of the wild-type polyhedron gene [Webb, N. R. and Summers, M. D., Technique, 2, 173 (1990); Bishop, D. H. L. and Possee, R. D., Adv. Gene Technol., 1, 55, (1990)]. The polyhedron gene is nonessential for infection or replication of baculovirus. It is the principle component of a protein coat in occlusions which encapsulate virus particles. When a deletion or insertion is made in the polyhedron gene, occlusions fail to form. Occlusion negative viruses produce distinct morphological differences from the wild-type virus. These differences enable a researcher to identify and purify a recombinant virus. In baculovirus, the cloned gene is under the control of the polyhedron promoter, a strong promoter which is responsible for the high expression levels of recombinant protein that characterize this system. Expression of recombinant protein typically begins within 24 hours after viral infection and terminates after 72 hours when the Sf9 culture has lysed.

Stably-transformed insect cells provide an alternate expression system for recombinant protein production [Jarvis, D. L., Fleming, J.-A. G. W., Kovacs, G. R., Summers, M. D., and Guarino, L. A., Biotechnology, 8, 950 (1990); Cavegn, C., Young, J., Bertrand, M., and Bernard, A. R., in Animal Cell Technology: Products of Today, Prospects for Tomorrow, Spier, R. E., Griffiths, J. B., and Berthold, W., Eds. (Butterworth-Heinemann, Oxford, 1994, pp. 43-49)]. In these cells, the desired gene is expressed continuously in the absence of viral infection. Stable transformation is favored over viral infection when recombinant protein production requires cellular processes that are compromised by the baculovirus. This occurs, for example, in the secretion of recombinant human tissue plasminogen activator from Sf9 cells [Jarvis, D. L., Fleming, J.-A. G. W., Kovacs, G. R., Summers, M. D., and Guarino, L. A., Biotechnology, 8, 950 (1990)]. Viral infection is favored when the recombinant protein is cytotoxic since protein expression is transient in this system.

Insect cells for in vitro cultivation have been produced and several cell lines are commercially available. This process includes using insect cells capable of culture as described herein regardless of the source. The preferred cell line is Lepidoptera Sf9 cells. Other cell lines include Drosophila cells from the European Collection of Animal Cell Cultures (Salisbury, UK) or cabbage looper Trichoplusia ni cells including High Five available from Invitrogen Corp. (San Diego, Calif.) Sf9 insect cells from either Invitrogen Corporation or American Type Culture Collection (Rockville, Md.) are the preferred cell line and were cultivated in the bioreactor freely suspended in serum-free EX-CELL 401 Medium purchased from JRH Biosciences (Lenexa, Kans.) and maintained at 27° C.

Oligosaccharide Compositions

The prokaryotic system can yield homogenous glycans at a relatively high yield. In preferred embodiments, the oligosaccharide composition comprises or consists essentially of a single glycoform in at least 50, 60, 70, 80, 90, 95, 99 mole %. In further embodiments, the oligosaccharide composition consists essentially of two desired glycoforms of at least 50, 60, 70, 80, 90, 95, 99 mole %. In yet further embodiments, the oligosaccharide composition consists essentially of three desired glycoforms of at least 50, 60, 70, 80, 90, 95, 99 mole %. The present invention, therefore, provides stereospecific biosynthesis of a vast array of novel oligosaccharide compositions and N-linked glycoproteins including glycans for BGA and PSA.

Select PSA oligosaccharide compositions include:

(Sia α2,8)ₙ-Sia α2,8-Sia α2,3-Galβ1,3-GalNAc α1,3-GalNAc α1,3-GlcNAc; (Sia α2,8)ₙ-Sia α2,8-Sia α2,3-Galβ1,3-GalNAc α1,3-GlcNAc; (Sia α2,8)ₙ-Sia α2,8-Sia α2,3-Galβ1,3-(GalNAc α1,3)ₙ.

Select Sialyl T Antigen oligosaccharide compositions include:

Sia α2,3-Galβ1,3-GalNAc α1,3-GlcNAc; Sia α2,3-Galβ1,3-GalNAc α1,3-GalNAc α1,3; Sia α2,3-Galβ1,3-GalNAc α1,3-

Select H Antigen oligosaccharide compositions include:

Fuc α1,2-Galβ1,3-GalNAc α1,3-GlcNAc; Fuc α1,2-Galβ1,3-GalNAc α1,3-GalNAc α1,3; Fuc α1,2-Galβ1,3-GalNAc α1,3

Select T Antigen oligosaccharide compositions include:

Galβ1,3-GalNAc α1,3-GlcNAc; and Galβ1,3-GalNAc α1,3-GalNAc α1,3.

Other select PSA oligosaacharide compositions include:

[βGlcNAc][βGalNAc][βGalNAc][β1,4Gal] [α(2→3)Neu5Ac]ₙ; [α(2→6)Neu5Ac]ₙ; [α(2→8)Neu5Ac]ₙ or [α(2→9)Neu5Ac]ₙ.

Target Glycoproteins

Various examples of suitable target glycoproteins may be produced according to the invention, which include without limitation: cytokines such as interferons, G-CSF, coagulation factors such as factor VIII, factor IX, and human protein C, soluble IgE receptor α-chain, IgG, IgG fragments, IgM, interleukins, urokinase, chymase, and urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, α-feto proteins, AAT, rhTBP-1 (aka TNF binding protein 1), TACI-Ig (transmembrane activator and calcium modulator and cyclophilin ligand interactor), FSH (follicle stimulating hormone), GM-CSF, glucagon, glucagon peptides, GLP-1 w/ and w/o FC (glucagon like protein 1) IL-1 receptor agonist, sTNFr (aka soluble TNF receptor Fc fusion), CTLA4-Ig (Cytotoxic T Lymphocyte associated Antigen 4-Ig), receptors, hormones such as human growth hormone, erythropoietin, peptides, stapled peptides, human vaccines, animal vaccines, serum albumin and enzymes such as ATIII, rhThrombin, glucocerebrosidase and asparaginase.

Antibodies, fragments thereof and more specifically, the Fab regions such as adalimumab, atorolimumab, fresolimumab, golimumab, lerdelimumab, metelimumab, morolimumab, sifalimumab, ipilimumab, tremelimumab, bertilimumab, briakinumab, canakinumab, fezakinumab, ustekinumab, adecatumumab, belimumab, cixutumumab, conatumumab, figitumumab, intetumumab, iratumumab, lexatumumab, lucatumumab, mapatumumab, necitumumab, ofatumamb, panitumumab, pritumumab, rilotumumab, robatumumab, votumumab, zalutumumab, zanolimumab, denosumab, stamulumab, efungumab, exbivirumab, foravirumab, libivirumab, rafivirumab, regavirumab, sevirumab, tuvirumab, nebacumab, panobacumab, raxibacumab, ramucirumab, gantenerumab.

Full-length monoclonal antibodies have traditionally been produced in mammalian cell culture due to their parental hybridoma source, the complexity of the molecule, and the desirability of glycosylation of the monoclonal antibodies. Generally, Escherichia coli is the host system of choice for the expression of antibody fragments such as Fv, scFv, Fab or F(ab′)₂. These fragments can be made relatively quickly in large quantities with the retention of antigen binding activity. However, because antibody fragments lack the Fc domain, they do not bind the FcRn receptor and are cleared quickly. Full-length antibody chains can also be expressed in E. coli as insoluble aggregates and then refolded in vitro, but the complexity of this method limits its usefulness. Accordingly, the antibodies are produced in the periplasm.

In contrast to the widespread uses of bacterial systems for expressing antibody fragments, there have been few attempts to express and recover at high yield functional intact antibodies in E. coli. Because of the complex features and large size of an intact antibody, it is often difficult to achieve proper folding and assembly of the expressed light and heavy chain polypeptides, which results in poor yield of reconstituted tetrameric antibody. Furthermore, antibodies made in prokaryotes are not glycosylated. Since glycosylation is required for Fc receptor mediated activity, it is conventionally considered that E. coli would not be a useful system for making intact antibodies. (Pluckthun and Pack (1997) Immunotech 3:83-105; Kipriyanov and Little (1999) MoI. Biotech. 12:173-201). Recombinant oligosaccharide synthesis changes this paradigm.

Recent developments in research and clinical studies suggest that in many instances, intact antibodies are preferred over antibody fragments. An intact antibody containing the Fc region tends to be more resistant to degradation and clearance in vivo, thereby having longer biological half life in circulation. This feature is particularly desirable where the antibody is used as a therapeutic agent for diseases requiring sustained therapies.

Currently, anti-TNF antibodies are produced in mammalian cells and are glycosylated. The cost of producing antibodies in mammalian cells (frequently in CHO cells) is high and the procedure is complex. Glycosylation of antibodies has two effects: first, it can increase the lifetime of the antibody in the blood serum, so that it circulates for many days or even weeks. This may be because of decreased kidney clearance or because of greater resistance to proteolysis. Second, as provided herein, glycosylation in the constant region of the antibody is important for activating the “effector functions” of the antibody, which are triggered when an antibody binds to a target that is attached to a cell surface. These functions are linked to activation of the immune system and can lead to natural killer (NK) mediated cell killing.

Pharmaceutical Compositions and Pharmaceutical Administration

Another aspect of the invention is a composition as defined above which is a pharmaceutical composition and further comprises one or more pharmaceutically acceptable excipients. The pharmaceutical composition may be in the form of an aqueous suspension. Aqueous suspensions contain the novel compounds in admixture with excipients suitable for the manufacture of aqueous suspensions. The pharmaceutical compositions may be in the form of a sterile injectable aqueous or homogeneous suspension. This suspension may be formulated according to the known art using suitable dispersing or wetting agents and suspending agents.

Pharmaceutical compositions may be administered orally, intravenously, intraperitoneally, intramuscularly, subcutaneously, intranasally, intradermal, topically or intratracheal for human or veterinary use.

The protein, peptide, antibody and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances or minor amounts of auxiliary substances such as wetting or emulsifying agents, preservatives or buffers, which enhance the shelf life or effectiveness of the protein, peptide, antibody or antibody portion.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., protein, peptide, antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The protein, peptide, antibody and antibody-portions of the present invention can be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

The above disclosure generally describes the present invention. A more specific description is provided below in the following examples. The examples are described solely for the purpose of illustration and are not intended to limit the scope of the present invention. Changes in form and substitution of equivalents are contemplated as circumstances suggest or render expedient. Although specific terms have been employed herein, such terms are intended in a descriptive sense and not for purposes of limitation.

### Example 1

### Plasmid Construction

Plasmids in this study were constructed using standard homologous recombination in yeast (Shanks R M, Caiazza N C, Hinsa S M, Toutain C M, O'Toole G A: Saccharomyces cerevisiae-based molecular tool kit for manipulation of genes from gram-negative bacteria. Appl Environ Microbiol 2006, 72(7):5027-5036)). Plasmids were recovered from yeast and transferred to E. coli strain DH5α for confirmation via PCR and/or sequencing. The following list describes plasmids constructed during the course of this study. The plasmid name is followed by the inserted genes/sequences in order from 5′-3′ followed by the vector in parentheses. All glycan expression plasmids were constructed in vector pMW07 (Vaderrama-Rincon et al.). Protein expression plasmids were constructed in vector pTRCY. Sugar nucleotide synthesis plasmids were cloned in pTrcY, pMW70.

In order of figures:

pMW07: (vector) pBAD, Chlor R (pMW07) Valderrama et al

pDis-07: galE, pglB, pglA (pMW07)

pDisJ-07: galE, pglB, pglA, wbnJ (pMW07)

pMBP-hGH-Y: malE (no signal sequence)-hexahistidine-tev-hGH (pTrcY) (“hexahistidine” disclosed as SEQ ID NO: 36)

pscFv13 4XDQNAT-Trc99a: ssdsbA-scFv13-4×GlycTag-hexahistidine (pMW07) Valderrama et al (“DQNAT” disclosed as SEQ ID NO: 37 and “hexahistidine” disclosed as SEQ ID NO: 36)

pDisJ-07: galE, pglB, pglA, wbnJ (pMW07)

pMG1X-Y: ssdsbA-malE-glucagon-4×GlycTag-hexahistidine (pTrcY) (“hexahistidine” disclosed as SEQ ID NO: 36)

pJDLST-07: galE, pglB, pglA, neuD, neuB, neuA, neuC, 1st, wbnJ (pMW07)

pMG1X NeuDBAC-Y: ssdsbA-malE-glucagon-1×GlycTag-hexahistidine (“hexahistidine” disclosed as SEQ ID NO: 36), NeuDBAC (pTrcY)

pJCstIIS-07: galE, pglB, pglA, neuS, neuB, neuA, neuC, cstI1260, wbnJ (pMW07)

pJLic3BS-07: galE, pglB, pglA, neuS, neuB, NeuA, neuC, Lic3B, wbnJ (pMW07)

pMBP-3TEV-GLUC-4XGlycTag-6H-Y: ssdsbA-malE-glucagon-4×GlycTag-hexahistidine (pTrcY) (“6H” and “hexahistidine” disclosed as SEQ ID NO: 36)

pNeuD-Y: neuD (pTrcY)

pMBP4X-Y: ssdsbA-malE-4×GlycTag-hexahistadine (pTrcY) (“hexahistidine” disclosed as SEQ ID NO: 36)

pCstII*SiaD-Y: cstII153S260-siaD (pTrcY)

pCstIISiaD-Y: cstII260-siaD (pTrcY)

pJK-07: galE, pglB, pglA, wbnJK (pMW07)

pGNF-70: galE(Cj), galE(K12), gmd, fcl, gmm, cpsBG (pMQ70)

pMG1×KGNF-Y: ssdsbA-malE-glucagon-IX GlycTag-hexahistidine galE(K12) (“hexahistidine” disclosed as SEQ ID NO: 36), wbnK, gmd, fcl, gmm, cpsBG (pTrcY)

Strains (in order of figures)

MC4100

MC4100 ΔwaaL

MC4100 ΔwaaL ΔnanA

MC4100 ΔnanA

LPS1 ΔwaaL

LPS1

E. coli MC4100 was selected as a host for functional testing because it does not natively express glycan structures containing sialic acid and it has served as a functional host for glycosylation previously (Vaderrama-Rincon et al. “An engineered eukaryotic protein glycosylation pathway in E. coli,” Nat Chem Bio 8, 434-436 (2012)). The mutations in the waaL, and nanA genes were transduced from the corresponding mutant in the Keio collection. The kan cassette was later removed from the MC4100 ΔnanA strain. Mutations generated in the K1 E. coli background used the method of Datsenko and Wanner. Mutations in the kpsS and neuS were made by transforming with a kan cassette flanked by appropriate regions of homology near the 5 and 3′ ends of the respective genes (Datsenko et al.). For surface expression of glycans, plasmids of interest were used to transform MC4100, MC4100ΔnanA, MC4100ΔnanA waaL::kan or LPS1 E. coli. Protein glycosylation experiments were performed in strains as indicated with pTrc-ssDsbA-R4-GT encoding scFvl3-R4 modified with a C-terminal GlycTag and hexahistidine tag (SEQ ID NO: 36) (R4-GT-6H) (“6H” disclosed as SEQ ID NO: 36).

Media and Reagents

Antibiotic selection was maintained at: 100 μg/mL ampicillin (Amp), 25 μg/mL chloramphenicol (Chlor), 10 ug/mL tetracycline (Tet) and 50 μg/mL kanamycin (Kan). Routine growth of E. coli cultures was performed in LB medium supplemented with glucose at 0.2% and antibiotics as necessary. For expression of PSA plasmids, LB medium was supplemented with sialic acid (Sigma or Millipore) at a final concentration of 0.25% and the medium was adjusted to pH ˜7.5 and sterilized. Plasmids for glycan and protein expression were induced with the addition of L-arabinose at 0.2% or isopropyl β-d-thiogalactoside (IPTG) at 100 mM respectively. Yeast FY834 was maintained on YPD medium and synthetic defined—Uracil medium was used to select or maintain yeast plasmids.

Cell-Surface Glycan Detection

Dot blots were performed using 2.5 μl or 4 μl of overnight LB culture from strain indicated. Cells were spotted on a nitrocellulose membrane and PSA glycans were detected by immunoblot as below. Phage susceptibility testing was performed using agar overlays containing the strain of interest. 2 μl of PSA specific Phage F was spotted on the overlay and plates were incubated at 37° C. overnight. For flow cytometry cultures were inoculated in LB supplemented with antibiotics as appropriate. The medium also included sialic acid at a final concentration of 0.25% and 0.2% arabinose. Cells were harvested ˜18 hours post-induction, resuspended in PBS, heated to 95° C. for 10 minutes and cooled to room temperature prior to incubation with the anti-PSA antibody followed by goat anti-IgM-FITC. Analysis was performed using a BD FACScalibur flow cytometer.

Protein Expression and Purification

Strains to be harvested for analysis of N-glycosylation were inoculated into LB with the appropriate antibiotics and incubated with shaking at 30° C. until the cultures reached an OD₆₀₀ of 2-3. Plasmids for glycan expression were induced with the addition of arabinose and production of the acceptor protein was induced with IPTG. Cultures were harvested 16-18 h post induction. Cell lysis and purification of glycoproteins was performed using the Ni-NTA kit (Qiagen).

Protein Analysis

Proteins were separated by SDS-polyacrylamide gels (Lonza), and Western blotting was performed as described previously (DeLisa M P, et al., Folding quality control in the export of proteins by the bacterial twin-arginine translocation pathway. Proc Natl Acad Sci USA 2003, 100(10):6115-6120). Briefly, proteins were transferred onto polyvinylidene fluoride (PVDF) membranes and membranes were probed with one of the following: anti-6×-His (SEQ ID NO: 36) antibodies conjugated to HRP (Sigma), or anti-PSA-NCAM (Millipore). In the case of the anti-PSA antiserum, anti-mouse IgG-HRP (Promega) was used as the secondary antibody.

### Example 2

### Engineering E. coli for Expression of the Human Thomsen-Friedenreich Antigen (T-antigen)

In order to assemble a glycan containing the human Thomsen-Friedenreich antigen (T-antigen, Galβ1,3 GalNAca-) in E. coli, a plasmid was constructed for expression of the glycosyltransferase and sugar nucleotide epimerase activities necessary to produce this structure using the native UndPP-GlcNAc as a substrate. Plasmid pMW07 (Valderrama-Rincon et al.) was used as the vector because it contains a low copy number origin of replication (ORI), an inducible pBAD promoter, and a yeast ORI allowing for cloning via homologous recombination in Saccharomyces cerevisiae. The sequence of pMW07 is provided as SEQ ID NO: 1.

To generate a disaccharide glycan with the structure GalNAcα1,3 GlcNAc, a plasmid was constructed to express the C. jejuni GalNAc transferase PglA, and the epimerase GalE to promote synthesis of the UDP-GalNAc substrate. The gene encoding the OST PglB from C. jejuni was also included for use in glycosylation in the future. A PCR fragment including galE, pglB, and pglA along with linearized pMW07 was used to co-transform S. cerevisiae and cloning was performed by homologous recombination in yeast as previously described (Shanks et al.). Plasmid was isolated from colonies selected on synthetic defined—uracil medium and used to transform E. coli DH5a for confirmation of construct. The resulting plasmid was designated pDis-07.

The human Thomsen-Friedenreich or T-antigen glycan consists of Ga1131-3GalNAca structure. Galactose transferase WbnJ from E. coli 086 was selected as the glycosyltransferase to incorporate the terminal galactose residue because it is reported to attach galactose in a β1,3 linkage to a GalNAc residue and is a native bacterial enzyme (Yi W, Shao J, Zhu L, Li M, Singh M, Lu Y, Lin S, Li H, Ryu K, Shen J et al: Escherichia coli O86 O-Antigen Biosynthetic Gene Cluster and Stepwise Enzymatic Synthesis of Human Blood Group B Antigen Tetrasaccharide. Journal of the American Chemical Society 2005, 127(7):2040-2041). The wbnJ gene was amplified from a synthetic plasmid from Mr. Gene and homologous recombination in yeast was used to combine the resulting PCR product and linearized pDis-07 plasmid. The resulting plasmid is named pDisJ-07 and contains the following genes as a synthetic operon under control of a pBAD promoter: (5′-3′) galE, pglB, pglA, wbnJ.

In their native context, the substrates for both glycosyltransferases PglA and WbnJ are saccharides assembled on the lipid undecaprenylpyrophosphate (UndPP). As part of the E. coli K12 LPS synthesis pathway, a GlcNAc residue is first added to UndPP via the activity of native WecA and the resulting GlcNAc is then transferred to the lipid A core oligosaccharide in the periplasm by the WaaL ligase. Finally, the lipid A moiety is transported to the outer membrane resulting in cell-surface display of the glycans. Cells carrying deletions in the waaL gene are unable to transport UndPP-linked glycans to the cell surface and thus, this mutation is useful for confirming that a glycan is linked to UndPP.

The waaL (rfaL) gene has been previously mutated as part of the Keio collection and the resulting strain rfaL734(del)::kan (JW3597-1) (Baba T, Ara T, Hasegawa M, Takai Y, Okumura Y, Baba M, Datsenko K A, Tomita M, Wanner B L, Mori H: Construction of Escherichia coli K-12 in-frame, single-gene knockout mutants: the Keio collection. Mol Syst Biol 2006, 2) was obtained from the Yale Coli Genetic Stock Center (CGSC). P1 vir phage was used to transduce the waaL mutation into an MC4100 recipient to make strain MC4100 ΔwaaL::kan. Plasmid pCP20 was used to then remove the Kan cassette (Datsenko K A, Wanner B L: One-step inactivation of chromosomal genes in Escherichia coli K-12 using PCR products. Proceedings of the National Academy of Sciences 2000, 97(12):6640-6645) resulting in strain MC4100 ΔwaaL.

Flow cytometry was used to analyze the cell surface glycans produced by E. coli MC4100 expressing pDisJ-07 to confirm the presence of a galactose-terminal structure compared to control plasmid pDis-07. Cultures were inoculated in 1.5 mL tubes containing 1000 μl LB supplemented with 25 μg/μl chloramphenicol and 0.2% arabinose. After a 24 hour incubation shaking at 30° C. the cultures were pelleted and resuspended in 200 μl PBS. 100 μl aliquots of each were heated at 95° C. for 10 minutes and cooled to room temperature. 400 μl PBS was added to each sample and 3 μl of fluorescein labeled Soy Bean Agglutinin (SBA, Vector laboratories) or Ricinus Communis Agglutinin I (RCA I, vector laboratories) which preferentially binds to galactose terminal glycans. Samples were incubated on a rocking platform at room temperature for 10 minutes in the dark prior to flow cytometry.

Flow cytometry with the RCA I lectin was suggests the presence of a galactose terminal glycan on the cell surface of MC4100 cells expressing pDisJ-07 but not pDis-07 (FIG. 2, left). This result is consistent with the previously reported function of the WbnJ enzyme as a galactosyl transferase. Cell-surface labeling with SBA-fluorescein (FIG. 2, center) was reduced in cells expressing the pDisJ-07 plasmid compared to the pDis-07 plasmid suggesting a reduction in the amount of available terminal GalNAc residues. In a MC4100 ΔwaaL mutant, fluorescence was greatly reduced for cells expressing either plasmid suggesting that these are both synthesized as UndPP-linked glycans.

### Example 3

### In Vivo Synthesis of Proteins Carrying an N-Glycan Terminating in the Human T Antigen

The OST PglB is utilized to transfer UndPP-linked oligosaccharides to specific asparagine residues. This requires a target protein bearing the PglB recognition site consisting of the DXNXT sequon to be localized to the periplasm and the presence of an appropriate glycan substrate. For this study, we also constructed vector pTRCY for use in expression of glycoproteins.

pTRCY was cloned via homologous recombination in S. cerevisiae by adding the URA3 gene and the yeast 2 micron ORI to pTRC99a thus generating a novel vector capable of replicating in yeast. The URA3 gene and 2 micron ORI were amplified with primers containing homology to vector pTRC99a between the pBR322ORI and lacI gene.

hGH was cloned as a c-terminal translational fusion following a signal peptide from E. coli DsbA, MBP, hexahistidine tag (SEQ ID NO: 36), and a tev cleavage site. The hGH gene was further modified to contain a single glycosylation acceptor site DQNAT (SEQ ID NO: 37) and the final construct is named pMBP-hGH-Y.

Strains MC4100ΔnanAΔwaaL bearing plasmids pDisJ-07 and pMBP-hGH-Y or pMBP-hGH-Y alone are grown under ampicillin (100 μg/μl) and chloramphenicol (25 μg/μl) or ampicillin (100 μg/μl) selection respectively. pDisJ-07 is induced with the addition of 0.2% (v/v) arabinose and IPTG is added after approximately 16 h to induce protein production. The protein was partially purified by nickel affinity chromatography and treated with TEV protease (Sigma) to release hGH prior to analysis by SDS-PAGE and Coomassie staining. The visible mobility shift in the presence of the pDisJ-07 plasmid is consistent with glycosylation (FIG. 2, right).

### Example 4

### Confirm Identity and Linkage of the Galactose Residue in the Human T Antigen

To further probe the identity of the glycan produced upon expression of pDisJ-07, we extracted the lipid-linked oligosaccharides and analyzed the released glycans by mass spectrometry. A 1:100 inoculum was use to seed 4 250 mL cultures containing LB supplemented with 25 μg/μl chloramphenicol. Cultures were grown at at 30° C. and induced when the ABS₆₀₀ reached ˜2.0. Cells were harvested after ˜20 hours for isolation of lipid-linked oligosaccharides by the method of Gao and Lehrman (Gao N, Lehrman M: Non-radioactive analysis of lipid-linked oligosaccharide compositions by fluorophore-assisted carbohydrate electrophoresis. Methods Enzymol 2006, 415:3-20). Briefly, pellet was resuspended in 10 mL methanol and lysed by sonication. Material was dried at 60° C. and subsequently resuspended in 1 mL 2:1 chloroform:methanol (v/v, CM) via sonication and material was washed two times in CM. The pellet was then washed in water then lipids were extracted with 10:10:3 chloroform:methanol: water (v/v/v, CMW) followed by methanol. The CMW and methanol extracts were combined and loaded onto a DEAE cellulose column. CMW was used to wash the column and lipid-linked oligosaccharides were eluted with 300 mM NH₄OAc in CMW. The lipid-linked oligosaccharides were extracted with chloroform and dried.

To release the glycans from the lipids, the material was resuspended in 1.5 mL 0.1N HCl in 1:lisopropanol: water (v/v). The solution was heated at 50° C. for 2 hours and then dried at 75° C. Residue was suspended in water saturated butanol and the aqueous phase containing the glycans was dried, resuspended in water, and purified with AG50W-H8(hydrogen atom) cation exchange resin followed by Agl-X8 (formate form) anion exchange resin.

Purified oligosaccharides were analyzed on an AB SCIEX TOF/TOF mass spectrometer using dihydroxybenzoic acid (DHB) as the matrix (FIG. 3). A predominant peak was consistent with the desired glycoform Gal GalNAc GlcNAc (m/z 609). To confirm the identity of the terminal glycan, the sample was divided and half was treated with β1,3 galactosidase (NEB) and half with a water control. Samples were incubated at 37° C. for 48 hours and analyzed by mass spectrometry revealing a major peak at (m/z 447) consistant with the expected size of the disaccharide GalNAc GlcNAc.

### Example 5

### Engineering E. coli for Expression of the Human Sialyl-T Antigen

The human sialyl-T antigen consists of the T antigen glycan modified with a terminal α2,3 Neuraminic acid (NeuNAc) residue resulting in the following structure: NeuNAcα2,3 Gal β1,3 GalNAcα-. To generate a glycan terminating with the sialyl T antigen structure in an E. coli host, the plasmid described above expressing genes required to synthesize the T-antigen glycan (pDisJ-07) was modified to include a gene encoding a sialyltransferase, and genes whose products comprise the cytidine 5′ monophospho-N-acetylneuraminic acid (CMP-NeuNAc) synthesis pathway in E. coli K1.

A region of DNA was amplified from the E. coli K1 genome including the genes neuB, neuA, and neuC using PCR. These encode a Neu5Ac synthase, CMP-Neu5Ac synthetase, and UDP-GlcNAc2-epimerase respectively. The neuD gene was also included as it may help to stabilize the neuB gene product (Daines D A, Wright L F, Chaffin D O, Rubens C E, Silver R P: NeuD plays a role in the synthesis of sialic acid in Escherichia coli K1. FEMS microbiology letters 2000, 189(2):281-284). The lst gene encoding the N. meningitidis α2,3 sialyltransferase was also amplified and both PCR products along with linearized pDisJ-07 were used to co-transform S. cerevisiae to make resulting plasmid pJDLST-07 by homologous recombination. Plasmid pJDLST-07 contains a synthetic operon under control of the pBAD promoter with genes in the following order: galE, pglB, pglA, neuD, neuB, neuA, neuC, lst, wbnJ.

For use in expressing sialylated glycans, a strain was constructed in which the nanA gene encoding the sialic acid aldolase NanA was targeted for disruption. Deletion of the nanA gene prevents degradation of sialic acid from external sources (Vimr E R, Troy F A: Identification of an inducible catabolic system for sialic acids (nan) in Escherichia coli. J Bacteriol 1985, 164(2):845-853). The ΔnanA::kan mutation was introduced into MC4100 E. coli via P1 vir phage transduction from the corresponding mutant generated as part of the Keio collection (CGSC #10423, Yale genetic stock center) (Baba et al.). The kanamycin cassette was removed by the method of Datsenko and Wanner (Datsenko et al.). To promote glycosylation, the ΔwaaL::kan mutation was subsequently introduced and cured of kanamycin resistance by the same method as described above.

### Example 6

### In Vivo Synthesis of Proteins Carrying an N-Glycan Terminating in the Human (2,3)-Sialyl-T Antigen

To permit analysis of sialylated glycopeptide by Mass spectrometry, a Glucagon peptide modified with a 1×GlycTag containing a DQNAT motif (SEQ ID NO: 37) was cloned. To construct this plasmid, the DsbA signal peptide sequence and the malE gene (which encodes MBP) were amplified with primers containing homology to vector pTRCY and the sequence for the TEV protease sites. Similarly, glucagon was amplified from a synthetic oligonucleotide with primers containing sequence encoding the TEV protease site or the sequence for the 4×GlycTag and 6×-His tag (SEQ ID NO: 36) followed by homology to pTRCY. These PCR products were used with linearized pTRCY to co-transform S. cerevisiae for cloning by homologous recombination to generated plasmid pMG4X-Y. The related plasmid pMG1X-Y is a derivative of pMG4X-Y made by replacing the 4XGlycTag with a 1×GlycTag. Briefly, pMG4X-Y was linearized and an oligonucleotide encoding the 1×GlycTag was used to replace the 4XGlycTag by homologous recombination in S. cerevisiae. The sequence encoding proteins MBP-3TEV-GLUC-4XGlycTag-6H (“6H” disclosed as SEQ ID NO: 36) and MBP-3TEV-GLUC-IXGlycTag-6H (“6H” disclosed as SEQ ID NO: 36) are provided.

In order to generate glycoprotein in vivo containing the human sialyl-T antigen, strain MC4100ΔnanA ΔwaaL described above was used to promote periplasmic accumulation of sialylated glycans. This strain was co-transformed with plasmid pMG1X-Y encoding a glycosylation acceptor protein and pJDLST-07 which expresses the machinery necessary to synthesize the sialyl-T antigen glycan.

An overnight culture consisting of MC4100ΔnanA ΔwaaL pMG1X-Y and pJDLST-07 was used to inoculate a 50 mL culture in LB with 100 μg/μl ampicillin and 25 μg/μl chloramphenicol. When the ABS₆₀₀ reached approximately 1.5 the culture was induced with arabinose to 0.2% and IPTG to 1 mM and the cells were harvested by centrifugation approximately 19 hours post-induction. Following cell lysis, protein was purified on a NiNTA column and TEV protease was used to cleave 30 μl of the resulting eluate. The sample was incubated at 30° C. for 3 h and an aliquot was analyzed by mass spectrometry on an AB SCIEX TOF/TOF mass spectrometer using dihydroxybenzoic acid (DHB) as the matrix.

Mass spectrometry revealed major peaks consistent with the expected size of glucagon modified with the sialyl-T antigen (m/z 6251) and the expected size of glycosylated Glucagon bearing the T antigen terminal glycan (m/z 5960) (FIG. 4).

### Example 7

### Relative Improvement of Sialylation Through Expression of neuDBAC from TRCY

One potential strategy for improving sialylation in this system is to increase the intracellular availablilty of CMP-NeuNAc. Although the necessary biosynthetic genes are present on plasmid JDLST-07, it was hypothesized that additional copies could improve sialylation. The genes neuDBAC were amplified as a single PCR product and inserted into pMG1X-Y downstream of the glucagon fusion protein using homologous recombination in Saccharomyces cerevisiae. This resulted in creation of plasmid pMG1X NeuDBAC-Y.

Plasmid pMG1XNeuDBAC-Y was combined with pJDLST-07 in strain MC4100ΔnanA ΔwaaL to test glycosylation in 50 mL cultures as described above. Mass spectrometry of the TEV-cleaved peptide product reveals a major peak consistent with the expected size of glucagon modified with the sialyl-T antigen containing glycan (m/z 6250). A second smaller peak consistent with the expected size of glucagon modified with the T antigen glycan (m/z 5959) is also detected (FIG. 5).

### Example 8

### α2,3 Neuraminidase Treatment of Sialylated Glucagon Peptide

To validate the sialylation of the glucagon peptide a neuramindse treatment was performed. Plasmids pMG1XNeuDBAC-Y and pJDLST-07 in strain MC4100ΔnanA Δwaal is grown in a 50 mL culture in LB with 100 μg/μl ampicillin and 25 μg/μl chloramphenicol. The recombinant protein is purified from the lysate with nickel affinity chromatography and the eluate is buffer exchanged in 50 mM Tris pH 8.0 100 mM NaCl and concentrated prior to incubation for 3 h at 30° C. with TEV protease. The protein is divided and incubated with α2,3 neuraminidase (NEB) or a buffer control for 2 hours at 37° C. prior to analysis by Mass spectrometry (FIG. 6). The major peak in the buffer control sample (m/z 6253) is constant with the expected size of glucagon modified with the siayl-T antigen glycan. In the sialidase treated sample, the major peak (m/z 5961) is consistent with the expected size of the T antigen glycopeptide. No evidence of the sialylated glycopeptide was present following neuraminidase treatment.

### Example 9

### Production of a Recombinantly Produced Polysialylated Glycan in E. coli

There are several bacteria known to produce polysialic acid (PSA) glycans including E. coli K1 and strains of Neisseria meningitidis. In these strains PSA forms a protective capsular polysaccharide. The PSA capsule is well-studied in E. coli K1 but the lipid substrate for PSA synthesis has not been identified. In order to adapt PSA for N-glycosylation, it is likely necessary to direct its synthesis on a substrate appropriate for the OST and provide the necessary disialic acid ‘primer’ required for the PSA polymerase to extend sialylation.

The glycan described herein terminating in the human T antigen is a good candidate for polysialylation because it is efficiently used in glycosylation in this system. To clone a construct for use in exploring polysialylation, a truncated version of the gene cstII encoding the first 260 amino acids of the bifunctional α2,3 α2,8 sialyltransferase, and neuBAC were inserted into the pDisJ-07 plasmid using homologous recombination in Saccharomyces cerevisiae. The full length bifunctional α2,3 α2,8 sialyltransferase lic3b was also cloned in the same manner. The resulting plasmids are called pJCstIIS-07 and pJLic3bS-07.

Plasmid pJCstIIS-07 was used to transform MC4100 ΔnanA and MC4100 ΔnanAΔwaaL for functional testing. A single colony is used to inoculate 1 mL of LB medium containing 25 μg/μl chloramphenicol and 0.2% (v/v) arabinose. Cultures are grown approximately 18 hours at 30° C. in a 1.5 mL tube and the cultures are pelleted. After washing with PBS, cultures are normalized by optical density and heated for 10 min at 95° C. and the whole cells are spotted on nitrocellulose when cooled. The membrane is blotted with an anti-PSA antibody followed by anti-mouse-horseradish peroxidase (FIG. 7a). Reactivity with the PSA antibody suggests that a PSA-glycan is displayed on the cell surface in the presence of waaL. The structure of the expected glycan is diagrammed (FIG. 7b).

To test the putative PSA-terminal glycan in a glycosylation reaction, the MC4100ΔwaalΔnanA strain was transformed with pMG4X-Y encoding a glycosylation acceptor protein. The resulting strain was transformed with plasmid pDisJ-07 or pJLIc3B-07. Resulting strains were grown in 50 mLs LB +/−0.25% NeuNAc and appropriate antibiotics. Cultures are induced at an approximate optical density of 2-4 with 0.2% arabinose and 1 mM IPTG. Proteins were purified by nickel affinity chromatography, concentrated and treated with TEV protease prior to analysis by Western blot (FIG. 8).

Detection with the αPSA antibody (FIG. 8, top) showed some reactive material only in the presence of pJLic3BS-07 and NeuNAc supplementation consistant with presence of a PSA glycan. Total protein is detected by the presence of the hexasitidine tag with αHis antiserum (FIG. 8, bottom).

### Example 10

### NeuD is Important for Synthesis of Sialylated Glycans in E. coli MC4100

In order to confirm the importance of NeuD in the sialylation platform it was cloned as an individual gene into vector pTRCY using homologous recombination in Saccharomyces cerevisiae. The resulting plasmid containing NeuD under the control of the Trc promoter is called pNeuD-Y.

To test pNeuD-Y, this plasmid was used with pJLic3BS-07 to cotransform strain MC4100ΔnanA. A single colony is used to inoculate 1 mL of LB medium containing 25 μg/μl chloramphenicol and 0.2% (v/v) arabinose. LB medium was made with or without sialic acid at a final concentration of 0.25% and was adjusted for pH and filter sterilized. Cultures are grown approximately 18 hours at 30° C. in a 1.5 mL tube and the cultures are pelleted. After washing with PBS, cultures are normalized by optical density and heated for 10 min at 95° C. and the whole cells are spotted on nitrocellulose when cooled. The membrane is blotted with an anti-PSA antibody followed by anti-mouse-horseradish peroxidase (FIG. 9).

Reactivity with the PSA antibody suggests the presence of a cell surface PSA glycan in the presence of pNeuD-Y or NeuNAc. This result suggests the importance of NeuD in production of sialylated compounds in laboratory E. coli (FIG. 9).

### Example 11

### Ex Vivo Polysialylation

As an alternative method to confirm the functionality of polysialyltransferases in laboratory E. coli, an ex vivo method for polysialylation was utilized. For this method a lysate is generated from a strain expressing a polysialyltransferase and it is combined with CMP-NeuNAc and an acceptor protein produced in a separate strain. MBP was selected for use as the acceptor protein because it is expressed and glycosylated efficiently in this system.

To prepare the acceptor protein plasmid, the coding sequence for MBP modified with the DsbA signal peptide and a 4×GlycTag and hexahistidine motif (SEQ ID NO: 36) was subcloned from pTRC99-MBP 4×DQNAT (“DQNAT” disclosed as SEQ ID NO: 37) (Fisher A C, Haitjema C H, Guarino C, celik E, Endicott C E, Reading C A, Merritt J H, Ptak A C, Zhang S, DeLisa M P: Production of Secretory and Extracellular N-Linked Glycoproteins in Escherichia coli. Applied and Environmental Microbiology 2011, 77(3):871-881). The resulting plasmid is termed pMBP4XGT-Y. CstII was also cloned as a translation fusion to the Neisserial polysialyltransferase SiaD to make a self-priming polysialyltransferase as described by Willis et al (Willis L M, Gilbert M, Karwaski M-F, Blanchard M-C, Wakarchuk W W: Characterization of the α-2,8-polysialyltransferase from Neisseria meningitidis with synthetic acceptors, and the development of a self-priming polysialyltransferase fusion enzyme. Glycobiology 2008, 18(2):177-186). Two versions were cloned using homologous recombination in Saccharomyces cerevisiae resulting in plasmids pCstII-SiaD-Y and pCstII153S-SiaD-Y, the latter of which includes a mutation of isoleusine 53 to cysteine which is reported to improve the α2,8 sialyltransferase activity.

An acceptor glycoprotein was first prepared by addition of the T antigen-containing glycan to the MBP4XGT protein. Plasmids pMBP4XGT-Y and pDisJ-07 were used to transform strain MC4100ΔwaaL. The resulting strain was used to inoculate a 1L culture containing LB, ampicillin (100 ug/μl), and chloramphenicol (25 ug/μl). The culture is incubated at 30° C. until the optical density reaches 1.5 and then both glycan and glycoprotein production are induced with 0.2% arabinose and 1 mM IPTG respectively. The pellet is harvested after 16 hours and the his-tagged protein is purified by nickel affinity chromatography. Eluted protein is buffer exchanged into ex vivo sialylation buffer containing 50 mM Tris 7.5, 10 mM MgCl₂ and concentrated.

To prepare the polysialyltransferase lysates, strains MC4100ΔwaaL contining plasmid pTRCY, pCstII-SiaD-Y, or pCstII153S-SiaD-Y were grown in 50 mL cultures contining LB and ampicillin. When the optical density reached 1-5-1.9, protein expression is induced with the addition of IPTG to a final concentration of 1 mM and induction is carried out at 20° C. for approximately 16 hours. Pellets are harvested and resupended in ex vivo sialylation buffer. Following cell lysis, the material is centrifuges at 1000×g for 11 minutes and the supernatant is retained.

For the ex vivo reaction, 20 μl of the MBP glycoprotein is combined with 30 μl of the polysialylation or control lysate and CMP-NeuNAc. Reactions are incubated at 37° C. for 45 minutes prior to analysis by SDS-PAGE and Western blot (FIG. 10). Incubation with anti PSA antiserum (FIG. 10, top panel) resulted in appearance of high molecular weight material in the presene of both CMP-NeuNAc and lysate containing pCstII153S-SiaD-Y consistent with the formation of a PSA glycan. It appeared that there was a reduced amount of reactive material generated with the lysate containing the pCstII-SiaD-Y plasmid and none detected with the vector control. The presence of the MBP4XGT protein was confirmed with an anti-Histidine Western blot (FIG. 10, lower panel).

### Example 12

### Cloning and Expression of Genetic Machinery for PSA Capsular Synthesis in E. coli

The N-glycosylation pathway of bacteria has significant similarities to the polymerase-dependent pathway for the synthesis of O-antigen in many Gram-negative bacteria [55]. O-antigen is the outer component of lipopolysaccharide (LPS) and the major contributor to the antigenic variability of the bacterial cell surface [52]. O-antigen biosynthesis starts with the transfer of a sugar phosphate from a UDP-donor to an undecaprenyl phosphate (UndP) carrier. Different glycosyltransferases sequentially add the remaining monosaccharides from nucleotide-activated donors to complete the lipid-linked O-antigen subunit that is then translocated to the periplasmic side of the inner membrane by the Wzx flippase [56]. In the periplasm, Wzy catalyzes the polymerization of the O-antigen subunits. The polymerized O-antigen is transferred to the lipid A core to form LPS in a step involving the O-antigen ligase WaaL [52] and subsequently transported to the outer membrane. O-antigen exhibits a strain-specific size distribution pattern, which is mediated by the Wzz protein [57]. In pgl+E. coli cells, protein N-glycosylation and O-antigen biosynthesis converge at the step in which PglB, the key enzyme of the C. jejuni N-glycosylation system, transfers O polysaccharide from the UndP lipid carrier to an acceptor protein. Inactivation of the O-antigen ligase (WaaL) in pgl+ E. coli cells results in the accumulation of UndP-linked polysaccharide and PglB-mediated transfer of O-antigen to the protein acceptor [31].

E. coli K1 are encapsulated with the α(2-8)-polysialic acid NeuNAc(α2-8), common to several bacterial pathogens. The pathway for PSA capsule synthesis involves: (i) formation of the precursor, CMP-NeuNAc, (ii) polymerization of sialic acid, and (iii) export of the polymer to the cell surface. The gene cluster encoding the pathway for synthesis of this polymer is organized into three regions: (i) kpsSCUDEF, (ii) neuDBACES, and (iii) kpsMT. Similar to O-antigens which are displayed as part of LPS, PSA K-antigens are Group 2 capsules displayed on the cell surface as capsular polysaccharides [59]. Thus, based on the observation that prevention of O-antigen transfer to the lipid A-core created a pool of substrates for glycosylation[31], it is contemplated that similarly disrupting export of PSA to the cell surface will result in a pool of K-antigen substrates for N-glycosylation. Hence, one strategy is to clone the genes responsible for (i) formation of the precursor, CMP-NeuNAc and (ii) polymerization of sialic acid; but exclude the genes responsible for (iii) export of the polymer to the cell surface.

For formation of the CMP-NeuNAc precursor from UDP-GlcNAc, genes encoding, NeuB (synthase), NeuC (epimerase), and NeuA (synthase) are cloned [60]. For polymerization of sialic acid, NeuS is the sole polysialyltransferase, yet it cannot synthesize PSA de novo without other products of the gene cluster—even in the presence of CMP-NeuNAc substrate [20]. In fact, it was recently shown that minimally NeuES and KpsCS are required to synthesize PSA at high levels from CMP-NeuNAc substrate in isolated membranes [61]. Thus, a minimal PSA synthesis module is cloned that includes the genes encoding NeuS, NeuE, KpsC, KpsS [61]. All other genes, notably those encoding the ABC transporter (KpsM, KpsT) and those responsible for mediating translocation to the cell surface (KpsE, KpsD), are excluded [59]. The targeted genes are cloned into the pACYC184 vector, as used previously for the pgl operon [35]. Specifically, E. coli K1 genomic DNA is isolated and neuDBACES and kpsSC are amplified using oligonucleotide primers and standard PCR. The resulting PCR-amplified DNA is cloned into pACYC 184 using standard molecular cloning techniques. The resulting plasmid is sequenced and transformed into E. coli. An existing plasmid (pBA6HP) for bicistronic expression of the C. jejuni OST PglB and the acceptor glycoprotein AcrA is co-transformed into these cells. Following expression and purification, AcrA is subjected to SDS-PAGE and Western blot analysis with primary antibodies specific for AcrA or specific for PSA (Millipore). Since AcrA has served as a model glycoprotein in a number of bacterial hosts [35,58], conjugation of AcrA is the first benchmark of success in this proposal. Successful PSA-conjugation to AcrA will indicate likelihood of successful production of PSA-conjugated insulin.

Synthesis of PSA mediated by the cloned genes is confirmed by subjecting cell extracts to the SIALICQ Sialic Acid Quantitation Kit (Sigma) according to manufacturer's instructions. The kit uses α(2-3,6,8,9) neuraminidase to cleave all sialic acid linkages, including α(2-8), α(2-9), and branched linkages, for the most accurate determination of extracellular polysialic acid content. This analysis can quantify the amount of N-acetylneuraminic acid produced by cells either free, or in glycoproteins, cell surface glycoproteins, polysialic acids and capsular polysaccharides. Since laboratory strains of E. coli lack genes for sialic acid synthesis of any kind, detection of background sialic acid will not be an issue. Thus, detection of sialic acid is a performance benchmark indicative of effective cloning of the genes necessary for PSA synthesis.

Cloning Genes for PSA Synthesis

The neuDBACES and kpsSC genes were cloned from E. coli via homologous recombination in the yeast Saccharomyces cerevisiae as previously described (Shanks et al., 2006) to generate a single plasmid that expresses neuDBACESkpsSC as a transcriptional unit. In this system, regions of homologous DNA are used to target recombination events: in this case, between a yeast/bacterial shuttle vector and PCR products. Briefly, the neuDBACES and kpsSC genes were amplified as separate units from genomic DNA. The primers used to generate these products were designed to incorporate approximately 40 terminal nucleotides that share homology with the vector, and 40 nucleotides of homology between the neu and kps amplicons. The vector and PCR products were simultaneously transformed into S. cerevisiae and a single plasmid was synthesized via recombination at the sites of homology.

### Example 13

### In Vivo Synthesis of Proteins Carrying an N-Glycan Terminating in the Human Blood Group O Glycan (H-Antigen)

The human blood group O determinant or H-antigen consists of a fucosylated glycan that resembles the human T antigen. The type III H-antigen structure consists of Fucose α1,2 Galactose β1,3 GalNAc α-. To synthesize a glycan in E. coli terminating in the human H-antigen structure, the plasmid described above expressing genes required to synthesize the T-antigen glycan (pDisJ-07) was modified to include a gene encoding a fucosyltransferase. The resulting plasmid, pDisJK-07, contains a synthetic operon under control of the pBAD promoter with genes in the following order: galE, pglB, pglA, wbnJ, wbnK.

Fucosyltransferase WbnK from E. coli 086 was selected because it fucosylates a glycan with similar structure in its native context. A PCR product containing the wbnJ and wbnK genes was generated using a synthetic template from Genewiz. The PCR product was combined with linear pDis-07 plasmid using homologous recombination in yeast to generate plasmid pDisJK-07.

For use in expressing fucosylated blood group H-antigen, the E. coli strain LPS1 (Yavuz E, Maffioli C, Ilg K, Aebi M, Priem B: Glycomimicry: display of fucosylation on the lipo-oligosaccharide of recombinant Escherichia coli K12. Glycoconjugate journal 2011, 28(1):39-47) was used to promote accumulation of GDP-fucose (GDP-Fuc). E. coli encodes a native pathway for synthesis of GDP-Fuc however this sugar nucleotide is then normally incorporated into the fucose-containing exopolysaccharide colanic acid. To prevent usage of GDP-Fuc in this competing pathway a mutation is present in the gene wcaJ (ECK2041) encoding a putative UDP-glucose lipid carrier transferase. To further promote glycosylation in this strain, a mutation in the waaL gene was introduced. The waaL (rfaL) gene has been previously mutated as part of the Keio collection and the resulting strain rfaL734(del)::kan (JW3597-1) (Baba et al.) was obtained from the Yale Coli Genetic Stock Center (CGSC). P1 vir phage was used to transduce the waaL mutation into th LPS 1 recipient to make strain LPS 1 ΔwaaL::kan.

To confirm the glycan structure produced by the glycosyltransferases encoded by pDisJK-07, the plasmid was used to transform strain LPS1ΔwaaL::kan for analysis of the lipid-released oligosaccharides. A 250 mL culture of the resulting strain was grown at 30° C. and induced when the optical density reached an ABS₆₀₀ around˜2.0. Cells were harvested after ˜20 hours for isolation of lipid-linked oligosaccharides by the method of Gao and Lehrman. Briefly, pellet was resuspended in 10 mL methanol and lysed by sonication. Material was dried at 60° C. and subsequently resuspended in 1 mL 2:1 chloroform:methanol (v/v, CM) via sonication and material was washed two times in CM. The pellet was then washed in water then lipids were extracted with 10:10:3 chloroform:methanol: water (v/v/v, CMW) followed by methanol. The CMW and methanol extracts were combined and loaded onto a DEAE cellulose column. CMW was used to wash the column and lipid-linked oligosaccharides were eluted with 300 mM NH₄OAc in CMW. The lipid-linked oligosaccharides were extracted with chloroform and dried.

To release the glycans from the lipids, the material was resuspended in 1.5 mL 0.1N HCl in 1:lisopropanol: water (v/v). The solution was heated at 50° C. for 2 hours and then dried at 75° C. Residue was suspended in water saturated butanol and the aqueous phase containing the glycans was dried, resuspended in water, and purified with AG50W-H8(hydrogen atom) cation exchange resin followed by Agl-X8 (formate form) anion exchange resin.

Purified oligosaccharides solubilized in water were subjected to incubation with α1,2 fucosidase (NEB) treatment) or a buffer only control and analyzed on an AB SCIEX TOF/TOF mass spectrometer using dihydroxybenzoic acid (DHB) as the matrix (FIG. 11a). In the buffer control (top panel), two major peaks present (m/z 755) and (m/z 609) are consistent with the expected (m/z) of the fucosylated product (Fuc Hex HexNAc₂) and the T antigen glycan (Hex Hex NAc₂) respectively. Following fucosidase treatment (bottom panel), the peak at (m/z 755) is greatly reduced while the peak at (m/z 609) is relatively larger. The difference between these peaks (146) is consistant with the size of a fucose residue.

### Example 14

### Improving Relative Fucosylation Through Expression of GDP-Fucose Biosynthetic Genes

In order to improve conversion from the T antigen glycan to the fucosylated product, a system was devised in order to allow for expression of additional copies of the biosynthetic machinery for GDP-Fucose, UDP-Gal, and UDP-GalNAc. To accomplish this, the following genes were cloned as a synthetic operon under control of the pBAD promoter: galE (C. jejuni), galE, gmd, fcl, gmm, cpsB, cpsG (E. coli) to make plasmid pGNF-70.

Strain LPS 1 ΔwaaL::kan was transformed with plasmids pJK-07 and pGNF-70. The resulting strain was cultured in 250 mL LB medium under ampicillin and chloramphenicol selection and expression of both plasmids was induced at an optical density of approximately 2.0 and induction continued at 30° C. for approximately 16 hours. Pellets were harvested and LLOs were purified as previously described by the method of Gao and Lehrman.

Purified oligosaccharides were analyzed by Mass Spectrometry as described above (FIG. 11b). The major peak identified following this treatment (m/z 755) is consistant with the desired fucosylated glycan (dHex Hex HexNAc₂). An addition peak is present at (m/z 609) which is consistant with the glycan (Hex HexNAc₂).

### Example 15

### Generating a Fucosylated Glycoprotein In Vivo in E. coli

Following analysis of the fucosylated glycan, it is necessary to confirm that the glycan is amenable to use in the glycosylation reaction. The TNFa Fab was selected as an initial target for glycosylation. A codon optimized version of the Fab was obtained from DNA 2.0 and cloned into pTRCY using homologous recombination in S. cerevisiae to append a 4×GlycTag and hexahistidine tag (SEQ ID NO: 36) to the heavy chain. The resulting plasmid is designated pTnfαFab4X-Y.

pTnfαFab4X-Y was used to transform strain LPS 1 Atrain LPS bearing glycosylation plasmid pJK-07 or pMW07 and the resulting strains were used to inoculate a 50 mL culture of LB and grown under selection of ampicillin and chloramphenicol. At an optical density of ABS600 of 1.5, expression of both plasmids was induced with the addition of 0.2% arabinose and 1 mM IPTG and cultures were maintained at 30° C. for approximately 16 hours. Protein was purified using nickel affinity chromatography and protein was subjected to SDS PAGE followed by Western blot with anti Histidine antibody. A mobility shift was apparent for the Fab heavy chain grown in the presence of glycosylation plasmid pJK-07 but not vector pMW07 consistent with glycosylation (FIG. 12).

### Example 16

### Generating a Fucosylated Glycopeptide In Vivo in E. coli Modified with the Blood Group H-Antigen

Previous studies indicated that the ratio of the fucosylated peak to afucosylated product as determined by Mass spectrometry is improved through expression of additional copies of the GDP-Fucose biosynthetic pathway. A plasmid pMG1X-Y encoding the glycosylation acceptor peptide is modified using yeast homologous recombination to also include the following genes: galE (C. jejuni), galE (E. coli), gmd, fcl, gmm, cpsB, and cpsG to make plasmid pMG1X-GNF-Y. A similar plasmid was cloned in the same manner with the following genes in addition to the glucagon construct: wbnK, galE (E. coli), gmd, fcl, gmm, cpsB, and cpsG termed pMG1X-KGF-Y.

In preparation for glycosylation, strain LPS 1 is transformed with plasmid pDisJ-07. To this, plasmids encoding the glycosylation acceptor protein (pMG1X-Y) or the acceptor protein with the GDP-Fucose biosynthetic machinery were added (pMG1X-GNF-Y, pMG1X-KGF-Y). Resulting strains were grown at 30° C. in 50 mL cultures in LB medium with ampicillin and chloramphenicol. Both plasmids were induced with the addition of arabinose and 1 mM IPTG when the culture reached an approximate optical density of ABS600 1.5. After 16 hours, pellets were harvested and proteins purified by nickel affinity chromatography. Eluate was buffer exchanged into 50 mM Tris, 100 mM NaCl and 30 μl of the concentrated protein was treated with TEV protease for 3 hours to release the glycopeptide.

Glycopeptide was analyzed on an AB SCIEX TOF/TOF mass spectrometer using dihydroxybenzoic acid (DHB) as the matrix (FIG. 13). Peaks consistant with the expected sizes of the fucosylated glycopeptide (dHex Hex HexNAc₂, m/z 6103) and galactosylated glycopeptide (Hex HexNAc₂, m/z 5957) are present in glycopeptide prepared from the strain with plasmid pMG1X (left). Side product is marked with an asterick. Glycopeptide from the strain harboring pMG1X GNF-Y exhibited one major peak consistant with the expected m/z of the H-antigen glycopeptide (dHex Hex HExNAc₂, m/z 6105). An additional smaller peak at (m/z 5960) is also present likely representing remaining unfucoyslated glycopeptide containing the T antigen glycan (Hex HexNAc₂).

Glycopeptide prepared from strain LPS1 pJK-07 pMG1X KGF-Y was divided and subjected to treatment with α1,2 fucosidase (NEB) or a buffer control for 8 hours at 37 degrees prior to analysis on an AB SCIEX TOF/TOF mass spectrometer using DHB as the matrix (FIG. 14). The major peak present in the buffer-only sample (m/z 6107) is consistent with the expected size of the H-antigen containing glycan (dHex Hex HexNAc₂). The sample treated treated with fucosidase has a major peak at (m/z 5963) consistent with the expected size of the gal terminal T antigen glycan (Hex HexNAc₂).

INFORMAL SEQUENCE LISTINGS

pMW07: vector
7610 bp ds-DNA
Sequence ID No 1

| | |
|---|---|
| 1 | gatttatctt cgtttcctgc aggtttttgt tctgtgcagt tgggttaaga atactgggca |
| 61 | atttcatgtt tcttcaacac tacatatgcg tatatatacc aatctaagtc tgtgctcctt |
| 121 | ccttcgttct tccttctgtt cggagattac cgaatcaaaa aaatttcaaa gaaaccgaaa |
| 181 | tcaaaaaaaa gaataaaaaa aaaatgatga attgaattga aaagctgtgg tatggtgcac |
| 241 | tctcagtaca atctgctctg atgccgcata gttaagccag ccccgacacc cgccaacacc |
| 301 | cgctgacgcg ccctgacggg cttgtctgct cccggcatcc gcttacagac aagctgtgac |
| 361 | cgtctccggg agctgcatgt gtcagaggtt ttcaccgtca tcaccgaaac gcgcgagacg |
| 421 | aaagggcctc gtgatacgcc tatttttata ggttaatgtc atgataataa tggtttctta |
| 481 | ggacggatcg cttgcctgta acttacacgc gcctcgtatc ttttaatgat ggaataattt |
| 541 | gggaatttac tctgtgttta tttattttta tgttttgtat ttggatttta gaaagtaaat |
| 601 | aaagaaggta gaagagttac ggaatgaaga aaaaaaaata aacaaaggtt taaaaaattt |
| 661 | caacaaaaag cgtactttac atatatattt attagacaag aaaagcagat taaatagata |
| 721 | tacattcgat taacgataag taaaatgtaa aatcacagga ttttcgtgtg tggtcttcta |
| 781 | cacagacaag atgaaacaat tcggcattaa tacctgagag caggaagagc aagataaaag |
| 841 | gtagtatttg ttggcgatcc ccctagagtc ttttacatct tcggaaaaca aaaactattt |
| 901 | tttctttaat ttcttttttt actttctatt tttaatttat atatttatat taaaaaattt |
| 961 | aaattataat tatttttata gcacgtgatg aaaaggaccc aggtggcact tttcggggaa |
| 1021 | atgtgcgcgg aacccctatt tgtttatttt tctaaataca ttcaaatatg tatccgctca |
| 1081 | tgagacaata accctgataa atgcttcaat aatattgaaa aaggaagagt atgagtattc |
| 1141 | aacatttccg tgtcgccctt attccctttt ttgcggcatt ttgccttcct gtttttgctc |
| 1201 | acccagaaac gctggtgaaa gtaaaagatg ctgaagatca gtttaagggc accaataact |
| 1261 | gccttaaaaa aattacgccc cgccctgcca ctcatcgcag tactgttgta attcattaag |
| 1321 | cattctgccg acatggaagc catcacagac ggcatgatga acctgaatcg ccagcggcat |
| 1381 | cagcaccttg tcgccttgcg tataatattt gcccatggtg aaaacggggg cgaagaagtt |
| 1441 | gtccatattg gccacgttta aatcaaaact ggtgaaactc acccagggat tggctgagac |
| 1501 | gaaaaacata ttctcaataa accctttagg gaaataggcc aggttttcac cgtaacacgc |
| 1561 | cacatcttgc gaatatatgt gtagaaactg ccggaaatcg tcgtggtatt cactccagag |
| 1621 | cgatgaaaac gtttcagttt gctcatggaa aacggtgtaa caagggtgaa cactatccca |
| 1681 | tatcaccagc tcaccgtctt tcattgccat acggaattcc ggatgagcat tcatcaggcg |
| 1741 | ggcaagaatg tgaataaagg ccggataaaa cttgtgctta tttttcttta cggtctttaa |
| 1801 | aaaggccgta atatccagct gaacggtctg gttataggta cattgagcaa ctgactgaaa |
| 1861 | tgcctcaaaa tgttctttac gatgccattg ggatatatca acggtggtat atccagtgat |
| 1921 | ttttttctcc attttagctt ccttagctcc tgaaaatctc gataactcaa aaaatacgcc |
| 1981 | cggtagtgat cttatttcat tatggtgaaa gttggaacct cttacgtgcc gatcaacgtc |
| 2041 | tcattttcgc caaaagttgg cccagggctt cccggtatca acagggacac caggatttat |
| 2101 | ttattctgcg aagtgatctt ccgtcacagg tatttattcg gcgcaaagtg cgtcgggtga |
| 2161 | tgctgccaac ttactgattt agtgtatgat ggtgtttttg aggtgctcca gtggcttctg |
| 2221 | tttctatcag ctgtccctcc tgttcagcta ctgacggggt ggtgcgtaac ggcaaaagca |
| 2281 | ccgccggaca tcagcgctag cggagtgtat actggcttac tatgttggca ctgatgaggg |
| 2341 | tgtcagtgaa gtgcttcatg tggcaggaga aaaaaggctg caccggtgcg tcagcagaat |
| 2401 | atgtgataca ggatatattc cgcttcctcg ctcactgact cgctacgctc ggtcgttcga |
| 2461 | ctgcggcgag cggaaatggc ttacgaacgg ggcggagatt tcctggaaga tgccaggaag |
| 2521 | atacttaaca gggaagtgag agggccgcgg caaagccgtt tttccatagg ctccgccccc |
| 2581 | ctgacaagca tcacgaaatc tgacgctcaa atcagtggtg gcgaaacccg acaggactat |
| 2641 | aaagatacca ggcgtttccc cctggcggct ccctcgtgcg ctctcctgtt cctgcctttc |
| 2701 | ggtttaccgg tgtcattccg ctgttatggc cgcgtttgtc tcattccacg cctgacactc |
| 2761 | agttccgggt aggcagttcg ctccaagctg gactgtatgc acgaaccccc cgttcagtcc |
| 2821 | gaccgctgcg ccttatccgg taactatcgt cttgagtcca acccggaaag acatgcaaaa |
| 2881 | gcaccactgg cagcagccac tggtaattga tttagaggag ttagtcttga agtcatgcgc |
| 2941 | cggttaaggc taaactgaaa ggacaagttt tggtgactgc gctcctccaa gccagttacc |
| 3001 | tcggttcaaa gagttggtag ctcagagaac cttcgaaaaa ccgccctgca aggcggtttt |
| 3061 | ttcgttttca gagcaagaga ttacgcgcag accaaaacga tctcaagaag atcatcttat |
| 3121 | taatcagata aaatatttgc tcatgagccc gaagtggcga gcccgatctt ccccatcggt |
| 3181 | gatgtcggcg atataggcgc cagcaaccgc acctgtggcg ccggtgatgc cggccacgat |
| 3241 | gcgtccggcg tagaggatct gctcatgttt gacagcttat catcgatgca taatgtgcct |
| 3301 | gtcaaatgga cgaagcaggg attctgcaaa ccctatgcta ctccgtcaag ccgtcaattg |
| 3361 | tctgattcgt taccaattat gacaacttga cggctacatc attcactttt tcttcacaac |
| 3421 | cggcacggaa ctcgctcggg ctggccccgg tgcatttttt aaatacccgc gagaaataga |
| 3481 | gttgatcgtc aaaaccaaca ttgcgaccga cggtggcgat aggcatccgg gtggtgctca |
| 3541 | aaagcagctt cgcctggctg atacgttggt cctcgcgcca gcttaagacg ctaatcccta |
| 3601 | actgctggcg gaaaagatgt gacagacgcg acggcgacaa gcaaacatgc tgtgcgacgc |
| 3661 | tggcgatatc aaaattgctg tctgccaggt gatcgctgat gtactgacaa gcctcgcgta |
| 3721 | cccgattatc catcggtgga tggagcgact cgttaatcgc ttccatgcgc cgcagtaaca |
| 3781 | attgctcaag cagatttatc gccagcagct ccgaatagcg cccttcccct tgcccggcgt |
| 3841 | taatgatttg cccaaacagg tcgctgaaat gcggctggtg cgcttcatcc gggcgaaaga |
| 3901 | accccgtatt ggcaaatatt gacggccagt taagccattc atgccagtag gcgcgcggac |
| 3961 | gaaagtaaac ccactggtga taccattcgc gagcctccgg atgacgaccg tagtgatgaa |
| 4021 | tctctcctgg cgggaacagc aaaatatcac ccggtcggca aacaaattct cgtccctgat |
| 4081 | ttttcaccac cccctgaccg cgaatggtga gattgagaat ataacctttc attcccagcg |
| 4141 | gtcggtcgat aaaaaaatcg agataaccgt tggcctcaat cggcgttaaa cccgccacca |
| 4201 | gatgggcatt aaacgagtat cccggcagca ggggatcatt ttgcgcttca gccatacttt |
| 4261 | tcatactccc gccattcaga gaagaaacca attgtccata ttgcatcaga cattgccgtc |
| 4321 | actgcgtctt ttactggctc ttctcgctaa ccaaaccggt aaccccgctt attaaaagca |
| 4381 | ttctgtaaca aagcgggacc aaagccatga caaaaacgcg taacaaaagt gtctataatc |
| 4441 | acggcagaaa agtccacatt gattatttgc acggcgtcac actttgctat gccatagcat |
| 4501 | ttttatccat aagattagcg gatcctacct gacgcttttt atcgcaactc tctactgttt |
| 4561 | ctccataccc gtttttttgg gctagcgaat tcgagctcgg tacccgggga tcctctagag |
| 4621 | tcgacctgca ggcatgcaag cttggctgtt ttggcggatg agagaagatt ttcagcctga |
| 4681 | tacagattaa atcagaacgc agaagcggtc tgataaaaca gaatttgcct ggcggcagta |
| 4741 | gcgcggtggt cccacctgac cccatgccga actcagaagt gaaacgccgt agcgccgatg |
| 4801 | gtagtgtggg gtctccccat gcgagagtag ggaactgcca ggcatcaaat aaaacgaaag |
| 4861 | gctcagtcga aagactgggc ctttcgtttt atctgttgtt tgtcggtgaa cgctctcctg |
| 4921 | agtaggacaa atccgccggg agcggatttg aacgttgcga agcaacggcc cggagggtgg |
| 4981 | cgggcaggac gcccgccata aactgccagg catccttgca gcacatcccc ctttcgccag |
| 5041 | ctggcgtaat agcgaagagg cccgcaccga tcgcccttcc caacagttgc gcagcctgaa |
| 5101 | aggcaggccg ggccgtggtg gccacggcct ctaggccaga tccagcggca tctgggttag |
| 5161 | tcgagcgcgg gccgcttccc atgtctcacc agggcgagcc tgtttcgcga tctcagcatc |
| 5221 | tgaaatcttc ccggccttgc gcttcgctgg ggccttaccc accgccttgg cgggcttctt |
| 5281 | cggtccaaaa ctgaacaaca gatgtgtgac cttgcgcccg gtctttcgct gcgcccactc |
| 5341 | cacctgtagc gggctgtgct cgttgatctg cgtcacggct ggatcaagca ctcgcaactt |
| 5401 | gaagtccttg atcgagggat accggccttc cagttgaaac cactttcgca gctggtcaat |
| 5461 | ttctatttcg cgctggccga tgctgtccca ttgcatgagc agctcgtaaa gcctgatcgc |
| 5521 | gtgggtgctg tccatcttgg ccacgtcagc caaggcgtat ttggtgaact gtttggtgag |
| 5581 | ttccgtcagg tacggcagca tgtctttggt gaacctgagt tctacacggc cctcaccctc |
| 5641 | ccggtagatg attgtttgca cccagccggt aatcatcaca ctcggtcttt tccccttgcc |
| 5701 | attgggctct tgggttaacc ggacttcccg ccgtttcagg cgcagggccg cttctttgag |
| 5761 | ctggttgtag gaagattcga tagggacacc cgccatcgtc gctatgtcct ccgccgtcac |
| 5821 | tgaatacatc acttcatcgg tgacaggctc gctcctcttc acctggctaa tacaggccag |
| 5881 | aacgatccgc tgttcctgaa cactgaggcg atacgcggcc tcgaccaggg cattgctttt |
| 5941 | gtaaaccatt gggggtgagg ccacgttcga cattccttgt gtataagggg acactgtatc |
| 6001 | tgcgtcccac aatacaacaa atccgtccct ttacaacaac aaatccgtcc cttcttaaca |
| 6061 | acaaatccgt cccttaatgg caacaaatcc gtcccttttt aaactctaca ggccacggat |
| 6121 | tacgtggcct gtagacgtcc taaaaggttt aaaagggaaa aggaagaaaa gggtggaaac |
| 6181 | gcaaaaaacg caccactacg tggccccgtt ggggccgcat ttgtgcccct gaaggggcgg |
| 6241 | gggaggcgtc tgggcaatcc ccgttttacc agtcccctat cgccgcctga gagggcgcag |
| 6301 | gaagcgagta atcagggtat cgaggcggat tcacccttgg cgtccaacca gcggcaccag |
| 6361 | cggctcgaca acccttaata taacttcgta taatgtatgc tatacgaagt tattaggtct |
| 6421 | agagatctgt ttagcttgcc tcgtccccgc cgggtcagcc ggcggttaag gtatactttc |
| 6481 | cgctgcataa ccctgcttcg gggtcattat agcgattttt tcggtatatc catccttttt |
| 6541 | cgcacgatat acaggatttt gccaaagggt tcgtgtagac tttccttggt gtatccaacg |
| 6601 | gcgtcagccg ggcaggatag gtgaagtagg cccacccgcg agcgggtgtt ccttcttcac |
| 6661 | tgtcccttat tcgcacctgg cggtgctcaa cgggaatcct gctctgcgag gctggccgat |
| 6721 | aagctccacg tgaataactg atataattaa attgaagctc taatttgtga gtttagtata |
| 6781 | catgcattta cttataatac agttttttag ttttgctggc cgcatcttct caaatatgct |
| 6841 | tcccagcctg cttttctgta acgttcaccc tctaccttag catcccttcc ctttgcaaat |
| 6901 | agtcctcttc caacaataat aatgtcagat cctgtagaga ccacatcatc cacggttcta |
| 6961 | tactgttgac ccaatgcgtc tcccttgtca tctaaaccca caccgggtgt cataatcaac |
| 7021 | caatcgtaac cttcatctct tccacccatg tctctttgag caataaagcc gataacaaaa |
| 7081 | tctttgtcgc tcttcgcaat gtcaacagta cccttagtat attctccagt agatagggag |
| 7141 | cccttgcatg acaattctgc taacatcaaa aggcctctag gttcctttgt tacttcttct |
| 7201 | gccgcctgct tcaaaccgct aacaatacct gggcccacca caccgtgtgc attcgtaatg |
| 7261 | tctgcccatt ctgctattct gtatacaccc gcagagtact gcaatttgac tgtattacca |
| 7321 | atgtcagcaa attttctgtc ttcgaagagt aaaaaattgt acttggcgga taatgccttt |
| 7381 | agcggcttaa ctgtgccctc catggaaaaa tcagtcaaga tatccacatg tgtttttagt |
| 7441 | aaacaaattt tgggacctaa tgcttcaact aactccagta attccttggt ggtacgaaca |
| 7501 | tccaatgaag cacacaagtt tgtttgcttt tcgtgcatga tattaaatag cttggcagca |
| 7561 | acaggactag gatgagtagc agcacgttcc ttatatgtag ctttcgacat |

//
galE: epimerase, C. jejuni
EC 5.1.3.2
987 bp ds-DNA
SEQ ID NO 2

| | |
|---|---|
| 1 | atgaaaattc ttattagcgg tggtgcaggt tatataggtt ctcatacttt aagacaattt |
| 61 | ttaaaaacag atcatgaaat ttgtgtttta gataatcttt ctaagggttc taaaatcgca |
| 121 | atagaagatt tgcaaaaaat aagaactttt aaattttttg aacaagattt aagtgatttt |
| 181 | caaggcgtaa aagcattgtt tgagagagaa aaatttgacg ctattgtgca ttttgcagcg |
| 241 | agcattgaag tttttgaaag tatgcaaaac cctttaaagt attatatgaa taacactgtt |
| 301 | aatacgacaa atctcatcga aacttgtttg caaactggag tgaataaatt tatattttct |
| 361 | tcaacggcag ccacttatgg cgaaccacaa actcccgttg tgagcgaaac aagtccttta |
| 421 | gcacctatta atccttatgg gcgtagtaag cttatgagcg aagaggtttt gcgtgatgca |
| 481 | agtatggcaa atcctgaatt taagcattgt attttaagat attttaatgt tgcaggtgct |
| 541 | tgcatggatt atactttagg acaacgctat ccaaaagcga ctttgcttat aaaagttgca |
| 601 | gctgaatgtg ccgcaggaaa acgtaataaa cttttcatat ttggcgatga ttatgataca |
| 661 | aaagatggca cttgcataag agattttatc catgtggatg atatttcaag tgcgcattta |
| 721 | tcggctttgg attatttaaa agagaatgaa agcaatgttt ttaatgtagg ttatggacat |
| 781 | ggttttagcg taaaagaagt gattgaagcg atgaaaaaag ttagcggagt ggattttaaa |
| 841 | gtagaacttg ccccacgccg tgcgggtgat cctagtgtat tgatttctga tgcaagtaaa |
| 901 | atcagaaatc ttacttcttg gcagcctaaa tatgatgatt tagggcttat ttgtaaatct |
| 961 | gcttttgatt gggaaaaaca gtgctaa |

//
pglB: OST, C. jejuni
EC 2.4.1.119
2142 bp ds-DNA
SEQ ID NO 3

| | |
|---|---|
| 1 | atgttgaaaa aagagtattt aaaaaaccct tatttagttt tgtttgcgat gattatatta |
| 61 | gcttatgttt ttagtgtatt ttgcaggttt tattgggttt ggtgggcaag tgagtttaat |
| 121 | gagtattttt tcaataatca gttaatgatc atttcaaatg atggctatgc ttttgctgag |
| 181 | ggcgcaagag atatgatagc aggttttcat cagcctaatg atttgagtta ttatggatct |
| 241 | tctttatccg cgcttactta ttggctttat aaaatcacac ctttttcttt tgaaagtatc |
| 301 | attttatata tgagtacttt tttatcttct ttggtggtga ttcctactat tttgctagct |
| 361 | aacgaataca aacgtccttt aatgggcttt gtagctgctc ttttagcaag tatagcaaac |
| 421 | agttattata atcgcactat gagtgggtat tatgatacgg atatgctggt aattgttttg |
| 481 | cctatgttta ttttattttt tatggtaaga atgattttaa aaaaagactt tttttcattg |
| 541 | attgccttgc cgttatttat aggaatttat ctttggtggt atccttcaag ttatacttta |
| 601 | aatgtagctt taattggact ttttttaatt tatacactta tttttcatag aaaagaaaag |
| 661 | attttttata tagctgtgat tttgtcttct cttactcttt caaatatagc atggttttat |
| 721 | caaagtgcca ttatagtaat actttttgct ttattcgcct tagagcaaaa acgcttaaat |
| 781 | tttatgatta taggaatttt aggtagtgca actttgatat ttttgatttt aagtggtggg |
| 841 | gttgatccta tactttatca gcttaaattt tatattttta gaagtgatga aagtgcgaat |
| 901 | ttaacgcagg gctttatgta ttttaatgtc aatcaaacca tacaagaagt tgaaaatgta |
| 961 | gatcttagcg aatttatgcg aagaattagt ggtagtgaaa ttgttttttt gttttctttg |
| 1021 | tttggttttg tatggctttt gagaaaacat aaaagtatga ttatggcttt acctatattg |
| 1081 | gtgcttgggt ttttagcctt aaaagggggg cttagattta ccatttattc tgtacctgta |
| 1141 | atggccttag gatttggttt tttattgagc gagtttaagg ctataatggt taaaaaatat |
| 1201 | agccaattaa cttcaaatgt ttgtattgtt tttgcaacta ttttgacttt agctccagta |
| 1261 | tttatccata tttacaacta taaagcgcca acagtttttt ctcaaaatga agcatcatta |
| 1321 | ttaaatcaat taaaaaatat agccaataga gaagattatg tggtaacttg gtgggattat |
| 1381 | ggttatcctg tgcgttatta tagcgatgtg aaaactttag tagatggtgg aaagcattta |
| 1441 | ggtaaggata attttttccc ttcttttgct ttaagcaaag atgaacaagc tgcagctaat |
| 1501 | atggcaagac ttagtgtaga atatacagaa aaaagctttt atgctccgca aaatgatatt |
| 1561 | ttaaaaacag acattttgca agccatgatg aaagattata atcaaagcaa tgtggatttg |
| 1621 | tttctagctt cattatcaaa acctgatttt aaaatcgata cgccaaaaac tcgtgatatt |
| 1681 | tatctttata tgcccgctag aatgtctttg attttttcta cggtggctag tttttctttt |
| 1741 | attaatttag atacaggagt tttggataaa ccttttacct ttagcacagc ttatccactt |
| 1801 | gatgttaaaa atggagaaat ttatcttagc aacggagtgg ttttaagcga tgattttaga |
| 1861 | agttttaaaa taggtgataa tgtggtttct gtaaatagta tcgtagagat taattctatt |
| 1921 | aaacaaggtg aatacaaaat cactccaatt gatgataagg ctcagtttta tattttttat |
| 1981 | ttaaaggata gtgctattcc ttacgcacaa tttattttaa tggataaaac catgtttaat |
| 2041 | agtgcttatg tgcaaatgtt ttttttagga aattatgata agaatttatt tgacttggtg |
| 2101 | attaattcta gagatgctaa ggtttttaaa cttaaaattt aa |

//
pglA: α1,3-N-acetylgalactosamine transferase
EC 2.4.1.-
1131 bp ds-DNA
SEQ ID NO 4

| | |
|---|---|
| 1 | atgagaatag gatttttatc acatgcagga gcaagtattt atcattttag aatgcctatt |
| 61 | ataaaagcat taaaagatag aaaagatgaa gtttttgtta tagtgccgca agatgaatac |
| 121 | acgcaaaaac ttagagatct tggtttaaaa gtaattgttt atgagttttc aagagctagt |
| 181 | ttaaatcctt ttgtagtttt aaagaatttt ttttatcttg ctaaggtttt aaaaaattta |
| 241 | aatcttgatc ttattcaaag tgcggcacac aaaagcaata cctttggaat tttagcggca |
| 301 | aaatgggcaa aaattcctta tcgttttgct ttggtagaag gcttgggatc tttttatata |
| 361 | gatcaaggtt ttaaggcaaa tttagtacgt tttgttatta ataatcttta taaattaagt |
| 421 | tttaaatttg cacaccaatt tatttttgtc aatgaaagta atgccgagtt tatgcggaat |
| 481 | ttaggactta aggaaaataa aatttgtgtg ataaaatccg tagggatcaa tttaaaaaaa |
| 541 | ttttttccta tttatataga atcggaaaaa aaagagcttt tttggagaaa tttaaatata |
| 601 | gataaaaaac ctattgttct tatgatagca agagctttat ggcataaagg tgtaaaagaa |
| 661 | ttttatgaaa gtgctactat gctaaaagac aaagcaaatt ttgttttagt tggtggaaga |
| 721 | gatgaaaatc cttcttgtgc gagtttggag tttttaaact cgggtgtggt gcattatttg |
| 781 | ggtgctagaa gtgatatagt cgagcttttg caaaattgtg atatttttgt tttaccaagc |
| 841 | tataaagaag gctttcctgt aagtgttttg gaggcaaaag cttgtggcaa ggctatagtg |
| 901 | gtgagtgatt gtgaaggttg tgtagaggct atttctaatg cttatgatgg actttgggca |
| 961 | aaaacaaaaa atgctaagga tttaagcgaa aaaatttcac ttttattaga agatgaaaaa |
| 1021 | ttaagattaa atttagctaa aaatgctgcc caagatgctt tacaatacga tgaaaataat |
| 1081 | atcgcacagc gttatttaaa actttatgat agggtaatta agaatgtatg a |

//
wbnJ: β1,3 galactosyl transferase
EC 2.4.1-
765 bp ds-DNA
SEQ ID NO 5

| | |
|---|---|
| 1 | atgtcattga gaatattaga tatgatttca gtaataatgg ctgtacaccg atatgataaa |
| 61 | tatgttgata tttcaattga tagtatctta aatcagacat actctgactt tgagttaata |
| 121 | ataattgcaa atggagggga ttgtttcgag atagcaaaac agctgaagca ttatacagag |
| 181 | ctggataaca gagttaaaat ttatacatta gaaatagggc agttatcgtt tgcattaaat |
| 241 | tacgcagtaa ctaagtgtaa atactctatt attgccagaa tggattccga cgatgtttca |
| 301 | ctgccgttac gtctagaaaa acaatatatg tatatgttgc agaatgattt agaaatggtg |
| 361 | gggactggga tcagacttat caatgaaaac ggtgagttta ttaaagaatt aaaatatcca |
| 421 | aatcataata agataaataa gatacttcct tttaaaaatt gttttgcgca tcctactttg |
| 481 | atgttcaaga aagatgttat actaaagcag cgaggttatt gtggtggttt taattcagaa |
| 541 | gattatgatc tatggctcag aatcttaaat gaatgtccga atatacgctg ggataatcta |
| 601 | agtgagtgtt tgctaaatta tcgaattcat aacaaatcta cgcaaaaatc agcactcgca |
| 661 | tattatgaat gtgctagtta ttctctgcga gaattcttaa aaaaaagaac tattacgaat |
| 721 | tttctttctt gcctctatca tttttgtaaa gcactaataa aataa |

//
pTRC99Y
6866 bp ds-DNA
SEQ ID NO 6

| | |
|---|---|
| 1 | gtttgacagc ttatcatcga ctgcacggtg caccaatgct tctggcgtca ggcagccatc |
| 61 | ggaagctgtg gtatggctgt gcaggtcgta aatcactgca taattcgtgt cgctcaaggc |
| 121 | gcactcccgt tctggataat gttttttgcg ccgacatcat aacggttctg gcaaatattc |
| 181 | tgaaatgagc tgttgacaat taatcatccg gctcgtataa tgtgtggaat tgtgagcgga |
| 241 | taacaatttc acacaggaaa cagaccatgg aattcgagct cggtacccgg ggatcctcta |
| 301 | gagtcgacct gcaggcatgc aagcttggct gttttggcgg atgagagaag attttcagcc |
| 361 | tgatacagat taaatcagaa cgcagaagcg gtctgataaa acagaatttg cctggcggca |
| 421 | gtagcgcggt ggtcccacct gaccccatgc cgaactcaga agtgaaacgc cgtagcgccg |
| 481 | atggtagtgt ggggtctccc catgcgagag tagggaactg ccaggcatca aataaaacga |
| 541 | aaggctcagt cgaaagactg ggcctttcgt tttatctgtt gtttgtcggt gaacgctctc |
| 601 | ctgagtagga caaatccgcc gggagcggat ttgaacgttg cgaagcaacg gcccggaggg |
| 661 | tggcgggcag gacgcccgcc ataaactgcc aggcatcaaa ttaagcagaa ggccatcctg |
| 721 | acggatggcc tttttgcgtt tctacaaact ctttttgttt atttttctaa atacattcaa |
| 781 | atatgtatcc gctcatgaga caataaccct gataaatgct tcaataatat tgaaaaagga |
| 841 | agagtatgag tattcaacat ttccgtgtcg cccttattcc cttttttgcg gcattttgcc |
| 901 | ttcctgtttt tgctcaccca gaaacgctgg tgaaagtaaa agatgctgaa gatcagttgg |
| 961 | gtgcacgagt gggttacatc gaactggatc tcaacagcgg taagatcctt gagagttttc |
| 1021 | gccccgaaga acgttttcca atgatgagca cttttaaagt tctgctatgt ggcgcggtat |
| 1081 | tatcccgtgt tgacgccggg caagagcaac tcggtcgccg catacactat tctcagaatg |
| 1141 | acttggttga gtactcacca gtcacagaaa agcatcttac ggatggcatg acagtaagag |
| 1201 | aattatgcag tgctgccata accatgagtg ataacactgc ggccaactta cttctgacaa |
| 1261 | cgatcggagg accgaaggag ctaaccgctt ttttgcacaa catgggggat catgtaactc |
| 1321 | gccttgatcg ttgggaaccg gagctgaatg aagccatacc aaacgacgag cgtgacacca |
| 1381 | cgatgcctac agcaatggca acaacgttgc gcaaactatt aactggcgaa ctacttactc |
| 1441 | tagcttcccg gcaacaatta atagactgga tggaggcgga taaagttgca ggaccacttc |
| 1501 | tgcgctcggc ccttccggct ggctggttta ttgctgataa atctggagcc ggtgagcgtg |
| 1561 | ggtctcgcgg tatcattgca gcactggggc cagatggtaa gccctcccgt atcgtagtta |
| 1621 | tctacacgac ggggagtcag gcaactatgg atgaacgaaa tagacagatc gctgagatag |
| 1681 | gtgcctcact gattaagcat tggtaactgt cagaccaagt ttactcatat atactttaga |
| 1741 | ttgatttaaa acttcatttt taatttaaaa ggatctaggt gaagatcctt tttgataatc |
| 1801 | tcatgaccaa aatcccttaa cgtgagtttt cgttccactg agcgtcagac cccgtagaaa |
| 1861 | agatcaaagg atcttcttga gatccttttt ttctgcgcgt aatctgctgc ttgcaaacaa |
| 1921 | aaaaaccacc gctaccagcg gtggtttgtt tgccggatca agagctacca actctttttc |
| 1981 | cgaaggtaac tggcttcagc agagcgcaga taccaaatac tgtccttcta gtgtagccgt |
| 2041 | agttaggcca ccacttcaag aactctgtag caccgcctac atacctcgct ctgctaatcc |
| 2101 | tgttaccagt ggctgctgcc agtggcgata agtcgtgtct taccgggttg gactcaagac |
| 2161 | gatagttacc ggataaggcg cagcggtcgg gctgaacggg gggttcgtgc acacagccca |
| 2221 | gcttggagcg aacgacctac accgaactga gatacctaca gcgtgagcta tgagaaagcg |
| 2281 | ccacgcttcc cgaagggaga aaggcggaca ggtatccggt aagcggcagg gtcggaacag |
| 2341 | gagagcgcac gagggagctt ccagggggaa acgcctggta tctttatagt cctgtcgggt |
| 2401 | ttcgccacct ctgacttgag cgtcgatttt tgtgatgctc gtcagggggg cggagcctat |
| 2461 | ggaaaaacgc cagcaacgcg gcctttttac ggttcctggc cttttgctgg ccttttgctc |
| 2521 | acatgttctt tcctgcgtta tcccctgatt ctgtggataa ccgtattacc gcctttgagt |
| 2581 | gagctgatac cgctcgccgc agccgaacga ccgagcgcag cgagtcagtg agcgaggaag |
| 2641 | cggaagagcg cctgatgcgg tattttctcc ttacgcatct gtgcggtatt tcacaccgca |
| 2701 | tatgttgaag ctctaatttg tgagtttagt atacatgcat ttacttataa tacagttttt |
| 2761 | tagttttgct ggccgcatct tctcaaatat gcttcccagc ctgcttttct gtaacgttca |
| 2821 | ccctctacct tagcatccct tccctttgca aatagtcctc ttccaacaat aataatgtca |
| 2881 | gatcctgtag agaccacatc atccacggtt ctatactgtt gacccaatgc gtctcccttg |
| 2941 | tcatctaaac ccacaccggg tgtcataatc aaccaatcgt aaccttcatc tcttccaccc |
| 3001 | atgtctcttt gagcaataaa gccgataaca aaatctttgt cgctcttcgc aatgtcaaca |
| 3061 | gtacccttag tatattctcc agtagatagg gagcccttgc atgacaattc tgctaacatc |
| 3121 | aaaaggcctc taggttcctt tgttacttct tctgccgcct gcttcaaacc gctaacaata |
| 3181 | cctgggccca ccacaccgtg tgcattcgta atgtctgccc attctgctat tctgtataca |
| 3241 | cccgcagagt actgcaattt gactgtatta ccaatgtcag caaattttct gtcttcgaag |
| 3301 | agtaaaaaat tgtacttggc ggataatgcc tttagcggct taactgtgcc ctccatggaa |
| 3361 | aaatcagtca agatatccac atgtgttttt agtaaacaaa ttttgggacc taatgcttca |
| 3421 | actaactcca gtaattcctt ggtggtacga acatccaatg aagcacacaa gtttgtttgc |
| 3481 | ttttcgtgca tgatattaaa tagcttggca gcaacaggac taggatgagt agcagcacgt |
| 3541 | tccttatatg tagctttcga catgatttat cttcgtttcc tgcaggtttt tgttctgtgc |
| 3601 | agttgggtta agaatactgg gcaatttcat gtttcttcaa cactacatat gcgtatatat |
| 3661 | accaatctaa gtctgtgctc cttccttcgt tcttccttct gttcggagat taccgaatca |
| 3721 | aaaaaatttc aaagaaaccg aaatcaaaaa aaagaataaa aaaaaaatga tgaattgaat |
| 3781 | tgaaaagctg tggtatggtg cactctcagt acaatctgct ctgatgccgc atagttaagc |
| 3841 | cagccccgac acccgccaac acccgctgac gcgccctgac gggcttgtct gctcccggca |
| 3901 | tccgcttaca gacaagctgt gaccgtctcc gggagctgca tgtgtcagag gttttcaccg |
| 3961 | tcatcaccga aacgcgcgag acgaaagggc ctcgtgatac gcctattttt ataggttaat |
| 4021 | gtcatgataa taatggtttc ttagtatgat ccaatatcaa aggaaatgat agcattgaag |
| 4081 | gatgagacta atccaattga ggagtggcag catatagaac agctaaaggg tagtgctgaa |
| 4141 | ggaagcatac gataccccgc atggaatggg ataatatcac aggaggtact agactacctt |
| 4201 | tcatcctaca taaatagacg catataagta cgcatttaag cataaacacg cactatgccg |
| 4261 | ttcttctcat gtatatatat atacaggcaa cacgcagata taggtgcgac gtgaacagtg |
| 4321 | agctgtatgt gcgcagctcg cgttgcattt tcggaagcgc tcgttttcgg aaacgctttg |
| 4381 | aagttcctat tccgaagttc ctattctcta gaaagtatag gaacttcaga gcgcttttga |
| 4441 | aaaccaaaag cgctctgaag acgcactttc aaaaaaccaa aaacgcaccg gactgtaacg |
| 4501 | agctactaaa atattgcgaa taccgcttcc acaaacattg ctcaaaagta tctctttgct |
| 4561 | atatatctct gtgctatatc cctatataac ctacccatcc acctttcgct ccttgaactt |
| 4621 | gcatctaaac tcgacctcta cattttttat gtttatctct agtattactc tttagacaaa |
| 4681 | aaaattgtag taagaactat tcatagagtg aatcgaaaac aatacgaaaa tgtaaacatt |
| 4741 | tcctatacgt agtatataga gacaaaatag aagaaaccgt tcataatttt ctgaccaatg |
| 4801 | aagaatcatc aacgctatca ctttctgttc acaaagtatg cgcaatccac atcggtatag |
| 4861 | aatataatcg gggatgcctt tatcttgaaa aaatgcaccc gcagcttcgc tagtaatcag |
| 4921 | taaacgcggg aagtggagtc aggctttttt tatggaagag aaaatagaca ccaaagtagc |
| 4981 | cttcttctaa ccttaacgga cctacagtgc aaaaagttat caagagactg cattatagag |
| 5041 | cgcacaaagg agaaaaaaag taatctaaga tgctttgtta gaaaaatagc gctctcggga |
| 5101 | tgcatttttg tagaacaaaa aagaagtata gattctttgt tggtaaaata gcgctctcgc |
| 5161 | gttgcatttc tgttctgtaa aaatgcagct cagattcttt gtttgaaaaa ttagcgctct |
| 5221 | cgcgttgcat ttttgtttta caaaaatgaa gcacagattc ttcgttggta aaatagcgct |
| 5281 | ttcgcgttgc atttctgttc tgtaaaaatg cagctcagat tctttgtttg aaaaattagc |
| 5341 | gctctcgcgt tgcatttttg ttctacaaaa tgaagcacag atgcttcgtt cagggtgcac |
| 5401 | tctcagtaca atctgctctg atgccgcata gttaagccag tatacactcc gctatcgcta |
| 5461 | cgtgactggg tcatggctgc gccccgacac ccgccaacac ccgctgacgc gccctgacgg |
| 5521 | gcttgtctgc tcccggcatc cgcttacaga caagctgtga ccgtctccgg gagctgcatg |
| 5581 | tgtcagaggt tttcaccgtc atcaccgaaa cgcgcgaggc agcagatcaa ttcgcgcgcg |
| 5641 | aaggcgaagc ggcatgcatt tacgttgaca ccatcgaatg gtgcaaaacc tttcgcggta |
| 5701 | tggcatgata gcgcccggaa gagagtcaat tcagggtggt gaatgtgaaa ccagtaacgt |
| 5761 | tatacgatgt cgcagagtat gccggtgtct cttatcagac cgtttcccgc gtggtgaacc |
| 5821 | aggccagcca cgtttctgcg aaaacgcggg aaaaagtgga agcggcgatg gcggagctga |
| 5881 | attacattcc caaccgcgtg gcacaacaac tggcgggcaa acagtcgttg ctgattggcg |
| 5941 | ttgccacctc cagtctggcc ctgcacgcgc cgtcgcaaat tgtcgcggcg attaaatctc |
| 6001 | gcgccgatca actgggtgcc agcgtggtgg tgtcgatggt agaacgaagc ggcgtcgaag |
| 6061 | cctgtaaagc ggcggtgcac aatcttctcg cgcaacgcgt cagtgggctg atcattaact |
| 6121 | atccgctgga tgaccaggat gccattgctg tggaagctgc ctgcactaat gttccggcgt |
| 6181 | tatttcttga tgtctctgac cagacaccca tcaacagtat tattttctcc catgaagacg |
| 6241 | gtacgcgact gggcgtggag catctggtcg cattgggtca ccagcaaatc gcgctgttag |
| 6301 | cgggcccatt aagttctgtc tcggcgcgtc tgcgtctggc tggctggcat aaatatctca |
| 6361 | ctcgcaatca aattcagccg atagcggaac gggaaggcga ctggagtgcc atgtccggtt |
| 6421 | ttcaacaaac catgcaaatg ctgaatgagg gcatcgttcc cactgcgatg ctggttgcca |
| 6481 | acgatcagat ggcgctgggc gcaatgcgcg ccattaccga gtccgggctg cgcgttggtg |
| 6541 | cggatatctc ggtagtggga tacgacgata ccgaagacag ctcatgttat atcccgccgt |
| 6601 | caaccaccat caaacaggat tttcgcctgc tggggcaaac cagcgtggac cgcttgctgc |
| 6661 | aactctctca gggccaggcg gtgaagggca atcagctgtt gcccgtctca ctggtgaaaa |
| 6721 | gaaaaaccac cctggcgccc aatacgcaaa ccgcctctcc ccgcgcgttg gccgattcat |
| 6781 | taatgcagct ggcacgacag gtttcccgac tggaaagcgg gcagtgagcg caacgcaatt |
| 6841 | aatgtgagtt agcgcgaatt gatctg |

//
MBP-3TEV-Glucagon-4XGlycTag-6X-His (“6X-His” disclosed as SEQ ID NO: 36)
1416 bp ds-DNA
SEQ ID NO 7

| | |
|---|---|
| 1 | atgaaaaaga tttggctggc gctggctggt ttagttttag cgtttagcgc atcggcgtct |
| 61 | agaaaaatcg aagaaggtaa actggtaatc tggattaacg gcgataaagg ctataacggt |
| 121 | ctcgctgaag tcggtaagaa attcgagaaa gataccggaa ttaaagtcac cgttgagcat |
| 181 | ccggataaac tggaagagaa attcccacag gttgcggcaa ctggcgatgg ccctgacatt |
| 241 | atcttctggg cacacgaccg ctttggtggc tacgctcaat ctggcctgtt ggctgaaatc |
| 301 | accccggaca aagcgttcca ggacaagctg tatccgttta cctgggatgc cgtacgttac |
| 361 | aacggcaagc tgattgctta cccgatcgct gttgaagcgt tatcgctgat ttataacaaa |
| 421 | gatctgctgc cgaacccgcc aaaaacctgg gaagagatcc cggcgctgga taaagaactg |
| 481 | aaagcgaaag gtaagagcgc gctgatgttc aacctgcaag aaccgtactt cacctggccg |
| 541 | ctgattgctg ctgacggggg ttatgcgttc aagtatgaaa acggcaagta cgacattaaa |
| 601 | gacgtgggcg tggataacgc tggcgcgaaa gcgggtctga ccttcctggt tgacctgatt |
| 661 | aaaaacaaac acatgaatgc agacaccgat tactccatcg cagaagctgc ctttaataaa |
| 721 | ggcgaaacag cgatgaccat caacggcccg tgggcatggt ccaacatcga caccagcaaa |
| 781 | gtgaattatg gtgtaacggt actgccgacc ttcaagggtc aaccatccaa accgttcgtt |
| 841 | ggcgtgctga gcgcaggtat taacgccgcc agtccgaaca aagagctggc gaaagagttc |
| 901 | ctcgaaaact atctgctgac tgatgaaggt ctggaagcgg ttaataaaga caaaccgctg |
| 961 | ggtgccgtag cgctgaagtc ttacgaggaa gagttggcga aagatccacg tattgccgcc |
| 1021 | accatggaaa acgcccagaa aggtgaaatc atgccgaaca tcccgcagat gtccgctttc |
| 1081 | tggtatgccg tgcgtactgc ggtgatcaac gccgccagcg gtcgtcagac tgtcgatgaa |
| 1141 | gccctgaaag acgcgcagac tcgtatcacc aaggaaaacc tgtattttca gggcgaaaac |
| 1201 | ctgtattttc agggcgaaaa cctgtatttt cagggccact cacagggcac attcaccagt |
| 1261 | gactacagca agtacctgga ctccaggcgt gcccaggatt tcgtgcagtg gctgatgaat |
| 1321 | accaagagag atcagaacgc gaccgatcag aacgcgaccg atcagaacgc gaccgatcag |
| 1381 | aacgcgaccg tcgaccatca ccatcatcac cattaa |

//
MBP-3TEV-Glucagon-1XGlycTag-6X-His (“6X-His” disclosed as SEQ ID NO: 36)
1371 bp ds-DNA
SEQ ID NO 8

| | |
|---|---|
| 1 | atgaaaaaga tttggctggc gctggctggt ttagttttag cgtttagcgc atcggcgtct |
| 61 | agaaaaatcg aagaaggtaa actggtaatc tggattaacg gcgataaagg ctataacggt |
| 121 | ctcgctgaag tcggtaagaa attcgagaaa gataccggaa ttaaagtcac cgttgagcat |
| 181 | ccggataaac tggaagagaa attcccacag gttgcggcaa ctggcgatgg ccctgacatt |
| 241 | atcttctggg cacacgaccg ctttggtggc tacgctcaat ctggcctgtt ggctgaaatc |
| 301 | accccggaca aagcgttcca ggacaagctg tatccgttta cctgggatgc cgtacgttac |
| 361 | aacggcaagc tgattgctta cccgatcgct gttgaagcgt tatcgctgat ttataacaaa |
| 421 | gatctgctgc cgaacccgcc aaaaacctgg gaagagatcc cggcgctgga taaagaactg |
| 481 | aaagcgaaag gtaagagcgc gctgatgttc aacctgcaag aaccgtactt cacctggccg |
| 541 | ctgattgctg ctgacggggg ttatgcgttc aagtatgaaa acggcaagta cgacattaaa |
| 601 | gacgtgggcg tggataacgc tggcgcgaaa gcgggtctga ccttcctggt tgacctgatt |
| 661 | aaaaacaaac acatgaatgc agacaccgat tactccatcg cagaagctgc ctttaataaa |
| 721 | ggcgaaacag cgatgaccat caacggcccg tgggcatggt ccaacatcga caccagcaaa |
| 781 | gtgaattatg gtgtaacggt actgccgacc ttcaagggtc aaccatccaa accgttcgtt |
| 841 | ggcgtgctga gcgcaggtat taacgccgcc agtccgaaca aagagctggc gaaagagttc |
| 901 | ctcgaaaact atctgctgac tgatgaaggt ctggaagcgg ttaataaaga caaaccgctg |
| 961 | ggtgccgtag cgctgaagtc ttacgaggaa gagttggcga aagatccacg tattgccgcc |
| 1021 | accatggaaa acgcccagaa aggtgaaatc atgccgaaca tcccgcagat gtccgctttc |
| 1081 | tggtatgccg tgcgtactgc ggtgatcaac gccgccagcg gtcgtcagac tgtcgatgaa |
| 1141 | gccctgaaag acgcgcagac tcgtatcacc aaggaaaacc tgtattttca gggcgaaaac |
| 1201 | ctgtattttc agggcgaaaa cctgtatttt cagggccact cacagggcac attcaccagt |
| 1261 | gactacagca agtacctgga ctccaggcgt gcccaggatt tcgtgcagtg gctgatgaat |
| 1321 | accaagagag atcagaacgc gaccgtcgac catcaccatc atcaccatta a |

//
neuB YP_542350.1: N-acetylneuraminate synthase
EC 2.5.1.56
1041 bp ds-DNA
SEQ ID NO 9

| | |
|---|---|
| 1 | atgagtaata tatatatcgt tgctgaaatt ggttgcaacc ataatggtag tgttgatatt |
| 61 | gcaagagaaa tgatattaaa agccaaagag gccggtgtta atgcagtaaa attccaaaca |
| 121 | tttaaagctg ataaattaat ttcagctatt gcacctaagg cagagtatca aataaaaaac |
| 181 | acaggagaat tagaatctca gttagaaatg acaaaaaagc ttgaaatgaa gtatgacgat |
| 241 | tatctccatc taatggaata tgcagtcagt ttaaatttag atgttttttc tacccctttt |
| 301 | gacgaagact ctattgattt tttagcatct ttgaaacaaa aaatatggaa aatcccttca |
| 361 | ggtgagttat tgaatttacc gtatcttgaa aaaatagcca agcttccgat ccctgataag |
| 421 | aaaataatca tatcaacagg aatggctact attgatgaga taaaacagtc tgtttctatt |
| 481 | tttataaata ataaagttcc ggttgataat attacaatat tacattgcaa tactgaatat |
| 541 | ccaacgccct ttgaggatgt aaaccttaat gctattaatg atttgaaaaa acacttccct |
| 601 | aagaataaca taggcttctc tgatcattct agcgggtttt atgcagctat tgcggcggtg |
| 661 | ccttatggaa taacttttat tgaaaaacat ttcactttag ataaatctat gtctggccca |
| 721 | gatcatttgg cctcaataga acctgatgaa ctgaaacatc tatgtattgg ggtcaggtgt |
| 781 | gttgaaaaat ctttaggttc aaatagtaaa gtggttacag cttcagaaag gaagaataaa |
| 841 | atcgtagcaa gaaagtctat tatagctaaa acagagataa aaaaaggtga ggttttttca |
| 901 | gaaaaaaata taacaacaaa aagacctggt aatggtatca gtccgatgga gtggtataat |
| 961 | ttattgggta aaattgcaga gcaagacttt attccagatg aattaataat tcatagcgaa |
| 1021 | ttcaaaaatc agggggaata a |

//
neuA YP_542349.1: N-acetylneuraminate cytidylyltransferase
EC 2.7.7.43
1257 bp ds-DNA
SEQ ID NO 10

| | |
|---|---|
| 1 | atgagaacaa aaattattgc gataattcca gcccgtagtg gatctaaagg gttgagaaat |
| 61 | aaaaatgctt tgatgctgat agataaacct cttcttgctt atacaattga agctgccttg |
| 121 | cagtcagaaa tgtttgagaa agtaattgtg acaactgact ccgaacagta tggagcaata |
| 181 | gcagagtcat atggtgctga ttttttgctg agaccggaag aactagcaac tgataaagca |
| 241 | tcatcatttg aatttataaa acatgcgtta agtatatata ctgattatga gaactttgct |
| 301 | ttattacaac caacttcacc ctttagagat tcgacccata ttattgaggc tgtaaagtta |
| 361 | tatcaaactt tagaaaaata ccaatgtgtt gtttctgtta ctagaagcaa taagccatca |
| 421 | caaataatta gaccattaga tgattactcg acactgtctt tttttgacct tgattatagt |
| 481 | aaatataatc gaaactcaat agtagaatat catccgaatg gagctatatt tatagctaat |
| 541 | aagcagcatt atcttcatac aaagcatttt tttggtcgct attcactagc ttatattatg |
| 601 | gataaggaaa gctctttaga tatagatgat agaatggatt tcgaacttgc aattaccatt |
| 661 | cagcaaaaaa aaaatagaca aaaaatactt tatcaaaaca tacataatag aatcaatgag |
| 721 | aaacgaaatg aatttgatag tgtaagtgat ataactttaa ttggacactc gctgtttgat |
| 781 | tattgggacg taaaaaaaat aaatgatata gaagttaata acttaggtat cgctggtata |
| 841 | aactcgaagg agtactatga atatattatt gagaaagagc ggattgttaa tttcggagag |
| 901 | tttgttttca tcttttttgg aactaatgat atagttgtta gtgattggaa aaaagaagac |
| 961 | acattgtggt atttgaagaa aacatgccag tatataaaga agaaaaatgc tgcatcaaaa |
| 1021 | atttatttat tgtcggttcc tcctgttttt gggcgtattg atcgagataa tagaataatt |
| 1081 | aatgatttaa attcttatct tcgagagaat gtagattttg cgaagtttat tagcttggat |
| 1141 | cacgttttaa aagactctta tggcaatcta aataaaatgt atacttatga tggcttacat |
| 1201 | tttaatagta atgggtatac agtattagaa aacgaaatag cggagattgt taaatga |

//
neuC YP_542348.1: UDP-N-acetylglucosamine 2-epimerase
EC 5.1.3.14
1176 bp ds-DNA
SEQ ID NO 11

| | |
|---|---|
| 1 | atgaaaaaaa tattatacgt aactggatct agagctgaat atggaatagt tcggagactt |
| 61 | ttgacaatgc taagagaaac tccagaaata cagcttgatt tggcagttac aggaatgcat |
| 121 | tgtgataatg cgtatggaaa tacaatacat attatagaac aagataattt taatattatc |
| 181 | aaggttgtgg atataaatat caatacaact tcacatactc acattctcca ttcaatgagt |
| 241 | gtttgcctca attcgtttgg tgattttttt tcaaataaca catatgatgc ggttatggtt |
| 301 | ttaggcgata gatatgaaat attttcagtc gctatcgcag catcaatgca taatattcca |
| 361 | ttaattcata ttcatggtgg tgaaaagaca ttagctaatt atgatgagtt tattaggcat |
| 421 | tcaattacta aaatgagtaa actccatctt acttctacag aagagtataa aaaacgagta |
| 481 | attcaactag gtgaaaagcc tggtagtgtg tttaatattg gttctcttgg tgcagaaaat |
| 541 | gctctttcat tgcatttacc aaataagcag gagttggaac taaaatatgg ttcactgtta |
| 601 | aaacggtact ttgttgtagt attccatcct gaaacacttt ccacgcagtc ggttaatgat |
| 661 | caaatagatg agttattgtc agcgatttct ttttttaaaa atactcacga ctttattttt |
| 721 | attggcagta acgctgacac tggttctgat ataattcaga gaaaagtaaa atatttttgc |
| 781 | aaagagtata agttcagata tttgatttct attcgttcag aagattattt ggcaatgatt |
| 841 | aaatgctctt gtgggctaat tgggaactcc tcctctggtt taattgaggt tccatcttta |
| 901 | aaagttgcaa caattaacat tggtgatagg cagaaaggcc gtgttcgtgg agccagtgta |
| 961 | atagatgtac ccgttgaaaa aaatgcaatc gtcagaggga taaatatatc tcaagatgaa |
| 1021 | aaatttatta gtgttgtaca gtcatctagt aatccttatt ttaaagaaaa tgctttaatt |
| 1081 | aatgctgtta gaattattaa ggattttatt aaatcaaaaa ataaagatta caaagatttt |
| 1141 | tatgacatcc cggaatgtac caccagttat gactag |

//
1st: 2,3 NeuNAc transferase
N. meningitidis
EC 2.4.99.4
1116 bp ds-DNA
SEQ ID NO 12

| | |
|---|---|
| 1 | atgggcttga aaaaggcttg tttgaccgtg ttgtgtttga ttgttttttg tttcgggata |
| 61 | ttttatacat ttgaccgggt aaatcagggg gaaaggaatg cggtttccct gctgaaggag |
| 121 | aaacttttca atgaagaggg ggaaccggtc aatctgattt tctgttatac catattgcag |
| 181 | atgaaggtgg cggaaaggat tatggcgcag catccgggcg agcggtttta tgtggtgctg |
| 241 | atgtctgaaa acaggaatga aaaatacgat tattatttca atcagataaa ggataaggcg |
| 301 | gagcgggcgt actttttcca cctgccctac ggtttgaaca aatcgtttaa tttcattccg |
| 361 | acgatggcgg agctgaaggt aaagtcgatg ctgctgccga aagtcaagcg gatttatttg |
| 421 | gcaagtttgg aaaaagtcag cattgccgcc tttttgagca cttacccgga tgcggaaatc |
| 481 | aaaacctttg acgacgggac aggcaattta attcaaagca gcagctattt gggcgatgag |
| 541 | ttttctgtaa acgggacgat caagcggaat tttgcccgga tgatgatcgg agattggagc |
| 601 | atcgccaaaa cccgcaatgc ttccgacgag cattacacga tattcaaggg tttgaaaaac |
| 661 | attatggacg acggccgccg caagatgact tacctgccgc tgttcgatgc gtccgaactg |
| 721 | aagacggggg acgaaacggg cggcacggtg cggatacttt tgggttcgcc cgacaaagag |
| 781 | atgaaggaaa tttcggaaaa ggcggcaaaa aacttcaaaa tacaatatgt cgcgccgcat |
| 841 | ccccgccaaa cctacgggct ttccggcgta accacattaa attcgcccta tgtcatcgaa |
| 901 | gactatattt tgcgcgagat taagaaaaac ccgcatacga ggtatgaaat ttataccttt |
| 961 | ttcagcggcg cggcgttgac gatgaaggat tttcccaatg tgcacgttta cgcattgaaa |
| 1021 | ccggcttccc ttccggaaga ttattggctc aagccggtgt atgccctgtt tacccaatcc |
| 1081 | ggcatcccga ttttgacatt tgacgataaa aattaa |

//
neuD YP 542351.1: sialic acid biosynthesis protein, possible O-acetyltransferase
624 bp ds-DNA
SEQ ID NO 13

| | |
|---|---|
| 1 | atgagtaaaa aattaataat atttggtgcg ggtggttttt caaaatctat aattgacagc |
| 61 | ttaaatcata aacattacga gttaatagga tttatcgata aatataaaag tggttatcat |
| 121 | caatcatatc caatattagg taatgatatt gcagacatcg agaataagga taattattat |
| 181 | tattttattg ggataggcaa accatcaact aggaagcact atttaaacat cataagaaaa |
| 241 | cataatctac gcttaattaa cattatagat aaaactgcta ttctatcacc aaatattata |
| 301 | ctgggtgatg gaatttttat tggtaaaatg tgtatactta accgtgatac tagaatacat |
| 361 | gatgccgttg taataaatac taggagttta attgaacatg gtaatgaaat aggctgctgt |
| 421 | agcaatatct ctactaatgt tgtacttaat ggtgatgttt ctgttggaga agaaactttt |
| 481 | gttggtagct gtactgttgt aaatggccag ttgaagctag gctcaaagag tattattggt |
| 541 | tctgggtcgg ttgtaattag aaatatacca agtaatgttg tagttgctgg gactccaaca |
| 601 | agattaatta gggggaatga atga |

//
wbnK: α1,2 fucosyltransferase
E. coli O86
EC 2.4.1.69
909 bp ds-DNA
SEQ ID NO 14

| | |
|---|---|
| 1 | atgtatagtt gtttgtctgg tgggttaggt aatcaaatgt ttcagtatgc tgcggcatat |
| 61 | atcttacaga gaaagcttaa acaaagatca ttagttttag acgatagcta ttttttagat |
| 121 | tgctcaaatc gtgatacacg tagaagattt gaattgaatc aatttaacat atgttatgat |
| 181 | cgtctgacta caagtaagga aaaaaaagag atatccataa tacgacatgt aaatagatat |
| 241 | cgtttgccct tatttgttac aaattctata tttggagttc tactaaaaaa aaactatttg |
| 301 | cctgaagcaa aattttatga atttttgaac aactgtaaat tacaggttaa aaatggttat |
| 361 | tgtctatttt cttatttcca ggatgctaca ttgatagata gtcatcgtga tatgattctc |
| 421 | ccattattcc agattaatga agatttgctc aatttatgta atgacttgca tatttacaaa |
| 481 | aaagtgatat gtgagaatgc taacacaact tcactacata tcaggcgtgg agactacatc |
| 541 | accaaccctc acgcctctaa atttcatggg gtgttgccca tggattacta tgaaaaggct |
| 601 | attcgttata ttgaggatgt tcaaggagaa caggtgatta tcgtattttc agatgatgtg |
| 661 | aaatgggctg agaatacatt tgctaatcaa cctaattatt acgttgttaa taattctgaa |
| 721 | tgcgagtaca gtgcgattga tatgttttta atgtcaaagt gtaaaaacaa tataatagcc |
| 781 | aatagtacat atagttggtg gggggcatgg ttaaatactt tcgaagataa aatagttgtt |
| 841 | tcccctcgta agtggtttgc tggaaataat aaatctaagt tgaccatgga tagttggatt |
| 901 | aatctttga |

//
siaB: CMP-neuNAc synthetase (same function as NeuA)
EC 2.7.7.43
N. meningitidis
SEQ ID NO 15
atggaaaaacaaaatattgcggttatacttgcgcgccaaaactccaaaggattgccattaaaaaatctccgga
aaatgaatggcatatcattacttggtcatacaattaatgctgctatatcatcaaagtgttttgaccgcataat
tgtttcgactgatggcgggttaattgcagaagaagctaaaaatttcggtgtcgaagtcgtcctacgccctgca
gagctggcctccgatacagccagctctatttcaggtgtaatacatgctttagaaacaattggcagtaattccg
gcacagtaaccctattacaaccaaccagtccattacgcacaggggctcatattcgtgaagctttttctctatt
tgatgagaaaataaaaggatccgttgtctctgcatgcccaatggagcatcatccactaaaaaccctgcttcaa
atcaataatggcgaatatgcccccatgcgccatctaagcgatttggagcagcctcgccaacaattacctcagg
tcatttaggcctaatggtgcaatttacattaatgatactgcttcactaattgcaaataatgtttttttatcgc
cccaaccaaactttatattatgtctcatcaagactctatcgatattgatactgagcttgatttacaacaggca
gaaaacattcttaatcacaaggaaagctaa
cstII: bifunctional 2,3 2,8 neuNAc transferase
EC 2.4.99.4, 2.4.99.8
Campylobacter jejuni
SEQ ID NO 16
atgaaaaaagtgattattgccgggaatggtccttctctgaaagaaatcgactatagccgtctgccgaacgact
tcgacgtgtttcgctgtaaccagttctattttgaggacaaatattatctgggcaaaaaatgtaaagccgtgtt
ctataccccgaacttcttcttcgagcagtattatacgctgaaacatctgatccagaaccaggagtatgaaacc
gagctgatcatgtgtagcaactataaccaagcccacctggaaaacgaaaacttcgtgaaaaccttttatgact
atttccctgacgctcatctgggatatgatttcttcaaacagctgaaagagttcaacgcctatttcaaattcca
cgagatctattttaaccagcgtatcaccagcggtgtttatatgtgtgccgtggccattgctctgggttataaa
gagatttatctgagcggcatcgacttttatcagaacggttcctcctatgcctttgatacaaaacaggagaacc
tgctgaaactggcaccggatttcaaaaatgaccgctcccactatattggtcacagtaaaaacacggacattaa
agcgctggagtttctggagaaaacgtataaaatcaaactgtattgtctgtgcccgaattctctgctggcaaac
ttcattgagctggcgcctaatctgaacagcaacttcatcattcaggaaaaaaacaactatacgaaagacatcc
tgattccgagcagtgaagcatatggcaaattctcgaaaaacatcaacttcaaaaaaatcaaaatcaaagagaa
cgtctattataaactgattaaagatctgctgcgcctgcctagtgacatcaaacactatttcaaaggcaaataa
lic3b: bifunctional 2,3 2,8 neuNAc transferase
EC 2.4.99.-, 2.4.99.8
Haemophilus influenzae
SEQ ID NO 17
atgcccaatcaatcaatcaatcaatcaatcaatcaatcaatcaatcaatcaatcaatcaatcaatcaatcaat
caatcaatcaatcaatcaatcaatcaatcaatcaatcaaagcctgtcattattgcaggtaatggaacaagttt
aaaatcaattgactatagtttattacctaaagattatgatgttttccgttgcaatcaattttattttgaggat
cattattttcttggtaagaaaataaaaaaggtattttttaattgttctgtaatttttgaacaatactatacgt
ttatgcaattaattaaaaataatgaatatgaatatgctgatattattctatcatcttttctgaatttagggga
ttcagaattaaagaaaatccagcgtttagaaaaattactaccacaaatcgatcttggtcatagctatttaaaa
aaactacgagcttttgatgctcatttacaatatcacgaactatatgagaataagaggattacatcaggcgttt
atatgtgtgcagtggcaactgctatgggttataaagatctttatttgacaggcattgatttttatcaagaaaa
agggaatccttacgcatttcatcatcaaaaagaaaatattattaaattattaccttctttttcacaaaataaa
agtcaaaatgatatccattctatggaatatgatttaaatgcactttatttcttacaaaaacattatggtgtaa
atatttactgtatttcgccagaaagtcctctatgtaattattttcctttatcaccactgaataacccatttac
ttttattcccgaagaaaagaaaaattacacacaagatattttaattccgccagagtcagtgtataaaaaaatt
ggtatatattccaaaccaagaatttaccaaaatctggtttttcggttgatctgggatatattacgtttaccta
atgatataaaaaaagctttgaaagcaaagaaaatgagactacgcaaataa
lst: 2,3 neuNAc transferase
Neisseria meningitidis
EC 2.4.99.4
SEQ ID NO 18
atgggcttgaaaaaggcttgtttgaccgtgttgtgtttgattgttttttgtttcgggatattttatacatttg
accgggtaaatcagggggaaaggaatgcggtttccctgctgaaggagaaacttttcaatgaagagggggaacc
ggtcaatctgattttctgttataccatattgcagatgaaggtggcggaaaggattatggcgcagcatccgggc
gagcggttttatgtggtgctgatgtctgaaaacaggaatgaaaaatacgattattatttcaatcagataaagg
ataaggcggagcgggcgtactttttccacctgccctacggtttgaacaaatcgtttaatttcattccgacgat
ggcggagctgaaggtaaagtcgatgctgctgccgaaagtcaagcggatttatttggcaagtttggaaaaagtc
agcattgccgcctttttgagcacttacccggatgcggaaatcaaaacctttgacgacgggacaggcaatttaa
ttcaaagcagcagctatttgggcgatgagttttctgtaaacgggacgatcaagcggaattttgcccggatgat
gatcggagattggagcatcgccaaaacccgcaatgcttccgacgagcattacacgatattcaagggtttgaaa
aacattatggacgacggccgccgcaagatgacttacctgccgctgttcgatgcgtccgaactgaagacggggg
acgaaacgggcggcacggtgcggatacttttgggttcgcccgacaaagagatgaaggaaatttcggaaaaggc
ggcaaaaaacttcaaaatacaatatgtcgcgccgcatccccgccaaacctacgggctttccggcgtaaccaca
ttaaattcgccctatgtcatcgaagactatattttgcgcgagattaagaaaaacccgcatacgaggtatgaaa
tttatacctttttcagcggcgcggcgttgacgatgaaggattttcccaatgtgcacgtttacgcattgaaacc
ggcttcccttccggaagattattggctcaagccggtgtatgccctgtttacccaatccggcatcccgattttg
acatttgacgataaaaatta
neuS YP _542346.1: α2,8 polysialyltransferase
E. coli K1
EC 2.4.99.8
SEQ ID NO 19
atgatatttgatgctagtttaaagaagttgaggaaattatttgtaaatccaattgggtttttccgtgactcat
ggttttttaattctaaaaataagactgaaaagctattgtcacctttaaaaataaaaaacaaaaatatttttat
tgttgttcatttagggcaattaaagaaagcagagctttttatacaaaaatttagtaagcgtagtaattttctt
atcgtcttggcaactaaaaaaaacactgaaatgccaagattagttcttgagcaaatgaataaaaagttgtttt
cttcatataaactactatttataccaacagagccaaatacattttcgcttaaaaaagttatatggttttataa
tgtatataaatatatagttttaaattcaaaagctaaagatgcttattttatgagctatgcacaacattatgca
atcttcatatggttgttcaaaaaaaacaatataagatgttcattaattgaagaggggacagcgacgtataaaa
cagagaaaaaaaacacactagtaaatattaatttttattcgtggatcattaattcaattatcttgttccatta
tccagatttaaaatttgaaaatgtatacggcacctttccaaatttgttaaaagaaaaatttgatgcaaaaaaa
ttttttgagtttaaaactattccattagttaaatcgtcaacaagaatggataatctcatacataaatatcgta
tcactagagatgatattatatatgtaagtcaaagatattggattgacaacgaattgtatgcgcattcattaat
atctaccttgatgagaatagataaatctgataacgcaagagtttttataaaacctcaccctaaagaaactaaa
aaacatattaatgcaattcaaggtgcaataaataaagcaaagcgtcgtgatataattattattgtagaaaaag
actttttaatagagtcaataataaaaaaatgcaaaataaaacacttgattggattaacatcatcttctttggt
atacgcatctttagtttataaagagtgtaagacatattcaatagcacctattattataaaattgtgtaataat
gaaaaatcccaaaaagggactaatacgctgcgtctccatttcgatattttaaagaattttgataatgttaaaa
tattatcggatgatatatcatctccctctttgcacgataaaaggattttcttgggggagtaa
neuE YP_542347.1: function unknown. Possible primer for NeuS
SEQ ID NO 20
atgactagaaaaaaagtgattgttttgtctttcgttatgattctcattttttagctttgaaaaatatttttga
gcagatagatgttgattcatatgatttatttttttgctgcttggataattctctacaagagtttttaaaaaaa
aatttagatgaaaagatagttgtattctatcctgatgactttgtttattttttcacttttattaatattgagt
ttattttttgttcaacaggagggaaggaccttcatgaaattgttaatgctgtaagaacaaaaaatacaataat
tatatcttgttttccgggcattgtccttacttctcagatagaagcttttatttcaaaatctaatagtcactat
ttacttattaactcccctaaagacattaaaacgtataaaaaaatttgtaaaataataggggttccttttaatg
gaattctttttggtccaccatggattaaaaatgtcaatatcaatgcaaaaagtgagaattcttgtcttatcgt
tgatcaagttaatgagcccttgacgccaataaagaggatagaatatgcacgttttttgattagagtaattcag
aaacatccgcatatgaattttatttttaaaactcgaaatccttttatatcaccagagtcaattgtttttgata
ttaaggaatacattgaacgcttcgatttgaaaaatataacatttagcgatgataatattgattctttaatttc
taaagttgaatattgtattacaatatcttcttcggtcgcaatatattgtctggctaataaaattaaggtttat
ttaataaatggatttaatcatacctgcaatggacaatgttatttttcaagatctggacttattgttgattata
ataagtttaattttaaacacattccacgtattaaaaaaaaatggatggaggagaacctttattactctaggga
tattcaaaataagattttgaatgatattttaaaaatgccgccaaatgttaatgttagggcttttggaattaaa
agatctacattaattatattatttttgatctttttgaatttttttttctcattaggatcaaaaaaaataaaaa
cattgaaaaaaatccataaagttttattaaggtataagaaagatgatatttga
wbnH: GalNAc transferase (not required)
SEQ ID NO 21
atgaaaaatgttggttttattgttacaaaatcagaaattggtggtgcacaaacatgggtaaatgaaatatcta
accttattaaagaggaatgtaatatatttcttattacatctgaagaaggatggctcacacataaagatgtctt
tgccggagtttttgtcataccaggtattaaaaaatattttgacttccttacattgtttaaattgagaaaaatt
ttaaaagaaaataacatttcaacgttaatagcaagttctgctaatgccggagtttatgccaggttagttcgat
tactagtcgactttaaatgtatttatgtttcgcatggatggtcttgtttatataatggtggtcgcctaaaatc
aattttttgcattgttgaaaaatacctttctttattaactgatgttatatggtgtgtttccaaaagtgatgaa
aaaaaggcaattgagaatattggtataaaagaaccaaagataatcacagtatcgaattcagtgcctcagatgc
cgagatgtaataataaacaactccagtataaggttctgtttgttggtaggttaacacaccctaagcgccccga
attgttagcgaatgtaatatcgaaaaagccccagtatagcctccatatcgtaggagggggggaaaggttagaa
tcattgaagaaacaattcagtgaatgtgaaaatattcattttttgggtgaggtcaataatttttataactatc
atgagtatgatttattttcactgatatccgatagtgaaggtttgcctatgtcaggccttgaggctcacacagc
tgcaataccactcctgttaagtgatgtgggcggatgttttgaattaattgagggtaatgggttacttgtggaa
aatactgaagacgacattggatataaattggataaaatattcgatgactatgaaaattatcgggaacaggcaa
ttcgtgcctccgggaaatttgttatcgagaactatgcttcagcatataaaagcattattttaggttga
lgtE: gal transferase
SEQ ID NO 22
atgcaaaaccacgttatcagcttggcttccgccgcagagcgcagggcgcacattgccgataccttcggcagtc
gcggcatcccgttccagtttttcgacgcactgatgccgtctgaaaggctggaacgggcgatggcggaactcgt
ccccggcttgtcggcgcacccttatttgagcggagtggaaaaagcctgctttatgagccacgccgtattgtgg
gaacaggcgttggacgaaggcttaccgtatatcgccgtatttgaagatgatgtcttactcggcgaaggcgcgg
agcagttccttgccgaagatacttggctgcaagaacgctttgaccccgattccgcctttgtcgtccgcttgga
aacgatgtttatgcacgtcctgacctcgccctccggcgtggcggactacggcgggcgcgcctttccgcttttg
gaaagcgaacactgcgggacggcgggctatattatttcccgaaaggcgatgcgttttttcttggacaggtttg
ccgttttgccgcccgaacgcctgcaccctgtcgatttgatgatgttcggcaaccctgacgacagggaaggaat
gccggtttgccagctcaatcccgccttgtgcgcccaagagctgcattatgccaagtttctcagtcaaaacagt
atgttgggtagcgatttggaaaaagatagggaacaaggaagaagacaccgccgttcgttgaaggtgatgtttg
acttgaagcgtgctttgggtaaattcggtagggaaaagaagaaaagaatggagcgtcaaaggcaggcggagct
tgagaaagtttacggcaggcgggtcatattgttcaaatag
lgtB: β1,4 Galactosyltransferase
N. meningitidis
EC2.4.1.22
SEQ ID NO 23
atgcagaaccacgtgatttccctggcttcagcggccgagcgccgtgctcatattgctgccacctttggtagtc
gtggaatccctttccagttcttcgatgccctgatgccttcagaacgtctggagcaggcaatggcggagctggt
ccctggtctgtcagcccatccttatctgtctggcgttgaaaaagcgtgtttcatgtcccatgctgtcctgtgg
gaacaagccctggatgagggtctgccgtatatcgccgtgtttgaggacgatgtgctgctgggtgaaggtgctg
aacagtttctggccgaggacacttggctggaagagcgtttcgataaagactcagcgttcattgtccgtctgga
gacaatgtttatgcacgtgctgacttctccatctggtgtagccgattatggcggtcgtgcctttcctctgctg
gagtccgaacactgtggtacagccgggtatattatcagccgtaaagccatgcgtttctttctggatcgttttg
ctgtgctgcctccggagcgcctgcatcctgttgatctgatgatgtttggcaatcctgatgaccgtgagggtat
gccagtttgtcagctgaatccggcactgtgtgctcaggaactgcattatgccaaatttcacgaccagaatagc
gctctgggaagtctgattgaacatgatcgtcgcctgaaccgtaaacaacagtggcgtgatagtccggctaaca
cgtttaaacaccgcctgattcgtgctctgaccaaaattggccgtgagcgtgaaaaacgtcgtaaacgccgtga
acagacgattgggaaaatcattgtgccattccagtga
gne: aka z3206 UDP-N-acetylglucosamine 4-epimerase (from E. coli O157)
SEQ ID NO 24
Atgaacgataacgttttgctcataggagcttccggattcgtaggaacccgactacttgaaacggcaattgctg
actttaatatcaagaacctggacaaacagcagagccacttttatccagaaatcacacagattggtgatgttcg
tgatcaacaggcactcgaccaggcgttagccggttttgacactgttgtactactggcagcggaacaccgcgat
gacgtcagccctacttctctctattatgatgtcaacgttcagggtacccgcaatgtgctggcggccatggaaa
aaaatggcgttaaaaatatcatctttaccagttccgttgctgtttatggtttgaacaaacacaaccctgacga
aaaccatccacacgaccctttcaaccactacggcaaaagcaagtggcaggcggaggaagtgctgcgtgaatgg
tataacaaagcaccaacagaacgttcattaactatcatccgtcctaccgttatcttcggtgaacgcaaccgcg
gtaacgtctataacttgctgaaacagatcgctggcggcaagtttatgatggtgggcgcagggactaactataa
gtccatggcttatgttggaaacattgttgagtttatcaagtacaaactgaagaatgttgccgcaggttacgag
gtttataactacgttgataagccagacctgaacatgaaccagttggttgctgaagttgaacaaagcctgaaca
aaaagatcccttctatgcacttgccttacccactaggaatgctgggtggatattgattgatatcctgagcaaa
attacgggcaaaaaatacgctgtcagctctgtgcgcgtgaaaaaattctgcgcaacaacacagtttgacgcaa
cgaaagtgcattcttcaggttttgtggcaccgtatacgctgtcgcaaggtctggatcgaactctgcagtatga
attcgtccatgccaaaaaagacgacataacgtttgtttctgagtaa
neuD YP_542351.1
SEQ ID NO 25
MSKKLIIFGAGGFSKSIIDSLNHKHYELIGFIDKYKSGYHQSYPILGNDIADIENKDNYYYFIGIGKPSTRKH
YLNIIRKHNLRLINIIDKTAILSPNIILGDGIFIGKMCILNRDTRIHDAVVINTRSLIEHGNEIGCCSNISTN
VVLNGDVSVGEETFVGSCTVVNGQLKLGSKSIIGSGSVVIRNIPSNVVVAGTPTRLIRGNE*
neuB YP_542350.1
SEQ ID NO 26
MSNIYIVAEIGCNHNGSVDIAREMILKAKEAGVNAVKFQTFKADKLISAIAPKAEYQIKNTGELESQLEMTKK
LEMKYDDYLHLMEYAVSLNLDVFSTPFDEDSIDFLASLKQKIWKIPSGELLNLPYLEKIAKLPIPDKKIIIST
GMATIDEIKQSVSIFINNKVPVGNITILHCNTEYPTPFEDVNLNAINDLKKHFPKNNIGFSDHSSGFYAAIAA
VPYGITFIEKHFTLDKSMSGPDHLASIEPDELKHLCIGVRCVEKSLGSNSKVVTASERKNKIVARKSIIAKTE
IKKGEVFSEKNITTKRPGNGISPMEWYNLLGKIAEQDFIPDELIIHSEFKNQGE*
neuA YP_542349.1
SEQ ID NO 27
MRTKIIAIIPARSGSKGLRNKNALMLIDKPLLAYTIEAALQSEMFEKVIVTTDSEQYGAIAESYGADFLLRPE
ELATDKASSFEFIKHALSIYTDYESFALLQPTSPFRDSTHIIEAVKLYQTLEKYQCVVSVTRSNKPSQIIRPL
DDYSTLSFFDLDYSKYNRNSIVEYHPNGAIFIANKQHYLHTKHFFGRYSLAYIMDKESSLDIDDRMDFELAIT
IQQKKNRQKILYQNIHNRINEKRNEFDSVSDITLIGHSLFDYWDVKKINDIEVNNLGIAGINSKEYYEYIIEK
ELIVNFGEFVFIFFGTNDIVVSDWKKEDTLWYLKKTCQYIKKKNAASKIYLLSVPPVFGRIDRDNRIINDLNS
YLRENVDFAKFISLDHVLKDSYGNLNKMYTYDGLHFNSNGYTVLENEIAEIVK*
neuC YP_542348.1
SEQ ID NO 28
MKKILYVTGSRAEYGIVRRLLTMLRETPEIQLDLAVTGMHCDNAYGNTIHIIEQDNFNIIKVVDININTTSHT
HILHSMSVCLNSFGDFFSNNTYDAVMVLGDRYEIFSVAIAASMHNIPLIHIHGGEKTLANYDEFIRHSITKMS
KLHLTSTEEYKKRVIQLGEKPGSVFNIGSLGAENALSLHLPNKQELELKYGSLLKRYFVVVFHPETLSTQSVN
DQIDELLSAISFFKNTHDFIFIGSNADTGSDIIQRKVKYFCKEYKFRYLISIRSEDYLAMIKYSCGLIGNSSS
GLIEVPSLKVATINIGDRQKGRVRGASVIDVPVEKNAIVRGINISQDEKFISVVQSSSNPYFKENALINAVRI
IKDFIKSKNKDYKDFYDIPECTTSYD*
neuE YP_542347.1
SEQ ID NO 29
MTRKKVLCFVFRYDSHFLALKNIFEQIDVDSYDLFFCCLDNSLQEFVKKNLDEKIVVFYPDDFVCFFTFINIE
FIFCSTGGKDLHEIVNTVRTKDTIIISCFPGIVLTSQIEAFISKSNSHYLLINSPKDIKTYKKICKIIGVPFN
GILFGPPWIKNVNINAKSENSCLIVDQVNEPLTPIKRIEYARFLIRVIQKHPHMNFIFKTRNPLISPDSIVFD
IKEYIERFDLKNITFSDDNIDSLISKVEYCITISSSVAIYCLANKIKVYLINGFNHTCNGQCYFSRSGLIVDY
NKFNFKHIPRIKKKWMEENFYYSRDIQHKILNDILKMPSNVNVRTFGIKRSTLIILFLIFFNFFFSLGPKKIK
TLKKIHKVLLRYKKDDI*
neuS YP_542346.1
SEQ ID NO 30
MIFDASLKKLRKLFVNPIGFFRDSWFFNSKNKAEELLSPLKIKSKNIFIISNLGQLKKAESFVQKFSKRSNYL
IVLATEKNTEMPKIIVEQINNKLFSSYKVLFIPTFPNVFSLKKVIWFYNVYNYLVLNSKAKDAYFMSYAQHYA
IFVYLFKKNNIRCSLIEEGTGTYKTEKENPVVNINFYSEIINSIILFHYPDLKFENVYGTYPILLKKKFNAQK
FVEFKGAPSVKSSTRIDNVIHKYSITRDDIIYANQKYLIEHTLFADSLISILLRIDKPDNARIFIKPHPKEPK
KNINAIQKAIKKAKCRDIILITEPDFLIEPVIKKAKIKHLIGLTSSSLVYAPLVSKRCQSYSIAPLMIKLCDN
DKSQKGINTLRLHFDILKNFDNVKILSDDITSPSLHDKRIFLGE*
kpsS YP_542345.1
SEQ ID NO 31
atgcaaggtaatgcactaaccgttttattatccggtaaaaaatatctgctattgcaggggccgatgggacctt
ttttcaatgacgtcgccgaatggttagagtcattaggccgtaacgctgtgaatgttgtattcaacggtgggga
tcgtttttactgccgtcatcgacaatacctggcttactaccaaacgccgaaagagtttcccggatggttacgg
gatctccatcggcaatatgactttgataccatcctctgctttggtgactgccgcccattgcacaaagaagcaa
aacgctgggcaaagtcgaaagggatccgctttctggcatttgaggaaggatatttacgcccgcaatttattac
cgttgaagaagacggagtgaacgcatattcatcgctaccgcgcgatccggatttttatcgtaagttaccagat
atgcctacgccgcacgttgagaacttaaaaccttcaacgatgaaacgtataggtcatgcgatgtggtattacc
tgatgggctggcattaccgccatgagttccctcgctaccgccaccataaatcgttttccccctggtatgaagc
acgttgctgggttcgtgcatactggcgcaagcaactttacaaggtaacacagcgtaaggtattaccgaggtta
atgaacgagttggaccagcgttattatcttgccgttttgcaggtatataacgatagccagattcgtaaccaca
gcaattataacgatgtgcgtgactatattaatgaagtcatgtactcattttcacgtaaagcgccgaaagaaag
ttatttggtgatcaaacatcatccgatggatcgtggtcacagactctatcgaccattaattaagcggttaagt
aaggaatatggcttaagtgagcgcgtcatttatgtgcacgatctcccgatgccggaactattacgccacgcaa
aagcggtggtgacgattaacagtacggcggggatctctgcactgattcataacaaaccactcaaagtgatggg
caatgccctgtacgacatcaagggctgacgtatcaagggcatttgcaccagttctggcaggccgattttaaac
cggatatgaaactgtttaagaagtttcgggggtatttattgatgaagacgcaggttaattgggtttattatgg
ggggaacacaacaaactgccaacataatatatattaa
kpsC YP_542344.1
SEQ ID NO 32
atgattggcatttactcgcctggcatctggcgtattccgcatctggagaaatttctggcgcaaccgtgccaga
aactttctctgctgcgccctgttccgcaagaagttaatgctatcgccgtgtggggacatcgtcccagcgcggc
gaaaccagtcgccatcgccaaagcagcgggaaaacccgtcattcgtctggaagatggatttgtgcgttcgctg
gatcttggcgtcaatggcgagccgccgctttctctggtggtggatgattgtggcatttactacgatgccagca
agccttcggcgctggagaaactggtacaggataaagccggaaatacagctctgataagccaggccagagaagc
gatgcacaccatcgtgaccggggatatgtcgaaatataatctggcgcctgcgtttgtggctgatgagtcagaa
cgtacaaacatcgttctggttgtcgatcagacatttaatgatatgtcagtgacgtatggcaatgctggcccgc
atgagtttgctgccatgctggaagccgcgatggcggaaaatcctcaagccgaaatttgggtgaaggtgcaccc
agatgtactggaaggaaagaaaacaggttatttcgccgatctgcgcgccacgcaacgagtacgtttaattgcc
gagaatgtcagcccgcagtcgctgttgcgacacgtttcccgggtttacgtcgtgacctcccaatacggctttg
aagccttgctggcaggaaaaccagtaacatgtttcggccagccctggtatgcaagctggggcttaaccgacga
tcgccatccgcagtccgctttgttatctgcccgacgcggttctgccacgctggaggaactttttgccgctgca
tacctgcgttactgtcgctatatcgatccgcaaacgggagaagtaagcgatctatttaccgtgctgcaatggc
tgcaattacaacgtcgacatctgcaacagcgtaatggttatttatgggcgccaggcttaacgctgtggaagtc
ggcgatcctgaaacccttcttacgaacgccaacaaaccggctgagtttttcacgtcgctgtactgcggcgagc
gcctgcgtggtatggggtgtaaagggggaacagcaatggcgagccgaagcgcagcgaaaatcactgccattat
ggcgaatggaagatggttttctgcgttcatccggacttggctctgacctgctgccgccgctatcgttggtact
ggataaacgcgggatctactatgacgccacgcgccccagcgacctggaagtgctgcttaatcatagccagcta
acgctggcgcagcagatgcgagctgaaaaattacgccagcgactggttgaaagtaaactgagcaagtacaacc
tgggagccgatttctctctaccagccaaagccaaagataaaaaagttatcctggtgccgggtcaggtagagga
cgatgcctctattaaaacaggcacagtctcgattaagagcaaccttgagttattacgcacagtacgcgagcgt
aatccgcacgcctacattgtttataaaccgcacccggatgtactggtggggaatcgcaagggcgatattccgg
cagaactgactgctgaactcgctgattatcaggcactggacgccgatattattcaatgcattcagcgcgcaga
tgaagtgcataccatgacgtcgctgtcggggtttgaagcgttattacatggcaagcacgtacattgttacggc
ctgcccttctatgccggttggggtttaaccgtcgatgaacatcgttgcccgcgtcgcgagcgaaaattaacgt
tagcggatttgatctatcaggcgctgattgtttatccaacctatatccacccaacacggctacaacctattac
ggttgaagaggcggcggaatatttgatccagacaccgcgcaagccgatgtttattacccgaaaaaaagcgggg
cgagtaatacgttattaccgcaaattaattatgttctgtaaggtcagatttggctaa
kpsS YP_542345.1
SEQ ID NO 33
MQGNALTVLLSGKKYLLLQGPMGPFFNDVAEWLESLGRNAVNVVFNGGDRFYCRHRQYLAYYQTPKEFPGWLR
DLHRQYDFDTILCFGDCRPLHKEAKRWAKSKGIRFLAFEEGYLRPQFITVEEDGVNAYSSLPRDPDFYRKLPD
MPTPHVENLKPSTMKRIGHAMWYYLMGWHYRHEFPRYRHHKSFSPWYEARCWVRAYWRKQLYKVTQRKVLPRL
MNELDQRYYLAVLQVYNDSQIRNHSNYNDVRDYINEVMYSFSRKAPKESYLVIKHHPMDRGHRLYRPLIKRLS
KEYGLSERVIYVHDLPMPELLRHAKAVVTINSTAGISALIHNKPLKVMGNALYDIKGLTYQGHLHQFWQADFK
PDMKLFKKFRGYLLMKTQVNWVYYGGNTTNCQHNIY*
kpsC YP_542344.1
SEQ ID NO 34
MIGIYSPGIWRIPHLEKFLAQPCQKLSLLRPVPQEVNAIAVWGHRPSAAKPVAIAKAAGKPVIRLEDGFVRSL
DLGVNGEPPLSLVVDDCGIYYDASKPSALEKLVQDKAGNTALISQAREAMHTIVTGDMSKYNLAPAFVADESE
RTNIVLVVDQTFNDMSVTYGNAGPHEFAAMLEAAMAENPQAEIWVKVHPDVLEGKKTGYFADLRATQRVRLIA
ENVSPQSLLRHVSRVYVVTSQYGFEALLAGKPVTCFGQPWYASWGLTDDRHPQSALLSARRGSATLEELFAAA
YLRYCRYIDPQTGEVSDLFTVLQWLQLQRRHLQQRNGYLWAPGLTLWKSAILKPFLRTPTNRLSFSRRCTAAS
ACVVWGVKGEQQWRAEAQRKSLPLWRMEDGFLRSSGLGSDLLPPLSLVLDKRGIYYDATRPSDLEVLLNHSQL
TLAQQMRAEKLRQRLVESKLSKYNLGADFSLPAKAKDKKVILVPGQVEDDASIKTGTVSIKSNLELLRTVRER
NPHAYIVYKPHPDVLVGNRKGDIPAELTAELADYQALDADIIQCIQRADEVHTMTSLSGFEALLHGKHVHCYG
LPFYAGWGLTVDEHRCPRRERKLTLADLIYQALIVYPTYIHPTRLQPITVEEAAEYLIQTPRKPMFITRKKAG
RVIRYYRKLIMFCKVRFG*
pglB CAB73381.1
SEQ ID NO 35
MLKKEYLKNPYLVLFAMIVLAYVFSVFCRFYWVWWASEFNEYFFNNQLMIISNDGYAFAEGARDMIAGFHQPN
DLSYYGSSLSTLTYWLYKITPFSFESIILYMSTFLSSLVVIPIILLANEYKRPLMGFVAALLASVANSYYNRT
MSGYYDTDMLVIVLPMFILFFMVRMILKKDFFSLIALPLFIGIYLWWYPSSYTLNVALIGLFLIYTLIFHRKE
KIFYIAVILSSLTLSNIAWFYQSAIIVILFALFALEQKRLNFMIIGILGSATLIFLILSGGVDPILYQLKFYI
FRSDESANLTQGFMYFNVNQTIQEVENVDFSEFMRRISGSEIVFLFSLFGFVWLLRKHKSMIMALPILVLGFL
ALKGGLRFTIYSVPVMALGFGFLLSEFKAILVKKYSQLTSNVCIVFATILTLAPVFIHIYNYKAPTVFSQNEA
SLLNQLKNIANREDYVVTWWDYGYPVRYYSDVKTLVDGGKHLGKDNFFPSFSLSKDEQAAANMARLSVEYTEK
SFYAPQNDILKSDILQAMMKDYNQSNVDLFLASLSKPDFKIDTPKTRDIYLYMPARMSLIFSTVASFSFINLD
TGVLDKPFTFSTAYPLDVKNGEIYLSNGVVLSDDFRSFKIGDNVVSVNSIVEINSIKQGEYKITPIDDKAQFY
IFYLKDSAIPYAQFILMDKTMFNSAYVQMFFLGNYDKNLFDLVINSRDAKVFKLKI*

### REFERENCES

1. Walsh G (2000) Biopharmaceutical benchmarks. Nat Biotechnol 18: 831-833.
2. Walsh G (2003) Biopharmaceutical benchmarks—2003. Nat Biotechnol 21: 865-870.
3. Walsh G (2006) Biopharmaceutical benchmarks 2006. Nat Biotechnol 24: 769-776.
4. Pavlou A K, Belsey M J (2005) The therapeutic antibodies market to 2008. Eur J Pharm Biopharm 59: 389-396.
5. Pavlou A K, Reichert J M (2004) Recombinant protein therapeutics—success rates, market trends and values to 2010. Nat Biotechnol 22: 1513-1519.
6. Apweiler R, Hermjakob H, Sharon N (1999) On the frequency of protein glycosylation, as deduced from analysis of the SWISS-PROT database. Biochim Biophys Acta 1473: 4-8.
7. Helenius A, Aebi M (2001) Intracellular functions of N-linked glycans. Science 291: 2364-2369.
8. Choi B K, Bobrowicz P, Davidson R C, Hamilton S R, Kung D H, et al. (2003) Use of combinatorial genetic libraries to humanize N-linked glycosylation in the yeast Pichia pastoris. Proc Natl Acad Sci USA 100: 5022-5027.
9. Byrne B, Donohoe G G, O'Kennedy R (2007) Sialic acids: carbohydrate moieties that influence the biological and physical properties of biopharmaceutical proteins and living cells. Drug Discov Today 12: 319-326.
10. Harris J M, Chess R B (2003) Effect of pegylation on pharmaceuticals. Nat Rev Drug Discov 2: 214-221.
11. Veronese F M, Pasut G (2005) PEGylation, successful approach to drug delivery. Drug Discov Today 10: 1451-1458.
12. Graham M L (2003) Pegaspargase: a review of clinical studies. Adv Drug Deliv Rev 55: 1293-1302.
13. Levy Y, Hershfield M S, Fernandez-Mejia C, Polmar S H, Scudiery D, et al. (1988) Adenosine deaminase deficiency with late onset of recurrent infections: response to treatment with polyethylene glycol-modified adenosine deaminase. J Pediatr 113: 312-317.
14. DeFrees S, Wang Z G, Xing R, Scott A E, Wang J, et al. (2006) GlycoPEGylation of recombinant therapeutic proteins produced in Escherichia coli. Glycobiology 16: 833-843.
15. Caliceti P, Veronese F M (2003) Pharmacokinetic and biodistribution properties of poly(ethylene glycol)-protein conjugates. Adv Drug Deliv Rev 55: 1261-1277.
16. Whelan J (2005) Beyond PEGylation. Drug Discov Today 10: 301.
17. Vutskits L, Djebbara-Hannas Z, Zhang H, Paccaud J P, Durbec P, et al. (2001) PSA-NCAM modulates BDNF-dependent survival and differentiation of cortical neurons. Eur J Neurosci 13: 1391-1402.
18. Muhlenhoff M, Eckhardt M, Gerardy-Schahn R (1998) Polysialic acid: three-dimensional structure, biosynthesis and function. Curr Opin Struct Biol 8: 558-564.
19. Stockert R J (1995) The asialoglycoprotein receptor: relationships between structure, function, and expression. Physiol Rev 75: 591-609.
20. Steenbergen S M, Vimr E R (2003) Functional relationships of the sialyltransferases involved in expression of the polysialic acid capsules of Escherichia coli K1 and K92 and Neisseria meningitidis groups B or C. J Biol Chem 278: 15349-15359.
21. Fernandes A I, Gregoriadis G (2001) The effect of polysialylation on the immunogenicity and antigenicity of asparaginase: implication in its pharmacokinetics. Int J Pharm 217: 215-224.
22. Jain S, Hreczuk-Hirst D H, McCormack B, Mital M, Epenetos A, et al. (2003) Polysialylated insulin: synthesis, characterization and biological activity in vivo. Biochim Biophys Acta 1622: 42-49.
23. Constantinou A, Epenetos A A, Hreczuk-Hirst D, Jain S, Wright M, et al. (2009) Site-Specific Polysialylation of an Antitumor Single-Chain Fv Fragment. Bioconjug Chem.
24. Constantinou A, Epenetos A A, Hreczuk-Hirst D, Jain S, Deonarain MP (2008) Modulation of antibody pharmacokinetics by chemical polysialylation. Bioconjug Chem 19: 643-650.
25. Burda P, Aebi M (1999) The dolichol pathway of N-linked glycosylation. Biochim Biophys Acta 1426: 239-257.
26. Gavel Y, von Heijne G (1990) Sequence differences between glycosylated and non-glycosylated Asn-X-Thr/Ser acceptor sites: implications for protein engineering. Protein Eng 3: 433-442.
27. Kelleher D J, Gilmore R (2006) An evolving view of the eukaryotic oligosaccharyltransferase. Glycobiology 16: 47R-62R.
28. Szymanski C M, Yao R, Ewing C P, Trust T J, Guerry P (1999) Evidence for a system of general protein glycosylation in Campylobacter jejuni. Mol Microbiol 32: 1022-1030.
29. Linton D, Allan E, Karlyshev A V, Cronshaw A D, Wren B W (2002) Identification of N-acetylgalactosamine-containing glycoproteins PEB3 and CgpA in Campylobacter jejuni. Mol Microbiol 43: 497-508.
30. Young N M, Brisson J R, Kelly J, Watson D C, Tessier L, et al. (2002) Structure of the N-linked glycan present on multiple glycoproteins in the Gram-negative bacterium, Campylobacter jejuni. J Biol Chem 277: 42530-42539.
31. Feldman M F, Wacker M, Hernandez M, Hitchen P G, Marolda C L, et al. (2005) Engineering N-linked protein glycosylation with diverse O antigen lipopolysaccharide structures in Escherichia coli. Proc Natl Acad Sci USA 102: 3016-3021.
32. Alaimo C, Catrein I, Morf L, Marolda C L, Callewaert N, et al. (2006) Two distinct but interchangeable mechanisms for flipping of lipid-linked oligosaccharides. Embo J 25: 967-976.
33. Kelly J, Jarrell H, Millar L, Tessier L, Fiori L M, et al. (2006) Biosynthesis of the N-linked glycan in Campylobacter jejuni and addition onto protein through block transfer. J Bacteriol 188: 2427-2434.
34. Kowarik M, Young N M, Numao S, Schulz B L, Hug I, et al. (2006) Definition of the bacterial N-glycosylation site consensus sequence. Embo J 25: 1957-1966.
35. Wacker M, Linton D, Hitchen P G, Nita-Lazar M, Haslam S M, et al. (2002) N-linked glycosylation in Campylobacter jejuni and its functional transfer into E. coli. Science 298: 1790-1793.
36. Szymanski C M, Wren B W (2005) Protein glycosylation in bacterial mucosal pathogens. Nat Rev Microbiol 3: 225-237.
37. Weerapana E, Imperiali B (2006) Asparagine-linked protein glycosylation: from eukaryotic to prokaryotic systems. Glycobiology 16: 91R-101R.
38. Chen M M, Glover K J, Imperiali B (2007) From peptide to protein: comparative analysis of the substrate specificity of N-linked glycosylation in C. jejuni. Biochemistry 46: 5579-5585.
39. Kumamoto C A, Gannon P M (1988) Effects of Escherichia coli secB mutations on pre-maltose binding protein conformation and export kinetics. J Biol Chem 263: 11554-11558.
40. Schierle C F, Berkmen M, Huber D, Kumamoto C, Boyd D, et al. (2003) The DsbA signal sequence directs efficient, cotranslational export of passenger proteins to the Escherichia coli periplasm via the signal recognition particle pathway. J Bacteriol 185: 5706-5713.
41. Santini C L, Ize B, Chanal A, Muller M, Giordano G, et al. (1998) A novel sec-independent periplasmic protein translocation pathway in Escherichia coli. Embo J 17: 101-112.
42. Huber D, Boyd D, Xia Y, Olma M H, Gerstein M, et al. (2005) Use of thioredoxin as a reporter to identify a subset of Escherichia coli signal sequences that promote signal recognition particle-dependent translocation. J Bacteriol 187: 2983-2991.
43. DeLisa M P, Tullman D, Georgiou G (2003) Folding quality control in the export of proteins by the bacterial twin-arginine translocation pathway. Proc Natl Acad Sci USA 100: 6115-6120.
44. Sanders C, Wethkamp N, Lill H (2001) Transport of cytochrome c derivatives by the bacterial Tat protein translocation system. Mol Microbiol 41: 241-246.
45. Kowarik M, Numao S, Feldman M F, Schulz B L, Callewaert N, et al. (2006) N-linked glycosylation of folded proteins by the bacterial oligosaccharyltransferase. Science 314: 1148-1150.
46. Kuhlman B, Dantas G, Ireton G C, Varani G, Stoddard B L, et al. (2003) Design of a novel globular protein fold with atomic-level accuracy. Science 302: 1364-1368.
47. Cormack B P, Valdivia R H, Falkow S (1996) FACS-optimized mutants of the green fluorescent protein (GFP). Gene 173: 33-38.
48. Feilmeier B J, Iseminger G, Schroeder D, Webber H, Phillips G J (2000) Green fluorescent protein functions as a reporter for protein localization in Escherichia coli. J Bacteriol 182: 4068-4076.
49. Fisher A C, DeLisa M P (2008) Laboratory evolution of fast-folding green fluorescent protein using secretory pathway quality control. PLoS ONE 3: e2351.
50. Mazor Y, Van Blarcom T, Mabry R, Iverson B L, Georgiou G (2007) Isolation of engineered, full-length antibodies from libraries expressed in Escherichia coli. Nat Biotechnol 25: 563-565.
51. Simmons L C, Reilly D, Klimowski L, Raju T S, Meng G, et al. (2002) Expression of full-length immunoglobulins in Escherichia coli: rapid and efficient production of aglycosylated antibodies. J Immunol Methods 263: 133-147.
52. Raetz C R, Whitfield C (2002) Lipopolysaccharide endotoxins. Annu Rev Biochem 71: 635-700.
53. Goldberg J B, Hatano K, Meluleni G S, Pier G B (1992) Cloning and surface expression of Pseudomonas aeruginosa O antigen in Escherichia coli. Proc Natl Acad Sci USA 89: 10716-10720.
54. Dean C R, Franklund C V, Retief J D, Coyne M J, Jr., Hatano K, et al. (1999) Characterization of the serogroup O11 O -antigen locus of Pseudomonas aeruginosa PA103. J Bacteriol 181: 4275-4284.
55. Bugg T D, Brandish P E (1994) From peptidoglycan to glycoproteins: common features of lipid-linked oligosaccharide biosynthesis. FEMS Microbiol Lett 119: 255-262.
56. Feldman M F, Marolda C L, Monteiro M A, Perry M B, Parodi A J, et al. (1999) The activity of a putative polyisoprenol-linked sugar translocase (Wzx) involved in Escherichia coli O antigen assembly is independent of the chemical structure of the O repeat. J Biol Chem 274: 35129-35138.
57. Batchelor R A, Haraguchi G E, Hull R A, Hull S I (1991) Regulation by a novel protein of the bimodal distribution of lipopolysaccharide in the outer membrane of Escherichia coli. J Bacteriol 173: 5699-5704.
58. Wacker M, Feldman M F, Callewaert N, Kowarik M, Clarke B R, et al. (2006) Substrate specificity of bacterial oligosaccharyltransferase suggests a common transfer mechanism for the bacterial and eukaryotic systems. Proc Natl Acad Sci USA 103: 7088-7093.
59. Whitfield C (2006) Biosynthesis and assembly of capsular polysaccharides in Escherichia coli. Annu Rev Biochem 75: 39-68.
60. Steenbergen S M, Vimr E R (2008) Biosynthesis of the Escherichia coli K1 group 2 polysialic acid capsule occurs within a protected cytoplasmic compartment. Mol Microbiol 68: 1252-1267.
61. Andreishcheva E N, Vann W F (2006) Gene products required for de novo synthesis of polysialic acid in Escherichia coli K1. J Bacteriol 188: 1786-1797.
62. Datsenko K A, Wanner B L (2000) One-step inactivation of chromosomal genes in Escherichia coli K-12 using PCR products. Proc Natl Acad Sci USA 97: 6640-6645.
63. Bronner D, Sieberth V, Pazzani C, Roberts I S, Boulnois G J, et al. (1993) Expression of the capsular K5 polysaccharide of Escherichia coli: biochemical and electron microscopic analyses of mutants with defects in region 1 of the K5 gene cluster. J Bacteriol 175: 5984-5992.
64. Finke A, Bronner D, Nikolaev A V, Jann B, Jann K (1991) Biosynthesis of the Escherichia coli K5 polysaccharide, a representative of group II capsular polysaccharides: polymerization in vitro and characterization of the product. J Bacteriol 173: 4088-4094.
65. Troy F A, Vijay I K, Tesche N (1975) Role of undecaprenyl phosphate in synthesis of polymers containing sialic acid in Escherichia coli. J Biol Chem 250: 156-163.
66. Weisgerber C, Troy F A (1990) Biosynthesis of the polysialic acid capsule in Escherichia coli K1. The endogenous acceptor of polysialic acid is a membrane protein of 20 kDa. J Biol Chem 265: 1578-1587.
67. Chen M M, Weerapana E, Ciepichal E, Stupak J, Reid C W, et al. (2007) Polyisoprenol specificity in the Campylobacter jejuni N-linked glycosylation pathway. Biochemistry 46: 14342-14348.
68. Packiam M, Shell D M, Liu S V, Liu Y B, McGee D J, et al. (2006) Differential expression and transcriptional analysis of the alpha-2,3-sialyltransferase gene in pathogenic Neisseria spp. Infect Immun 74: 2637-2650.
69. Severi E, Hood D W, Thomas G H (2007) Sialic acid utilization by bacterial pathogens. Microbiology 153: 2817-2822.
70. Fox K L, Cox A D, Gilbert M, Wakarchuk W W, Li J, et al. (2006) Identification of a bifunctional lipopolysaccharide sialyltransferase in Haemophilus influenzae: incorporation of disialic acid. J Biol Chem 281: 40024-40032.
71. Gilbert M, Brisson J R, Karwaski M F, Michniewicz J, Cunningham A M, et al. (2000) Biosynthesis of ganglioside mimics in Campylobacter jejuni OH4384. Identification of the glycosyltransferase genes, enzymatic synthesis of model compounds, and characterization of nanomole amounts by 600-mhz (1)h and (13)c NMR analysis. J Biol Chem 275: 3896-3906.
72. Ali T, Weintraub A, Widmalm G (2006) Structural determination of the O-antigenic polysaccharide from the Shiga toxin-producing Escherichia coli 0171. Carbohydr Res 341: 1878-1883.
73. Hood D W, Cox A D, Gilbert M, Makepeace K, Walsh S, et al. (2001) Identification of a lipopolysaccharide alpha-2,3-sialyltransferase from Haemophilus influenzae. Mol Microbiol 39: 341-350.
74. Bouchet V, Hood D W, Li J, Brisson J R, Randle G A, et al. (2003) Host-derived sialic acid is incorporated into Haemophilus influenzae lipopolysaccharide and is a major virulence factor in experimental otitis media. Proc Natl Acad Sci USA 100: 8898-8903.
75. Baneyx F, Mujacic M (2004) Recombinant protein folding and misfolding in Escherichia coli. Nat Biotechnol 22: 1399-1408.
76. Georgiou G, Segatori L (2005) Preparative expression of secreted proteins in bacteria: status report and future prospects. Curr Opin Biotechnol 16: 538-545.
77. Simmons L C, Yansura D G (1996) Translational level is a critical factor for the secretion of heterologous proteins in Escherichia coli. Nat Biotechnol 14: 629-634.
78. Paschke M, Hohne W (2005) A twin-arginine translocation (Tat)-mediated phage display system. Gene 350: 79-88.
79. Thammawong P, Kasinrerk W, Turner R J, Tayapiwatana C (2006) Twin-arginine signal peptide attributes effective display of CD147 to filamentous phage. Appl Microbiol Biotechnol 69: 697-703.
80. Fisher A C, Kim J-Y, Tullman-Ercek D T, Henderson L A, DeLisa M P (2007) A universal strategy for the expression and purification of correctly folded proteins in Escherichia coli using the twin-arginine translocation system. Protein Sci: submitted.
81. Fisher A C, Kim W, DeLisa M P (2006) Genetic selection for protein solubility enabled by the folding quality control feature of the twin-arginine translocation pathway. Protein Sci 15: 449-458.
82. Kim J Y, Fogarty E A, Lu F J, Zhu H, Wheelock G D, et al. (2005) Twin-arginine translocation of active human tissue plasminogen activator in Escherichia coli. Appl Environ Microbiol 71: 8451-8459.
83. Waraho D, DeLisa M P (2007) A versatile selection technology for intracellular protein-protein interactions mediated by a unique bacterial hitchhiker transport mechanism. Proc Natl Acad Sci USA: submitted.
84. DeLisa M P, Samuelson P, Palmer T, Georgiou G (2002) Genetic analysis of the twin arginine translocator secretion pathway in bacteria. J Biol Chem 277: 29825-29831.
85. Tullman-Ercek D, DeLisa M P, Kawarasaki Y, Iranpour P, Ribnicky B, et al. (2007) Export pathway selectivity of Escherichia coli twin-arginine translocation signal peptides. J Biol. Chem.
86. DeLisa M P, Lee P, Palmer T, Georgiou G (2004) Phage shock protein PspA of Escherichia coli relieves saturation of protein export via the Tat pathway. J Bacteriol 186: 366-373.
87. Perez-Rodriguez R, Fisher A C, Perlmutter J D, Hicks M G, Chanal A, et al. (2007) An essential role for the DnaK molecular chaperone in stabilizing over-expressed substrate proteins of the bacterial twin-arginine translocation pathway. J Mol. Biol.

What is claimed is:

1. A method for producing an oligosaccharide composition comprising:
culturing a recombinant host cell to express one or more of the enzyme activites comprising:
a. GalNAc transferase (EC 2.4.1.-);
b. galactosyltransferase (EC 2.4.1.-);
c. fucosyltransferase (EC 2.4.1.69); and
d. sialyltransferase (EC 2.4.99.4, EC 2.4.99.-, EC 2.4.99.8).

2. The method of claim 1, wherein the culturing step comprises expressing a GalNAc transferase activity selected from α1,3-N-acetylgalactosamine transferase (EC 2.4.1-).

3. The method of claim 1, wherein the culturing step comprises expressing a galactosyltransferase selected from β1,3 galactosyl transferase (EC 2.4.1-) (WbnJ) and β1,4 galactosyltransferase (EC2.4.1.22).

4. The method of claim 1, wherein the culturing step comprises expressing a fucosyl transferase activity selected from α1,2 fucosyltransferase (EC 2.4.1.69).

5. The method of claim 1, wherein the culturing step comprises expressing a sialyltransferase activity selected from α2,3 NeuNAc transferase (EC 2.4.99.4), bifunctional α2,3 α2,8 neuNAc transferase (EC 2.4.99.-, EC 2.4.99.4, EC 2.4.99.8), and α2,8 polysialyltransferase (EC 2.4.99.8).

6. The method of claim 1, wherein the culturing step comprises expressing α1,3-N-acetylglucosaminyl transferase activity (EC 2.4.1-).

7. The method of claim 1, wherein the culturing step further comprises an attenuation in at least one of the enzyme activities selected from N-acetylneuraminate lyase (EC 4.1.3.3), undecaprenyl-phosphate glucose phosphotransferase (EC 2.7.8.-) and sialic acid aldolase activity.

8. The method of claim 1, wherein the culturing step further comprises one or more enzyme activites selected from UDP-GlcNAc transferase, flippase and oligosaccharyl transferase activity (EC 2.4.1.119).

9. The method of claim 1, wherein the culturing step produces of at least one oligosaccharide composition selected from human T, human sialyl T and human H antigen.

10. The method of claim 1, wherein the culturing step produces a polysialic acid.

11. The method of claim 1, wherein the culturing step further comprises expressing a protein of interest.

12. The method of claim 11, wherein the oligosaccharide composition is N-linked to the protein.

13. The method of claim 1 or 11, wherein the oligosaccharide composition is selected from
a. (Sia α2,8)ₙ-Sia α2,8-Sia α2,3-Galβ1,3-GalNAc α1,3-GalNAc α1,3-GlcNAc;
b. (Sia α2,8)ₙ-Sia α2,8-Sia α2,3-Galβ1,3-GalNAc α1,3-GlcNAc;
c. (Sia α2,8)ₙ-Sia α2,8-Sia α2,3-Galβ1,3-(GalNAc α1,3)ₙ;
d. Sia α2,3-Galβ1,3-GalNAc α1,3-GlcNAc;
e. Fuc α1,2-Galβ1,3-GalNAc α1,3-GlcNAc;
f. Galβ1,3-GalNAc α1,3-GlcNAc; and
g. Galβ1,3-GalNAc α1,3-GalNAc α1,3.

14. A host cell produced by any of the above claims.

15. An oligosaccharide composition produced by any of the above claims.

16. A glycoprotein composition produced by any of the above claims.

17. A recombinant host cell comprising at least one neu activity and at least one kps activity.

18. The host cell of claim 17 wherein the kps activity comprises kpsSCUDEF.

19. The host cell of claim 17 wherein the neu activity comprises neuDBACES.

20. The host cell of claim 17 further comprising neuCBA.

21. The host cell of claim 17 wherein one or more genes encoding kpsMT is attenuated.

22. The host cell of claim 17 wherein the neu activity comprises one or more of the following enzymes: NeuD (acetylase), NeuB (synthase), NeuA (synthase) NeuC (epimerase), NeuS and NeuS (polysialyltransferase).

23. The host cell of claim 1 further comprises introducing into the host a protein of interest.

24. The host cell of claim 1 further comprises an oligosaccharyl transferase activity.

25. A glycoprotein composition produced by any one of the above claims 17-24.

26. An oligosaccharide composition produced by any one of the above claims 17-24.


SEQUENCE LISTING (37 sequences)

SEQ ID NO: 1 - DNA, 7610 bp, Artificial Sequence
  Description of Artificial Sequence Synthetic polynucleotide
gatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatccttgcagcacatccccctttcgccagctggcgtaatagcgaagaggcccgcaccgatcgcccttcccaacagttgcgcagcctgaaaggcaggccgggccgtggtggccacggcctctaggccagatccagcggcatctgggttagtcgagcgcgggccgcttcccatgtctcaccagggcgagcctgtttcgcgatctcagcatctgaaatcttcccggccttgcgcttcgctggggccttacccaccgccttggcgggcttcttcggtccaaaactgaacaacagatgtgtgaccttgcgcccggtctttcgctgcgcccactccacctgtagcgggctgtgctcgttgatctgcgtcacggctggatcaagcactcgcaacttgaagtccttgatcgagggataccggccttccagttgaaaccactttcgcagctggtcaatttctatttcgcgctggccgatgctgtcccattgcatgagcagctcgtaaagcctgatcgcgtgggtgctgtccatcttggccacgtcagccaaggcgtatttggtgaactgtttggtgagttccgtcaggtacggcagcatgtctttggtgaacctgagttctacacggccctcaccctcccggtagatgattgtttgcacccagccggtaatcatcacactcggtcttttccccttgccattgggctcttgggttaaccggacttcccgccgtttcaggcgcagggccgcttctttgagctggttgtaggaagattcgatagggacacccgccatcgtcgctatgtcctccgccgtcactgaatacatcacttcatcggtgacaggctcgctcctcttcacctggctaatacaggccagaacgatccgctgttcctgaacactgaggcgatacgcggcctcgaccagggcattgcttttgtaaaccattgggggtgaggccacgttcgacattccttgtgtataaggggacactgtatctgcgtcccacaatacaacaaatccgtccctttacaacaacaaatccgtcccttcttaacaacaaatccgtcccttaatggcaacaaatccgtccctttttaaactctacaggccacggattacgtggcctgtagacgtcctaaaaggtttaaaagggaaaaggaagaaaagggtggaaacgcaaaaaacgcaccactacgtggccccgttggggccgcatttgtgcccctgaaggggcgggggaggcgtctgggcaatccccgttttaccagtcccctatcgccgcctgagagggcgcaggaagcgagtaatcagggtatcgaggcggattcacccttggcgtccaaccagcggcaccagcggctcgacaacccttaatataacttcgtataatgtatgctatacgaagttattaggtctagagatctgtttagcttgcctcgtccccgccgggtcagccggcggttaaggtatactttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgataagctccacgtgaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacat

SEQ ID NO: 2 - DNA, 987 bp, Campylobacter jejuni
atgaaaattcttattagcggtggtgcaggttatataggttctcatactttaagacaatttttaaaaacagatcatgaaatttgtgttttagataatctttctaagggttctaaaatcgcaatagaagatttgcaaaaaataagaacttttaaattttttgaacaagatttaagtgattttcaaggcgtaaaagcattgtttgagagagaaaaatttgacgctattgtgcattttgcagcgagcattgaagtttttgaaagtatgcaaaaccctttaaagtattatatgaataacactgttaatacgacaaatctcatcgaaacttgtttgcaaactggagtgaataaatttatattttcttcaacggcagccacttatggcgaaccacaaactcccgttgtgagcgaaacaagtcctttagcacctattaatccttatgggcgtagtaagcttatgagcgaagaggttttgcgtgatgcaagtatggcaaatcctgaatttaagcattgtattttaagatattttaatgttgcaggtgcttgcatggattatactttaggacaacgctatccaaaagcgactttgcttataaaagttgcagctgaatgtgccgcaggaaaacgtaataaacttttcatatttggcgatgattatgatacaaaagatggcacttgcataagagattttatccatgtggatgatatttcaagtgcgcatttatcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagggcttatttgtaaatctgcttttgattgggaaaaacagtgctaa

SEQ ID NO: 3 - DNA, 2142 bp, Campylobacter jejuni
atgttgaaaaaagagtatttaaaaaacccttatttagttttgtttgcgatgattatattagcttatgtttttagtgtattttgcaggttttattgggtttggtgggcaagtgagtttaatgagtattttttcaataatcagttaatgatcatttcaaatgatggctatgcttttgctgagggcgcaagagatatgatagcaggttttcatcagcctaatgatttgagttattatggatcttctttatccgcgcttacttattggctttataaaatcacacctttttcttttgaaagtatcattttatatatgagtacttttttatcttctttggtggtgattcctactattttgctagctaacgaatacaaacgtcctttaatgggctttgtagctgctcttttagcaagtatagcaaacagttattataatcgcactatgagtgggtattatgatacggatatgctggtaattgttttgcctatgtttattttattttttatggtaagaatgattttaaaaaaagactttttttcattgattgccttgccgttatttataggaatttatctttggtggtatccttcaagttatactttaaatgtagctttaattggactttttttaatttatacacttatttttcatagaaaagaaaagattttttatatagctgtgattttgtcttctcttactctttcaaatatagcatggttttatcaaagtgccattatagtaatactttttgctttattcgccttagagcaaaaacgcttaaattttatgattataggaattttaggtagtgcaactttgatatttttgattttaagtggtggggttgatcctatactttatcagcttaaattttatatttttagaagtgatgaaagtgcgaatttaacgcagggctttatgtattttaatgtcaatcaaaccatacaagaagttgaaaatgtagatcttagcgaatttatgcgaagaattagtggtagtgaaattgtttttttgttttctttgtttggttttgtatggcttttgagaaaacataaaagtatgattatggctttacctatattggtgcttgggtttttagccttaaaaggggggcttagatttaccatttattctgtacctgtaatggccttaggatttggttttttattgagcgagtttaaggctataatggttaaaaaatatagccaattaacttcaaatgtttgtattgtttttgcaactattttgactttagctccagtatttatccatatttacaactataaagcgccaacagttttttctcaaaatgaagcatcattattaaatcaattaaaaaatatagccaatagagaagattatgtggtaacttggtgggattatggttatcctgtgcgttattatagcgatgtgaaaactttagtagatggtggaaagcatttaggtaaggataattttttcccttcttttgctttaagcaaagatgaacaagctgcagctaatatggcaagacttagtgtagaatatacagaaaaaagcttttatgctccgcaaaatgatattttaaaaacagacattttgcaagccatgatgaaagattataatcaaagcaatgtggatttgtttctagcttcattatcaaaacctgattttaaaatcgatacgccaaaaactcgtgatatttatctttatatgcccgctagaatgtctttgattttttctacggtggctagtttttcttttattaatttagatacaggagttttggataaaccttttacctttagcacagcttatccacttgatgttaaaaatggagaaatttatcttagcaacggagtggttttaagcgatgattttagaagttttaaaataggtgataatgtggtttctgtaaatagtatcgtagagattaattctattaaacaaggtgaatacaaaatcactccaattgatgataaggctcagttttatattttttatttaaaggatagtgctattccttacgcacaatttattttaatggataaaaccatgtttaatagtgcttatgtgcaaatgttttttttaggaaattatgataagaatttatttgacttggtgattaattctagagatgctaaggtttttaaacttaaaatttaa

SEQ ID NO: 4 - DNA, 1131 bp, Campylobacter jejuni
atgagaataggatttttatcacatgcaggagcaagtatttatcattttagaatgcctattataaaagcattaaaagatagaaaagatgaagtttttgttatagtgccgcaagatgaatacacgcaaaaacttagagatcttggtttaaaagtaattgtttatgagttttcaagagctagtttaaatccttttgtagttttaaagaattttttttatcttgctaaggttttaaaaaatttaaatcttgatcttattcaaagtgcggcacacaaaagcaatacctttggaattttagcggcaaaatgggcaaaaattccttatcgttttgctttggtagaaggcttgggatctttttatatagatcaaggttttaaggcaaatttagtacgttttgttattaataatctttataaattaagttttaaatttgcacaccaatttatttttgtcaatgaaagtaatgccgagtttatgcggaatttaggacttaaggaaaataaaatttgtgtgataaaatccgtagggatcaatttaaaaaaattttttcctatttatatagaatcggaaaaaaaagagcttttttggagaaatttaaatatagataaaaaacctattgttcttatgatagcaagagctttatggcataaaggtgtaaaagaattttatgaaagtgctactatgctaaaagacaaagcaaattttgttttagttggtggaagagatgaaaatccttcttgtgcgagtttggagtttttaaactcgggtgtggtgcattatttgggtgctagaagtgatatagtcgagcttttgcaaaattgtgatatttttgttttaccaagctataaagaaggctttcctgtaagtgttttggaggcaaaagcttgtggcaaggctatagtggtgagtgattgtgaaggttgtgtagaggctatttctaatgcttatgatggactttgggcaaaaacaaaaaatgctaaggatttaagcgaaaaaatttcacttttattagaagatgaaaaattaagattaaatttagctaaaaatgctgcccaagatgctttacaatacgatgaaaataatatcgcacagcgttatttaaaactttatgatagggtaattaagaatgtatga

SEQ ID NO: 5 - DNA, 765 bp, Escherichia coli
atgtcattgagaatattagatatgatttcagtaataatggctgtacaccgatatgataaatatgttgatatttcaattgatagtatcttaaatcagacatactctgactttgagttaataataattgcaaatggaggggattgtttcgagatagcaaaacagctgaagcattatacagagctggataacagagttaaaatttatacattagaaatagggcagttatcgtttgcattaaattacgcagtaactaagtgtaaatactctattattgccagaatggattccgacgatgtttcactgccgttacgtctagaaaaacaatatatgtatatgttgcagaatgatttagaaatggtggggactgggatcagacttatcaatgaaaacggtgagtttattaaagaattaaaatatccaaatcataataagataaataagatacttccttttaaaaattgttttgcgcatcctactttgatgttcaagaaagatgttatactaaagcagcgaggttattgtggtggttttaattcagaagattatgatctatggctcagaatcttaaatgaatgtccgaatatacgctgggataatctaagtgagtgtttgctaaattatcgaattcataacaaatctacgcaaaaatcagcactcgcatattatgaatgtgctagttattctctgcgagaattcttaaaaaaaagaactattacgaattttctttcttgcctctatcatttttgtaaagcactaataaaataa

SEQ ID NO: 6 - DNA, 6866 bp, Artificial Sequence
  Description of Artificial Sequence Synthetic polynucleotide
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgttgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcagggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 7 - DNA, 1416 bp, Artificial Sequence
  Description of Artificial Sequence Synthetic polynucleotide
atgaaaaagatttggctggcgctggctggtttagttttagcgtttagcgcatcggcgtctagaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaaggaaaacctgtattttcagggcgaaaacctgtattttcagggcgaaaacctgtattttcagggccactcacagggcacattcaccagtgactacagcaagtacctggactccaggcgtgcccaggatttcgtgcagtggctgatgaataccaagagagatcagaacgcgaccgatcagaacgcgaccgatcagaacgcgaccgatcagaacgcgaccgtcgaccatcaccatcatcaccattaa

SEQ ID NO: 8 - DNA, 1371 bp, Artificial Sequence
  Description of Artificial Sequence Synthetic polynucleotide
atgaaaaagatttggctggcgctggctggtttagttttagcgtttagcgcatcggcgtctagaaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaaggaaaacctgtattttcagggcgaaaacctgtattttcagggcgaaaacctgtattttcagggccactcacagggcacattcaccagtgactacagcaagtacctggactccaggcgtgcccaggatttcgtgcagtggctgatgaataccaagagagatcagaacgcgaccgtcgaccatcaccatcatcaccattaa

SEQ ID NO: 9 - DNA, 1041 bp, Escherichia coli
atgagtaatatatatatcgttgctgaaattggttgcaaccataatggtagtgttgatattgcaagagaaatgatattaaaagccaaagaggccggtgttaatgcagtaaaattccaaacatttaaagctgataaattaatttcagctattgcacctaaggcagagtatcaaataaaaaacacaggagaattagaatctcagttagaaatgacaaaaaagcttgaaatgaagtatgacgattatctccatctaatggaatatgcagtcagtttaaatttagatgttttttctaccccttttgacgaagactctattgattttttagcatctttgaaacaaaaaatatggaaaatcccttcaggtgagttattgaatttaccgtatcttgaaaaaatagccaagcttccgatccctgataagaaaataatcatatcaacaggaatggctactattgatgagataaaacagtctgtttctatttttataaataataaagttccggttgataatattacaatattacattgcaatactgaatatccaacgccctttgaggatgtaaaccttaatgctattaatgatttgaaaaaacacttccctaagaataacataggcttctctgatcattctagcgggttttatgcagctattgcggcggtgccttatggaataacttttattgaaaaacatttcactttagataaatctatgtctggcccagatcatttggcctcaatagaacctgatgaactgaaacatctatgtattggggtcaggtgtgttgaaaaatctttaggttcaaatagtaaagtggttacagcttcagaaaggaagaataaaatcgtagcaagaaagtctattatagctaaaacagagataaaaaaaggtgaggttttttcagaaaaaaatataacaacaaaaagacctggtaatggtatcagtccgatggagtggtataatttattgggtaaaattgcagagcaagactttattccagatgaattaataattcatagcgaattcaaaaatcagggggaataa

SEQ ID NO: 10 - DNA, 1257 bp, Escherichia coli
atgagaacaaaaattattgcgataattccagcccgtagtggatctaaagggttgagaaataaaaatgctttgatgctgatagataaacctcttcttgcttatacaattgaagctgccttgcagtcagaaatgtttgagaaagtaattgtgacaactgactccgaacagtatggagcaatagcagagtcatatggtgctgattttttgctgagaccggaagaactagcaactgataaagcatcatcatttgaatttataaaacatgcgttaagtatatatactgattatgagaactttgctttattacaaccaacttcaccctttagagattcgacccatattattgaggctgtaaagttatatcaaactttagaaaaataccaatgtgttgtttctgttactagaagcaataagccatcacaaataattagaccattagatgattactcgacactgtctttttttgaccttgattatagtaaatataatcgaaactcaatagtagaatatcatccgaatggagctatatttatagctaataagcagcattatcttcatacaaagcatttttttggtcgctattcactagcttatattatggataaggaaagctctttagatatagatgatagaatggatttcgaacttgcaattaccattcagcaaaaaaaaaatagacaaaaaatactttatcaaaacatacataatagaatcaatgagaaacgaaatgaatttgatagtgtaagtgatataactttaattggacactcgctgtttgattattgggacgtaaaaaaaataaatgatatagaagttaataacttaggtatcgctggtataaactcgaaggagtactatgaatatattattgagaaagagcggattgttaatttcggagagtttgttttcatcttttttggaactaatgatatagttgttagtgattggaaaaaagaagacacattgtggtatttgaagaaaacatgccagtatataaagaagaaaaatgctgcatcaaaaatttatttattgtcggttcctcctgtttttgggcgtattgatcgagataatagaataattaatgatttaaattcttatcttcgagagaatgtagattttgcgaagtttattagcttggatcacgttttaaaagactcttatggcaatctaaataaaatgtatacttatgatggcttacattttaatagtaatgggtatacagtattagaaaacgaaatagcggagattgttaaatga

SEQ ID NO: 11 - DNA, 1176 bp, Escherichia coli
atgaaaaaaatattatacgtaactggatctagagctgaatatggaatagttcggagacttttgacaatgctaagagaaactccagaaatacagcttgatttggcagttacaggaatgcattgtgataatgcgtatggaaatacaatacatattatagaacaagataattttaatattatcaaggttgtggatataaatatcaatacaacttcacatactcacattctccattcaatgagtgtttgcctcaattcgtttggtgattttttttcaaataacacatatgatgcggttatggttttaggcgatagatatgaaatattttcagtcgctatcgcagcatcaatgcataatattccattaattcatattcatggtggtgaaaagacattagctaattatgatgagtttattaggcattcaattactaaaatgagtaaactccatcttacttctacagaagagtataaaaaacgagtaattcaactaggtgaaaagcctggtagtgtgtttaatattggttctcttggtgcagaaaatgctctttcattgcatttaccaaataagcaggagttggaactaaaatatggttcactgttaaaacggtactttgttgtagtattccatcctgaaacactttccacgcagtcggttaatgatcaaatagatgagttattgtcagcgatttctttttttaaaaatactcacgactttatttttattggcagtaacgctgacactggttctgatataattcagagaaaagtaaaatatttttgcaaagagtataagttcagatatttgatttctattcgttcagaagattatttggcaatgattaaatgctcttgtgggctaattgggaactcctcctctggtttaattgaggttccatctttaaaagttgcaacaattaacattggtgataggcagaaaggccgtgttcgtggagccagtgtaatagatgtacccgttgaaaaaaatgcaatcgtcagagggataaatatatctcaagatgaaaaatttattagtgttgtacagtcatctagtaatccttattttaaagaaaatgctttaattaatgctgttagaattattaaggattttattaaatcaaaaaataaagattacaaagatttttatgacatcccggaatgtaccaccagttatgactag

SEQ ID NO: 12 - DNA, 1116 bp, Neisseria meningitidis
atgggcttgaaaaaggcttgtttgaccgtgttgtgtttgattgttttttgtttcgggatattttatacatttgaccgggtaaatcagggggaaaggaatgcggtttccctgctgaaggagaaacttttcaatgaagagggggaaccggtcaatctgattttctgttataccatattgcagatgaaggtggcggaaaggattatggcgcagcatccgggcgagcggttttatgtggtgctgatgtctgaaaacaggaatgaaaaatacgattattatttcaatcagataaaggataaggcggagcgggcgtactttttccacctgccctacggtttgaacaaatcgtttaatttcattccgacgatggcggagctgaaggtaaagtcgatgctgctgccgaaagtcaagcggatttatttggcaagtttggaaaaagtcagcattgccgcctttttgagcacttacccggatgcggaaatcaaaacctttgacgacgggacaggcaatttaattcaaagcagcagctatttgggcgatgagttttctgtaaacgggacgatcaagcggaattttgcccggatgatgatcggagattggagcatcgccaaaacccgcaatgcttccgacgagcattacacgatattcaagggtttgaaaaacattatggacgacggccgccgcaagatgacttacctgccgctgttcgatgcgtccgaactgaagacgggggacgaaacgggcggcacggtgcggatacttttgggttcgcccgacaaagagatgaaggaaatttcggaaaaggcggcaaaaaacttcaaaatacaatatgtcgcgccgcatccccgccaaacctacgggctttccggcgtaaccacattaaattcgccctatgtcatcgaagactatattttgcgcgagattaagaaaaacccgcatacgaggtatgaaatttatacctttttcagcggcgcggcgttgacgatgaaggattttcccaatgtgcacgtttacgcattgaaaccggcttcccttccggaagattattggctcaagccggtgtatgccctgtttacccaatccggcatcccgattttgacatttgacgataaaaattaa

SEQ ID NO: 13 - DNA, 624 bp, Escherichia coli
atgagtaaaaaattaataatatttggtgcgggtggtttttcaaaatctataattgacagcttaaatcataaacattacgagttaataggatttatcgataaatataaaagtggttatcatcaatcatatccaatattaggtaatgatattgcagacatcgagaataaggataattattattattttattgggataggcaaaccatcaactaggaagcactatttaaacatcataagaaaacataatctacgcttaattaacattatagataaaactgctattctatcaccaaatattatactgggtgatggaatttttattggtaaaatgtgtatacttaaccgtgatactagaatacatgatgccgttgtaataaatactaggagtttaattgaacatggtaatgaaataggctgctgtagcaatatctctactaatgttgtacttaatggtgatgtttctgttggagaagaaacttttgttggtagctgtactgttgtaaatggccagttgaagctaggctcaaagagtattattggttctgggtcggttgtaattagaaatataccaagtaatgttgtagttgctgggactccaacaagattaattagggggaatgaatga

SEQ ID NO: 14 - DNA, 909 bp, Escherichia coli
atgtatagttgtttgtctggtgggttaggtaatcaaatgtttcagtatgctgcggcatatatcttacagagaaagcttaaacaaagatcattagttttagacgatagctattttttagattgctcaaatcgtgatacacgtagaagatttgaattgaatcaatttaacatatgttatgatcgtctgactacaagtaaggaaaaaaaagagatatccataatacgacatgtaaatagatatcgtttgcccttatttgttacaaattctatatttggagttctactaaaaaaaaactatttgcctgaagcaaaattttatgaatttttgaacaactgtaaattacaggttaaaaatggttattgtctattttcttatttccaggatgctacattgatagatagtcatcgtgatatgattctcccattattccagattaatgaagatttgctcaatttatgtaatgacttgcatatttacaaaaaagtgatatgtgagaatgctaacacaacttcactacatatcaggcgtggagactacatcaccaaccctcacgcctctaaatttcatggggtgttgcccatggattactatgaaaaggctattcgttatattgaggatgttcaaggagaacaggtgattatcgtattttcagatgatgtgaaatgggctgagaatacatttgctaatcaacctaattattacgttgttaataattctgaatgcgagtacagtgcgattgatatgtttttaatgtcaaagtgtaaaaacaatataatagccaatagtacatatagttggtggggggcatggttaaatactttcgaagataaaatagttgtttcccctcgtaagtggtttgctggaaataataaatctaagttgaccatggatagttggattaatctttga

SEQ ID NO: 15 - DNA, 687 bp, Neisseria meningitidis
atggaaaaacaaaatattgcggttatacttgcgcgccaaaactccaaaggattgccattaaaaaatctccggaaaatgaatggcatatcattacttggtcatacaattaatgctgctatatcatcaaagtgttttgaccgcataattgtttcgactgatggcgggttaattgcagaagaagctaaaaatttcggtgtcgaagtcgtcctacgccctgcagagctggcctccgatacagccagctctatttcaggtgtaatacatgctttagaaacaattggcagtaattccggcacagtaaccctattacaaccaaccagtccattacgcacaggggctcatattcgtgaagctttttctctatttgatgagaaaataaaaggatccgttgtctctgcatgcccaatggagcatcatccactaaaaaccctgcttcaaatcaataatggcgaatatgcccccatgcgccatctaagcgatttggagcagcctcgccaacaattacctcaggcatttaggcctaatggtgcaatttacattaatgatactgcttcactaattgcaaataattgtttttttatcgccccaaccaaactttatattatgtctcatcaagactctatcgatattgatactgagcttgatttacaacaggcagaaaacattcttaatcacaaggaaagctaa

SEQ ID NO: 16 - DNA, 876 bp, Campylobacter jejuni
atgaaaaaagtgattattgccgggaatggtccttctctgaaagaaatcgactatagccgtctgccgaacgacttcgacgtgtttcgctgtaaccagttctattttgaggacaaatattatctgggcaaaaaatgtaaagccgtgttctataccccgaacttcttcttcgagcagtattatacgctgaaacatctgatccagaaccaggagtatgaaaccgagctgatcatgtgtagcaactataaccaagcccacctggaaaacgaaaacttcgtgaaaaccttttatgactatttccctgacgctcatctgggatatgatttcttcaaacagctgaaagagttcaacgcctatttcaaattccacgagatctattttaaccagcgtatcaccagcggtgtttatatgtgtgccgtggccattgctctgggttataaagagatttatctgagcggcatcgacttttatcagaacggttcctcctatgcctttgatacaaaacaggagaacctgctgaaactggcaccggatttcaaaaatgaccgctcccactatattggtcacagtaaaaacacggacattaaagcgctggagtttctggagaaaacgtataaaatcaaactgtattgtctgtgcccgaattctctgctggcaaacttcattgagctggcgcctaatctgaacagcaacttcatcattcaggaaaaaaacaactatacgaaagacatcctgattccgagcagtgaagcatatggcaaattctcgaaaaacatcaacttcaaaaaaatcaaaatcaaagagaacgtctattataaactgattaaagatctgctgcgcctgcctagtgacatcaaacactatttcaaaggcaaataa

SEQ ID NO: 17 - DNA, 999 bp, Haemophilus influenza
atgcccaatcaatcaatcaatcaatcaatcaatcaatcaatcaatcaatcaatcaatcaatcaatcaatcaatcaatcaatcaatcaatcaatcaatcaatcaatcaatcaaagcctgtcattattgcaggtaatggaacaagtttaaaatcaattgactatagtttattacctaaagattatgatgttttccgttgcaatcaattttattttgaggatcattattttcttggtaagaaaataaaaaaggtattttttaattgttctgtaatttttgaacaatactatacgtttatgcaattaattaaaaataatgaatatgaatatgctgatattattctatcatcttttctgaatttaggggattcagaattaaagaaaatccagcgtttagaaaaattactaccacaaatcgatcttggtcatagctatttaaaaaaactacgagcttttgatgctcatttacaatatcacgaactatatgagaataagaggattacatcaggcgtttatatgtgtgcagtggcaactgctatgggttataaagatctttatttgacaggcattgatttttatcaagaaaaagggaatccttacgcatttcatcatcaaaaagaaaatattattaaattattaccttctttttcacaaaataaaagtcaaaatgatatccattctatggaatatgatttaaatgcactttatttcttacaaaaacattatggtgtaaatatttactgtatttcgccagaaagtcctctatgtaattattttcctttatcaccactgaataacccatttacttttattcccgaagaaaagaaaaattacacacaagatattttaattccgccagagtcagtgtataaaaaaattggtatatattccaaaccaagaatttaccaaaatctggtttttcggttgatctgggatatattacgtttacctaatgatataaaaaaagctttgaaagcaaagaaaatgagactacgcaaataa

SEQ ID NO: 18 - DNA, 1115 bp, Neisseria meningitidis
atgggcttgaaaaaggcttgtttgaccgtgttgtgtttgattgttttttgtttcgggatattttatacatttgaccgggtaaatcagggggaaaggaatgcggtttccctgctgaaggagaaacttttcaatgaagagggggaaccggtcaatctgattttctgttataccatattgcagatgaaggtggcggaaaggattatggcgcagcatccgggcgagcggttttatgtggtgctgatgtctgaaaacaggaatgaaaaatacgattattatttcaatcagataaaggataaggcggagcgggcgtactttttccacctgccctacggtttgaacaaatcgtttaatttcattccgacgatggcggagctgaaggtaaagtcgatgctgctgccgaaagtcaagcggatttatttggcaagtttggaaaaagtcagcattgccgcctttttgagcacttacccggatgcggaaatcaaaacctttgacgacgggacaggcaatttaattcaaagcagcagctatttgggcgatgagttttctgtaaacgggacgatcaagcggaattttgcccggatgatgatcggagattggagcatcgccaaaacccgcaatgcttccgacgagcattacacgatattcaagggtttgaaaaacattatggacgacggccgccgcaagatgacttacctgccgctgttcgatgcgtccgaactgaagacgggggacgaaacgggcggcacggtgcggatacttttgggttcgcccgacaaagagatgaaggaaatttcggaaaaggcggcaaaaaacttcaaaatacaatatgtcgcgccgcatccccgccaaacctacgggctttccggcgtaaccacattaaattcgccctatgtcatcgaagactatattttgcgcgagattaagaaaaacccgcatacgaggtatgaaatttatacctttttcagcggcgcggcgttgacgatgaaggattttcccaatgtgcacgtttacgcattgaaaccggcttcccttccggaagattattggctcaagccggtgtatgccctgtttacccaatccggcatcccgattttgacatttgacgataaaaatta

SEQ ID NO: 19 - DNA, 1230 bp, Escherichia coli
atgatatttgatgctagtttaaagaagttgaggaaattatttgtaaatccaattgggtttttccgtgactcatggttttttaattctaaaaataagactgaaaagctattgtcacctttaaaaataaaaaacaaaaatatttttattgttgttcatttagggcaattaaagaaagcagagctttttatacaaaaatttagtaagcgtagtaattttcttatcgtcttggcaactaaaaaaaacactgaaatgccaagattagttcttgagcaaatgaataaaaagttgttttcttcatataaactactatttataccaacagagccaaatacattttcgcttaaaaaagttatatggttttataatgtatataaatatatagttttaaattcaaaagctaaagatgcttattttatgagctatgcacaacattatgcaatcttcatatggttgttcaaaaaaaacaatataagatgttcattaattgaagaggggacagcgacgtataaaacagagaaaaaaaacacactagtaaatattaatttttattcgtggatcattaattcaattatcttgttccattatccagatttaaaatttgaaaatgtatacggcacctttccaaatttgttaaaagaaaaatttgatgcaaaaaaattttttgagtttaaaactattccattagttaaatcgtcaacaagaatggataatctcatacataaatatcgtatcactagagatgatattatatatgtaagtcaaagatattggattgacaacgaattgtatgcgcattcattaatatctaccttgatgagaatagataaatctgataacgcaagagtttttataaaacctcaccctaaagaaactaaaaaacatattaatgcaattcaaggtgcaataaataaagcaaagcgtcgtgatataattattattgtagaaaaagactttttaatagagtcaataataaaaaaatgcaaaataaaacacttgattggattaacatcatcttctttggtatacgcatctttagtttataaagagtgtaagacatattcaatagcacctattattataaaattgtgtaataatgaaaaatcccaaaaagggactaatacgctgcgtctccatttcgatattttaaagaattttgataatgttaaaatattatcggatgatatatcatctccctctttgcacgataaaaggattttcttgggggagtaa

SEQ ID NO: 20 - DNA, 1149 bp, Escherichia coli
atgactagaaaaaaagtgctttgttttgtctttcgttatgattctcattttttagctttgaaaaatatttttgagcagatagatgttgattcatatgatttatttttttgctgcttggataattctctacaagagtttttaaaaaaaaatttagatgaaaagatagttgtattctatcctgatgactttgtttattttttcacttttattaatattgagtttattttttgttcaacaggagggaaggaccttcatgaaattgttaatgctgtaagaacaaaaaatacaataattatatcttgttttccgggcattgtccttacttctcagatagaagcttttatttcaaaatctaatagtcactatttacttattaactcccctaaagacattaaaacgtataaaaaaatttgtaaaataataggggttccttttaatggaattctttttggtccaccatggattaaaaatgtcaatatcaatgcaaaaagtgagaattcttgtcttatcgttgatcaagttaatgagcccttgacgccaataaagaggatagaatatgcacgttttttgattagagtaattcagaaacatccgcatatgaattttatttttaaaactcgaaatccttttatatcaccagagtcaattgtttttgatattaaggaatacattgaacgcttcgatttgaaaaatataacatttagcgatgataatattgattctttaatttctaaagttgaatattgtattacaatatcttcttcggtcgcaatatattgtctggctaataaaattaaggtttatttaataaatggatttaatcatacctgcaatggacaatgttatttttcaagatctggacttattgttgattataataagtttaattttaaacacattccacgtattaaaaaaaaatggatggaggagaacctttattactctagggatattcaaaataagattttgaatgatattttaaaaatgccgccaaatgttaatgttagggcttttggaattaaaagatctacattaattatattatttttgatctttttgaatttttttttctcattaggatcaaaaaaaataaaaacattgaaaaaaatccataaagttttattaaggtataagaaagatgatatttga

SEQ ID NO: 21 - DNA, 1017 bp, Escherichia coli
atgaaaaatgttggttttattgttacaaaatcagaaattggtggtgcacaaacatgggtaaatgaaatatctaaccttattaaagaggaatgtaatatatttcttattacatctgaagaaggatggctcacacataaagatgtctttgccggagtttttgtcataccaggtattaaaaaatattttgacttccttacattgtttaaattgagaaaaattttaaaagaaaataacatttcaacgttaatagcaagttctgctaatgccggagtttatgccaggttagttcgattactagtcgactttaaatgtatttatgtttcgcatggatggtcttgtttatataatggtggtcgcctaaaatcaattttttgcattgttgaaaaatacctttctttattaactgatgttatatggtgtgtttccaaaagtgatgaaaaaaaggcaattgagaatattggtataaaagaaccaaagataatcacagtatcgaattcagtgcctcagatgccgagatgtaataataaacaactccagtataaggttctgtttgttggtaggttaacacaccctaagcgccccgaattgttagcgaatgtaatatcgaaaaagccccagtatagcctccatatcgtaggagggggggaaaggttagaatcattgaagaaacaattcagtgaatgtgaaaatattcattttttgggtgaggtcaataatttttataactatcatgagtatgatttattttcactgatatccgatagtgaaggtttgcctatgtcaggccttgaggctcacacagctgcaataccactcctgttaagtgatgtgggcggatgttttgaattaattgagggtaatgggttacttgtggaaaatactgaagacgacattggatataaattggataaaatattcgatgactatgaaaattatcgggaacaggcaattcgtgcctccgggaaatttgttatcgagaactatgcttcagcatataaaagcattattttaggttga

SEQ ID NO: 22 - DNA, 843 bp, Neisseria sp.
atgcaaaaccacgttatcagcttggcttccgccgcagagcgcagggcgcacattgccgataccttcggcagtcgcggcatcccgttccagtttttcgacgcactgatgccgtctgaaaggctggaacgggcgatggcggaactcgtccccggcttgtcggcgcacccttatttgagcggagtggaaaaagcctgctttatgagccacgccgtattgtgggaacaggcgttggacgaaggcttaccgtatatcgccgtatttgaagatgatgtcttactcggcgaaggcgcggagcagttccttgccgaagatacttggctgcaagaacgctttgaccccgattccgcctttgtcgtccgcttggaaacgatgtttatgcacgtcctgacctcgccctccggcgtggcggactacggcgggcgcgcctttccgcttttggaaagcgaacactgcgggacggcgggctatattatttcccgaaaggcgatgcgttttttcttggacaggtttgccgttttgccgcccgaacgcctgcaccctgtcgatttgatgatgttcggcaaccctgacgacagggaaggaatgccggtttgccagctcaatcccgccttgtgcgcccaagagctgcattatgccaagtttctcagtcaaaacagtatgttgggtagcgatttggaaaaagatagggaacaaggaagaagacaccgccgttcgttgaaggtgatgtttgacttgaagcgtgctttgggtaaattcggtagggaaaagaagaaaagaatggagcgtcaaaggcaggcggagcttgagaaagtttacggcaggcgggtcatattgttcaaatag

SEQ ID NO: 23 - DNA, 840 bp, Neisseria meningitidis
atgcagaaccacgtgatttccctggcttcagcggccgagcgccgtgctcatattgctgccacctttggtagtcgtggaatccctttccagttcttcgatgccctgatgccttcagaacgtctggagcaggcaatggcggagctggtccctggtctgtcagcccatccttatctgtctggcgttgaaaaagcgtgtttcatgtcccatgctgtcctgtgggaacaagccctggatgagggtctgccgtatatcgccgtgtttgaggacgatgtgctgctgggtgaaggtgctgaacagtttctggccgaggacacttggctggaagagcgtttcgataaagactcagcgttcattgtccgtctggagacaatgtttatgcacgtgctgacttctccatctggtgtagccgattatggcggtcgtgcctttcctctgctggagtccgaacactgtggtacagccgggtatattatcagccgtaaagccatgcgtttctttctggatcgttttgctgtgctgcctccggagcgcctgcatcctgttgatctgatgatgtttggcaatcctgatgaccgtgagggtatgccagtttgtcagctgaatccggcactgtgtgctcaggaactgcattatgccaaatttcacgaccagaatagcgctctgggaagtctgattgaacatgatcgtcgcctgaaccgtaaacaacagtggcgtgatagtccggctaacacgtttaaacaccgcctgattcgtgctctgaccaaaattggccgtgagcgtgaaaaacgtcgtaaacgccgtgaacagacgattgggaaaatcattgtgccattccagtga

SEQ ID NO: 24 - DNA, 996 bp, Escherichia coli
atgaacgataacgttttgctcataggagcttccggattcgtaggaacccgactacttgaaacggcaattgctgactttaatatcaagaacctggacaaacagcagagccacttttatccagaaatcacacagattggtgatgttcgtgatcaacaggcactcgaccaggcgttagccggttttgacactgttgtactactggcagcggaacaccgcgatgacgtcagccctacttctctctattatgatgtcaacgttcagggtacccgcaatgtgctggcggccatggaaaaaaatggcgttaaaaatatcatctttaccagttccgttgctgtttatggtttgaacaaacacaaccctgacgaaaaccatccacacgaccctttcaaccactacggcaaaagcaagtggcaggcggaggaagtgctgcgtgaatggtataacaaagcaccaacagaacgttcattaactatcatccgtcctaccgttatcttcggtgaacgcaaccgcggtaacgtctataacttgctgaaacagatcgctggcggcaagtttatgatggtgggcgcagggactaactataagtccatggcttatgttggaaacattgttgagtttatcaagtacaaactgaagaatgttgccgcaggttacgaggtttataactacgttgataagccagacctgaacatgaaccagttggttgctgaagttgaacaaagcctgaacaaaaagatcccttctatgcacttgccttacccactaggaatgctgggtggatattgctttgatatcctgagcaaaattacgggcaaaaaatacgctgtcagctctgtgcgcgtgaaaaaattctgcgcaacaacacagtttgacgcaacgaaagtgcattcttcaggttttgtggcaccgtatacgctgtcgcaaggtctggatcgaactctgcagtatgaattcgtccatgccaaaaaagacgacataacgtttgtttctgagtaa

SEQ ID NO: 25 - protein, 207 aa, Escherichia coli
MSKKLIIFGAGGFSKSIIDSLNHKHYELIGFIDKYKSGYHQSYPILGNDIADIENKDNYYYFIGIGKPSTRKHYLNIIRKHNLRLINIIDKTAILSPNIILGDGIFIGKMCILNRDTRIHDAVVINTRSLIEHGNEIGCCSNISTNVVLNGDVSVGEETFVGSCTVVNGQLKLGSKSIIGSGSVVIRNIPSNVVVAGTPTRLIRGNE

SEQ ID NO: 26 - protein, 346 aa, Escherichia coli
MSNIYIVAEIGCNHNGSVDIAREMILKAKEAGVNAVKFQTFKADKLISAIAPKAEYQIKNTGELESQLEMTKKLEMKYDDYLHLMEYAVSLNLDVFSTPFDEDSIDFLASLKQKIWKIPSGELLNLPYLEKIAKLPIPDKKIIISTGMATIDEIKQSVSIFINNKVPVGNITILHCNTEYPTPFEDVNLNAINDLKKHFPKNNIGFSDHSSGFYAAIAAVPYGITFIEKHFTLDKSMSGPDHLASIEPDELKHLCIGVRCVEKSLGSNSKVVTASERKNKIVARKSIIAKTEIKKGEVFSEKNITTKRPGNGISPMEWYNLLGKIAEQDFIPDELIIHSEFKNQGE

SEQ ID NO: 27 - protein, 418 aa, Escherichia coli
MRTKIIAIIPARSGSKGLRNKNALMLIDKPLLAYTIEAALQSEMFEKVIVTTDSEQYGAIAESYGADFLLRPEELATDKASSFEFIKHALSIYTDYESFALLQPTSPFRDSTHIIEAVKLYQTLEKYQCVVSVTRSNKPSQIIRPLDDYSTLSFFDLDYSKYNRNSIVEYHPNGAIFIANKQHYLHTKHFFGRYSLAYIMDKESSLDIDDRMDFELAITIQQKKNRQKILYQNIHNRINEKRNEFDSVSDITLIGHSLFDYWDVKKINDIEVNNLGIAGINSKEYYEYIIEKELIVNFGEFVFIFFGTNDIVVSDWKKEDTLWYLKKTCQYIKKKNAASKIYLLSVPPVFGRIDRDNRIINDLNSYLRENVDFAKFISLDHVLKDSYGNLNKMYTYDGLHFNSNGYTVLENEIAEIVK

SEQ ID NO: 28 - protein, 391 aa, Escherichia coli
MKKILYVTGSRAEYGIVRRLLTMLRETPEIQLDLAVTGMHCDNAYGNTIHIIEQDNFNIIKVVDININTTSHTHILHSMSVCLNSFGDFFSNNTYDAVMVLGDRYEIFSVAIAASMHNIPLIHIHGGEKTLANYDEFIRHSITKMSKLHLTSTEEYKKRVIQLGEKPGSVFNIGSLGAENALSLHLPNKQELELKYGSLLKRYFVVVFHPETLSTQSVNDQIDELLSAISFFKNTHDFIFIGSNADTGSDIIQRKVKYFCKEYKFRYLISIRSEDYLAMIKYSCGLIGNSSSGLIEVPSLKVATINIGDRQKGRVRGASVIDVPVEKNAIVRGINISQDEKFISVVQSSSNPYFKENALINAVRIIKDFIKSKNKDYKDFYDIPECTTSYD

SEQ ID NO: 29 - protein, 382 aa, Escherichia coli
MTRKKVLCFVFRYDSHFLALKNIFEQIDVDSYDLFFCCLDNSLQEFVKKNLDEKIVVFYPDDFVCFFTFINIEFIFCSTGGKDLHEIVNTVRTKDTIIISCFPGIVLTSQIEAFISKSNSHYLLINSPKDIKTYKKICKIIGVPFNGILFGPPWIKNVNINAKSENSCLIVDQVNEPLTPIKRIEYARFLIRVIQKHPHMNFIFKTRNPLISPDSIVFDIKEYIERFDLKNITFSDDNIDSLISKVEYCITISSSVAIYCLANKIKVYLINGFNHTCNGQCYFSRSGLIVDYNKFNFKHIPRIKKKWMEENFYYSRDIQHKILNDILKMPSNVNVRTFGIKRSTLIILFLIFFNFFFSLGPKKIKTLKKIHKVLLRYKKDDI

SEQ ID NO: 30 - protein, 409 aa, Escherichia coli
MIFDASLKKLRKLFVNPIGFFRDSWFFNSKNKAEELLSPLKIKSKNIFIISNLGQLKKAESFVQKFSKRSNYLIVLATEKNTEMPKIIVEQINNKLFSSYKVLFIPTFPNVFSLKKVIWFYNVYNYLVLNSKAKDAYFMSYAQHYAIFVYLFKKNNIRCSLIEEGTGTYKTEKENPVVNINFYSEIINSIILFHYPDLKFENVYGTYPILLKKKFNAQKFVEFKGAPSVKSSTRIDNVIHKYSITRDDIIYANQKYLIEHTLFADSLISILLRIDKPDNARIFIKPHPKEPKKNINAIQKAIKKAKCRDIILITEPDFLIEPVIKKAKIKHLIGLTSSSLVYAPLVSKRCQSYSIAPLMIKLCDNDKSQKGINTLRLHFDILKNFDNVKILSDDITSPSLHDKRIFLGE

SEQ ID NO: 31 - DNA, 1206 bp, Escherichia coli
atgcaaggtaatgcactaaccgttttattatccggtaaaaaatatctgctattgcaggggccgatgggaccttttttcaatgacgtcgccgaatggttagagtcattaggccgtaacgctgtgaatgttgtattcaacggtggggatcgtttttactgccgtcatcgacaatacctggcttactaccaaacgccgaaagagtttcccggatggttacgggatctccatcggcaatatgactttgataccatcctctgctttggtgactgccgcccattgcacaaagaagcaaaacgctgggcaaagtcgaaagggatccgctttctggcatttgaggaaggatatttacgcccgcaatttattaccgttgaagaagacggagtgaacgcatattcatcgctaccgcgcgatccggatttttatcgtaagttaccagatatgcctacgccgcacgttgagaacttaaaaccttcaacgatgaaacgtataggtcatgcgatgtggtattacctgatgggctggcattaccgccatgagttccctcgctaccgccaccataaatcgttttccccctggtatgaagcacgttgctgggttcgtgcatactggcgcaagcaactttacaaggtaacacagcgtaaggtattaccgaggttaatgaacgagttggaccagcgttattatcttgccgttttgcaggtatataacgatagccagattcgtaaccacagcaattataacgatgtgcgtgactatattaatgaagtcatgtactcattttcacgtaaagcgccgaaagaaagttatttggtgatcaaacatcatccgatggatcgtggtcacagactctatcgaccattaattaagcggttaagtaaggaatatggcttaagtgagcgcgtcatttatgtgcacgatctcccgatgccggaactattacgccacgcaaaagcggtggtgacgattaacagtacggcggggatctctgcactgattcataacaaaccactcaaagtgatgggcaatgccctgtacgacatcaagggcttgacgtatcaagggcatttgcaccagttctggcaggccgattttaaaccggatatgaaactgtttaagaagtttcgggggtatttattgatgaagacgcaggttaattgggtttattatggggggaacacaacaaactgccaacataatatatattaa

SEQ ID NO: 32 - DNA, 2028 bp, Escherichia coli
atgattggcatttactcgcctggcatctggcgtattccgcatctggagaaatttctggcgcaaccgtgccagaaactttctctgctgcgccctgttccgcaagaagttaatgctatcgccgtgtggggacatcgtcccagcgcggcgaaaccagtcgccatcgccaaagcagcgggaaaacccgtcattcgtctggaagatggatttgtgcgttcgctggatcttggcgtcaatggcgagccgccgctttctctggtggtggatgattgtggcatttactacgatgccagcaagccttcggcgctggagaaactggtacaggataaagccggaaatacagctctgataagccaggccagagaagcgatgcacaccatcgtgaccggggatatgtcgaaatataatctggcgcctgcgtttgtggctgatgagtcagaacgtacaaacatcgttctggttgtcgatcagacatttaatgatatgtcagtgacgtatggcaatgctggcccgcatgagtttgctgccatgctggaagccgcgatggcggaaaatcctcaagccgaaatttgggtgaaggtgcacccagatgtactggaaggaaagaaaacaggttatttcgccgatctgcgcgccacgcaacgagtacgtttaattgccgagaatgtcagcccgcagtcgctgttgcgacacgtttcccgggtttacgtcgtgacctcccaatacggctttgaagccttgctggcaggaaaaccagtaacatgtttcggccagccctggtatgcaagctggggcttaaccgacgatcgccatccgcagtccgctttgttatctgcccgacgcggttctgccacgctggaggaactttttgccgctgcatacctgcgttactgtcgctatatcgatccgcaaacgggagaagtaagcgatctatttaccgtgctgcaatggctgcaattacaacgtcgacatctgcaacagcgtaatggttatttatgggcgccaggcttaacgctgtggaagtcggcgatcctgaaacccttcttacgaacgccaacaaaccggctgagtttttcacgtcgctgtactgcggcgagcgcctgcgtggtatggggtgtaaagggggaacagcaatggcgagccgaagcgcagcgaaaatcactgccattatggcgaatggaagatggttttctgcgttcatccggacttggctctgacctgctgccgccgctatcgttggtactggataaacgcgggatctactatgacgccacgcgccccagcgacctggaagtgctgcttaatcatagccagctaacgctggcgcagcagatgcgagctgaaaaattacgccagcgactggttgaaagtaaactgagcaagtacaacctgggagccgatttctctctaccagccaaagccaaagataaaaaagttatcctggtgccgggtcaggtagaggacgatgcctctattaaaacaggcacagtctcgattaagagcaaccttgagttattacgcacagtacgcgagcgtaatccgcacgcctacattgtttataaaccgcacccggatgtactggtggggaatcgcaagggcgatattccggcagaactgactgctgaactcgctgattatcaggcactggacgccgatattattcaatgcattcagcgcgcagatgaagtgcataccatgacgtcgctgtcggggtttgaagcgttattacatggcaagcacgtacattgttacggcctgcccttctatgccggttggggtttaaccgtcgatgaacatcgttgcccgcgtcgcgagcgaaaattaacgttagcggatttgatctatcaggcgctgattgtttatccaacctatatccacccaacacggctacaacctattacggttgaagaggcggcggaatatttgatccagacaccgcgcaagccgatgtttattacccgaaaaaaagcggggcgagtaatacgttattaccgcaaattaattatgttctgtaaggtcagatttggctaa

SEQ ID NO: 33 - protein, 401 aa, Escherichia coli
MQGNALTVLLSGKKYLLLQGPMGPFFNDVAEWLESLGRNAVNVVFNGGDRFYCRHRQYLAYYQTPKEFPGWLRDLHRQYDFDTILCFGDCRPLHKEAKRWAKSKGIRFLAFEEGYLRPQFITVEEDGVNAYSSLPRDPDFYRKLPDMPTPHVENLKPSTMKRIGHAMWYYLMGWHYRHEFPRYRHHKSFSPWYEARCWVRAYWRKQLYKVTQRKVLPRLMNELDQRYYLAVLQVYNDSQIRNHSNYNDVRDYINEVMYSFSRKAPKESYLVIKHHPMDRGHRLYRPLIKRLSKEYGLSERVIYVHDLPMPELLRHAKAVVTINSTAGISALIHNKPLKVMGNALYDIKGLTYQGHLHQFWQADFKPDMKLFKKFRGYLLMKTQVNWVYYGGNTTNCQHNIY

SEQ ID NO: 34 - protein, 675 aa, Escherichia coli
MIGIYSPGIWRIPHLEKFLAQPCQKLSLLRPVPQEVNAIAVWGHRPSAAKPVAIAKAAGKPVIRLEDGFVRSLDLGVNGEPPLSLVVDDCGIYYDASKPSALEKLVQDKAGNTALISQAREAMHTIVTGDMSKYNLAPAFVADESERTNIVLVVDQTFNDMSVTYGNAGPHEFAAMLEAAMAENPQAEIWVKVHPDVLEGKKTGYFADLRATQRVRLIAENVSPQSLLRHVSRVYVVTSQYGFEALLAGKPVTCFGQPWYASWGLTDDRHPQSALLSARRGSATLEELFAAAYLRYCRYIDPQTGEVSDLFTVLQWLQLQRRHLQQRNGYLWAPGLTLWKSAILKPFLRTPTNRLSFSRRCTAASACVVWGVKGEQQWRAEAQRKSLPLWRMEDGFLRSSGLGSDLLPPLSLVLDKRGIYYDATRPSDLEVLLNHSQLTLAQQMRAEKLRQRLVESKLSKYNLGADFSLPAKAKDKKVILVPGQVEDDASIKTGTVSIKSNLELLRTVRERNPHAYIVYKPHPDVLVGNRKGDIPAELTAELADYQALDADIIQCIQRADEVHTMTSLSGFEALLHGKHVHCYGLPFYAGWGLTVDEHRCPRRERKLTLADLIYQALIVYPTYIHPTRLQPITVEEAAEYLIQTPRKPMFITRKKAGRVIRYYRKLIMFCKVRFG

SEQ ID NO: 35 - protein, 713 aa, Campylobacter jejuni
MLKKEYLKNPYLVLFAMIVLAYVFSVFCRFYWVWWASEFNEYFFNNQLMIISNDGYAFAEGARDMIAGFHQPNDLSYYGSSLSTLTYWLYKITPFSFESIILYMSTFLSSLVVIPIILLANEYKRPLMGFVAALLASVANSYYNRTMSGYYDTDMLVIVLPMFILFFMVRMILKKDFFSLIALPLFIGIYLWWYPSSYTLNVALIGLFLIYTLIFHRKEKIFYIAVILSSLTLSNIAWFYQSAIIVILFALFALEQKRLNFMIIGILGSATLIFLILSGGVDPILYQLKFYIFRSDESANLTQGFMYFNVNQTIQEVENVDFSEFMRRISGSEIVFLFSLFGFVWLLRKHKSMIMALPILVLGFLALKGGLRFTIYSVPVMALGFGFLLSEFKAILVKKYSQLTSNVCIVFATILTLAPVFIHIYNYKAPTVFSQNEASLLNQLKNIANREDYVVTWWDYGYPVRYYSDVKTLVDGGKHLGKDNFFPSFSLSKDEQAAANMARLSVEYTEKSFYAPQNDILKSDILQAMMKDYNQSNVDLFLASLSKPDFKIDTPKTRDIYLYMPARMSLIFSTVASFSFINLDTGVLDKPFTFSTAYPLDVKNGEIYLSNGVVLSDDFRSFKIGDNVVSVNSIVEINSIKQGEYKITPIDDKAQFYIFYLKDSAIPYAQFILMDKTMFNSAYVQMFFLGNYDKNLFDLVINSRDAKVFKLKI

SEQ ID NO: 36 - protein, 6 aa, Artificial Sequence
  Description of Artificial Sequence Synthetic 6xHis tag
HHHHHH

SEQ ID NO: 37 - protein, 5 aa, Artificial Sequence
  Description of Artificial Sequence Synthetic peptide
DQNAT